(19) United States
(12) Patent Application Publication
(10) Pub. No.: US 2023/0402183 A1
(43) Pub. Date: Dec. 14, 2023

(54) CARDIOVASCULAR DISEASE RISK ASSESSMENT SYSTEMS AND USES THEREOF

(71) Applicant: UNIVERSITY OF CONNECTICUT, FARMINGTON, CT (US)

(72) Inventors: Beiyan Zhou, Farmington, CT (US); Chuan Li, Farmington, CT (US)

(21) Appl. No.: 18/032,297

(22) PCT Filed: Oct. 18, 2021
(86) PCT No.: PCT/US2021/055455

§ 371 Date: Apr. 17, 2023

Related U.S. Application Data
(60) Provisional application No. 63/093,014, filed on Oct. 16, 2020.

(51) Int. Cl. G16H 50/30; G16H 50/20; C12Q 1/6883; G06N 20/00; G16H 50/70; G16B 25/00; G16B 30/00

(52) U.S. Cl. CPC G16H 50/30; G16H 50/20; C12Q 1/6883; G06N 20/00; G16H 50/70; G16B 25/00; G16B 30/00; C12Q 2600/158

(57) ABSTRACT

Disclosed herein are systems and methods for effectively predicting cardiovascular disease risk in people with normal lipid profiles and/or in people undergoing lipid management therapy. Genetic information obtained from a patient may be characterized. A logistic regression model may be applied to the characterized genetic information to estimate a probability of a cardiovascular event by the patient.

### CROSS-REFERENCE TO RELATED APPLICATIONS

This application claims priority to U.S. Provisional Application No. 63/093,014, filed Oct. 16, 2020, the entire contents of which are incorporated herein by reference.

### BACKGROUND

There are several existing cardiovascular disease (CVD) risk assessment systems in clinical practice, such as the JAMA Score (2001) and the Framingham risk score (2008). However, to date, there are no known systems that can effectively predict CVD risk in people with normal lipid profiles or in people undergoing lipid management therapy. In such patient populations, these individuals typically have optimal or near optimal lipid profiles, which have hindered appropriate risk assessment, and has made these patients unaware of potential CVD risks. Accordingly, a system that can effectively predict CVD risk in people with normal lipid profiles and/or in people undergoing lipid management therapy would offer an unmet medical need to reveal risk factors in populations for which effective predictions are not made.

### SUMMARY

In one aspect, disclosed herein are methods of predicting a diabetes and/or obesity risk of a patient, comprising: characterizing genetic information obtained from sequencing data of monocytes of the patient; and applying a logistic regression model to the characterized genetic information to estimate a probability of a cardiovascular event by the patient.

In some embodiments wherein characterizing cardiovascular risk of a patient, the genetic information is based upon an expression pattern associated with inflammatory macrophage activity.

In some embodiments wherein characterizing cardiovascular risk of a patient, characterizing the genetic information includes comparing the genetic information to a candidate gene set.

In some embodiments wherein characterizing cardiovascular risk of a patient, the candidate gene set comprises functionally interrelated genes, each gene of the candidate gene set being associated with at least one of the following functions: inflammation or immune response, lipid transport or metabolism, and cell proliferation or development.

In some embodiments wherein characterizing cardiovascular risk of a patient, applying the logistic regression model further includes application to physiological feature data and demographic data associated with the subject.

In some embodiments wherein characterizing cardiovascular risk of a patient, applying the logistic regression model includes input of the generated MPI score to the model to estimate the probability of the cardiovascular event.

In some embodiments wherein characterizing cardiovascular risk of a patient, characterizing the genetic information includes generating a macrophage-derived foam cell index (MDFI) score, and wherein applying the logistic regression model includes input of the generated MDFI score to the model to estimate the probability of the cardiovascular event.

In some embodiments wherein characterizing cardiovascular risk of a patient, characterizing the genetic information further includes generating a macrophage polarization index (MPI) score.

In some embodiments wherein characterizing cardiovascular risk of a patient, the method further comprises displaying an output of the estimated probability.

In another aspect, disclosed herein, are computer systems for predicting a cardiovascular risk of a patient, comprising: a data source providing sequencing data of monocytes obtained from the patient; and a processor communicatively coupled to the data source, wherein the processor is configured to: characterize genetic information obtained from the sequencing data based upon an expression pattern associated with inflammatory foaming macrophage activity; and apply a logistic regression model to the characterized genetic information to estimate a probability of a cardiovascular event by the patient.

In some embodiments of the computer system for predicting a cardiovascular risk, the logistic regression model includes model coefficients determined based on expression patterns and outcome reports of patients for which cardiovascular events previously occurred.

In another aspect, disclosed where are methods of predicting diabetes and/or obesity risk of a patient, comprising: characterizing genetic information obtained from sequencing data of monocytes of the patient; and applying a logistic regression model to the characterized genetic information to estimate a probability of a cardiovascular event by the patient.

In some embodiments of the methods of predicting diabetes and/or obesity risk, characterizing the genetic information is based upon an expression pattern associated with inflammatory macrophage activity.

In some embodiments of the methods of predicting diabetes and/or obesity risk, characterizing the genetic information includes comparing the genetic information to a candidate gene set.

In some embodiments of the methods of predicting diabetes and/or obesity risk, the candidate gene set comprises functionally interrelated genes, each gene of the candidate gene set being associated with at least one of the following functions: inflammation or immune response, lipid transport or metabolism, and cell proliferation or development.

In some embodiments of the methods of predicting diabetes and/or obesity risk, the method comprises applying the logistic regression model further includes application to physiological feature data and demographic data associated with the subject.

In some embodiments of the methods of predicting diabetes and/or obesity risk, characterizing the genetic information further includes generating a macrophage polarization index (MPI) score.

In some embodiments of the methods of predicting diabetes and/or obesity risk, further comprising displaying an output of the estimated probability.

In another aspect, disclosed herein are computer systems for predicting a diabetes and/or obesity risk of a patient, comprising: a data source providing sequencing data of monocytes obtained from the patient; and a processor communicatively coupled to the data source, wherein the processor is configured to: characterize genetic information obtained from the sequencing data based upon an expression pattern associated with inflammatory foaming macrophage activity.

### BRIEF DESCRIPTION OF THE FIGURES

FIG. 1 shows a schematic overview of the study. Substantial CVD risk remains in subjects of optimal/nearoptimal lipid status, referred as residual CVD risk. AtheroSpectrum revealed the pathogenic foaming program of atherogenic macrophages. By cross-referencing these genes with genes from peripheral monocytes from MESA participants with low LDL levels using our novel algorithm, 29 residual CVD risk signature genes were identified. By combining these 29 genes with other factors, we created a Residual Cardiovascular Risk score by 29 genes (RCR29) score that effectively depicted residual CVD risk.

FIGS. 2A-2C show substantial residual CVD risks were not explained by traditional risk factors in the MESA cohort. FIG. 2A shows the number of MESA participants taking any lipid-lowering medication in persons with ≤129 or >129 mg/dL LDL. FIG. 2B shows percentages of female or male subjects with CVEs with LDL>129 mg/dL or ≤129 mg/dL. FIG. 2C shows standard box-plots showing male vs female comparisons of HDL, LDL, fasting glucose, waist circumference, body-mass index (BMI), triglycerides, body fat, age, seated diastolic blood pressure (SDBP), seated systolic blood pressure (SSBP), and smoking status for subjects with LDL≤129 mg/dL that had CVEs or not OR=odds ratio.

FIGS. 3A-3G show AtheroSpectrum identified inflammatory/foaming macrophage populations associated with severity of atherosclerosis. FIG. 3A is a scheme showing the transition of different types of atherogenic macrophages by AtheroSpectrum. FIGS. 3B and 3C show AtheroSpectrum depicted 4 major sub-populations of murine plaque macrophages with different foaming and inflammatory properties and marker gene expression. FIG. 3D shows AtheroSpectrum identified different artery macrophage sub-population distributions in atherogenic vs. control mice, atherogenic vs. regression mice, and symptomatic vs. asymptomatic humans with atherosclerosis. FIG. 3E shows heatmaps showing signaling pathways and cell functions enriched in each of the artery macrophage sub-populations. FIG. 3F shows representative pathways fall into three main categories: inflammatory response, lipid transport/metabolism, and cell development and survival. FIG. 3G is a scheme showing predicted cell differentiation pathways towards homeostatic foaming and pathogenic (inflammatory) foaming phenotypes.

FIGS. 4A-4D show the present algorithm identified CVD signature genes that were significantly correlated with CVEs in the MESA subjects with LDL<129 mg/dL. FIG. 4A shows functional categories of the 29 signature genes. FIG. 4B shows the weights of the 29 signature genes and age, gender, and MPI in the regression analysis. FIG. 4C shows ATP8B4, RCC2, R3HCC1, and MRPL1 are associated with CVEs and had significantly different expression levels in athero vs. non-athero artery tissues of human subjects deposited at GTExPortal.org (211 non-athero, 697 athero). FIG. 4D shows centered expression values of ATP8B4, RCC2, R3HCC1, and MRPL1 in paired atheroma plaque vs. intact tissue from 32 patients (GSE43292). P values in FIGS. 4C and 4D were calculated by t-test with false discovery rate adjustment. FDR=false discovery rate.

FIGS. 5A-5C show RCR29 score effectively depicts residual CVD risks. FIG. 5A shows RCR29 scores, JAMA 2001 10-year (10 y) risk score, and the Framingham risk score for the MESA subjects with LDL≤129 mg/dL. FIG. 5B shows the accuracy of CVE prediction by RCR29 score in MESA subjects. FIG. 5C shows ROC plots and AUC values of RCR29 scores, JAMA 2001 10 y risk scores, and Framingham risk scores for the MESA subjects. Differently colored curves indicated individual runs of the 5-fold cross validation, and the average AUC of the 5 runs were shown.

FIG. 6 shows traditional CVD risk factors in MESA subjects with LDL>129 mg/dL. HDL, LDL, triglycerides, fasting glucose, waist circumstance, BMI, body fat, age, and smoking status of MESA subjects that had LDL>129 mg/dL and with or without CVEs.

FIG. 7 shows MPI-AMDI and AMDI-MFDI plots of murine athero-plaque macrophages, atherogenic vs. control (mice), atherogenic vs. regression (mice), and symptomatic vs. asymptomatic (human) atherosclerosis conditions.

FIGS. 8A-8C show AtheroSpectrun identified “inflammation/foaming” signature genes that are significantly altered in patients' arteries. FIG. 8A shows AtheroSpectrum identified 2209 “inflammation/foaming” signature genes that are specifically enriched in the inflammatory foaming macrophage subpopulation. FIG. 8B shows the top 10 pathways enriched in the 2209 “inflammatory foaming” signature genes. A major portion of the 2209 genes are significantly changed. FIG. 8C shows Genes in athero vs. non-athero artery tissues of human subjects deposited at GTExPortal.org.

FIG. 9 shows ATP8B4, RCC2, R3HCC1, and MRPL1, which are significantly associated with CVEs, had significantly different expression levels in athero vs. non-athero artery tissues of both female and male subjects deposited at GTExPortal.org. (female: 79 non-athero, 207 athero, male:132 non-athero, 490 athero).

FIGS. 10A-10D show RCR29 score effectively depicted residual CVD risks in females and males. FIG. 10A shows relative expression of the 29 residual risk signature genes in circulating monocytes of female or male MESA subjects with LDL≤129 mg/dL and who had a CVE (Yes) or not (No). FIG. 10B shows RCR29 scores, JAMA 2001 10 y risk scores, and Framingham risk scores in MESA subjects with LDL≤129 mg/dL who had a CVE or not. FIG. 10C shows the accuracy of CVE prediction by RCR29 score in MESA subjects. FIG. 10D shows ROC plots and AUC values of RCR29 scores, JAMA 2001 10 y risk scores, and Framingham risk scores in MESA subjects. Differently colored curves indicated individual runs of the 5-fold cross validation, and the average AUC of the 5 runs were shown.

FIG. 11 shows CVE-free days survived by MESA subjects. CVE-free days survived since monocyte collection of MESA subjects who were predicted to have CVE or not by RCR29 score. Females and males are plotted separately and together. Significance of RCR29 score in predicting different survival longevity was calculated with Cox regression.

FIG. 12A-12D show scRNA-seq profiles of cultured M0, M1, and M2 BMDMs and ATMs from lean and obese mice. FIG. 12A shows scRNA-seq scheme. Cultured M0, M1, and M2 BMDMs were barcoded separately and processed together for scRNA-seq. ATMs from lean and obese mice were barcoded separately and processed together for scRNA-seq. FIGS. 12B and 12C show t-SNE clustering of scRNA-seq from 6979 M0, 4736 M1, and 6391 M2 BMDMs (FIG. 12B), and combined data from BMDMs (M0, M1, and M2) and 1710 lean and 1758 obese ATMs (FIG. 12C). FIG. 12D shows expression of macrophage lineage markers (left panel), and well-known M1 (middle panel) and M2 markers (right panel) by t-SNE plots.

FIGS. 13A-13H show generation of the macrophage polarization index (MPI). FIG. 13A shows rₘ₁ versus rₘ₂ contour plots of 4736 M1 and M2 BMDMs using the top 500 most differentially expressed (absolute fold change) and significantly changed genes (FDR-adjusted P<1×10⁻¹⁰) (PSG). FIG. 13B shows polarization axis is the regression line of transcriptomes to M1 (rₘ₁) or M2 gene sets (rₘ₂). FIG. 13C shows MPI is calculated as l=P−P₀. FIG. 13D show macrophage distributions along the MPI scale. FIG. 13E shows PSG-enriched pathways. FIG. 13F shows a heatmap of PSG. Known markers for M1 or M2 are indicated. FIGS. 13G and 13H show MPI density distributions of human PBMC-derived macrophages stimulated with IFN-γ (GSE82227) (FIG. 13G) and murine BMDMs after Salmonella exposure (GSE65528) (FIG. 13H).

FIG. 14A-14B show a comparison of BMDM scRNA-seq profiles. FIG. 14A shows a similarity analyses of individual cells in M0, M1, and M2 BMDM samples were calculated using whole transcriptomes. An equal number (4736) of cells were randomly selected from each population; rows and columns represent individual cells from the 2 populations being compared, and the color of their crossing point represents adjusted correlation coefficient r; higher (yellow) and lower (blue) r suggest higher and lower similarity, respectively. FIG. 14B show bulk similarities between the M0, M1, and M2 BMDM populations as in FIG. 14A. Distances between populations are indicated next to the connecting lines; longer distances indicate more different from each other.

FIG. 15A-15E show generation of the activation-induced macrophage differentiation index (AMDI). FIG. 15A show the pathways significantly enriched in the 435 AMDSGs. FIG. 15B show macrophage distributions along the AMDI scale. FIGS. 15C and 15D show single-cell trajectory of M0, M1, and M2 BMDMs (4736 cells/sample) with Monocle using PSGs and AMDSGs colored by BMDM population (FIG. 15C) or pseudotime (FIG. 15D). FIG. 15E shows a heatmap showing smoothened relative expression of PSGs and AMDSGs along pseudotime progression, from M0 to M1 and M2 branches of the single-cell trajectory. Known signature genes are indicated. Hierarchical clustering generated 6 expression groups: groups I and III enriched in M1, group II enriched in M2 or M1 and M2, groups V and VI enriched in M0, group IV enriched in M0 or M2.

FIGS. 16A-16D show MacSpectrum characterization of visceral ATM subsets from lean and obese mice. FIG. 16A shows macrophage subsets on the MacSpectrum plot were designated as A, “M2-like”; B, “M1-like”; C, “transitional M1-like”; and D, “preactivation.” FIG. 16B shows MacSpectrum plot of 1710 lean and 1758 obese ATMs with percentages calculated for each region (A, B, C, or D). FIG. 16C shows pathways enriched in lean (L) and obese (O) region-specific (A, B, C, or D) subpopulations. FIG. 16D shows a heatmap of ATM signature genes identified using MacSpectrum. Each gene was plotted as one row along MPI; all genes arranged by hierarchical clustering.

FIGS. 17A-17E show MacSpectrum identified unique gene sets associated with diabetes conditions in obesity. FIG. 17A shows a heatmap showing expression of the 23 gene candidates identified using MacSpectrum in blood monocytes of 18 obese patients before and after bariatric surgery. FIGS. 17B and 17C show correlation coefficients (r) of the 23 gene candidates with patients' BMI (FIG. 17B) and HOMA-IR (FIG. 17C). FIGS. 17D and 17E show correlation (FIG. 17D) of PRKAG2, ERCC1, AMOTL2, and BDH2 with HOMA-IR and BMI and their microarray-determined relative expression (FIG. 17E) in visceral adipose tissue CD14⁺ cells from 12 obese patients with (Dia) or without diabetes (No Dia) (GSE54350). Data represent mean±SEM. *P<0.05, **P<0.01 by 2-tailed Welch's t test.

FIG. 18 shows a scheme showing the data processing pipeline of MacSpectrum.

FIG. 19. scRNA-seq of BMDMs and ATMs. FIG. 19A shows a Venn Diagram (non-proportional) of genes expressed in >1% of M0, M1, or M2 BMDMs and lean or obese ATMs. FIG. 19B shows t-SNE clustering of scRNA-seq from 1710 lean and 1758 obese ATMs. FIG. 19C show expression of classic M1 and M2 markers in ATMs by t-SNE plots. FIG. 19D shows t-SNE dimensional reduction of ATMs by cluster (left) and percentage (right); color bars representing log 2 transformed expression levels.

FIGS. 20A-20B show identification of gene sets allows efficient separation of BMDM M1 and M2 activation states. FIG. 20A shows rm1 vs. rm2 dot plot of M1 and M2 BMDMs including 11,315 genes expressed in >1% of M1 and/or M2 BMDMs. FIG. 20B shows rm1 vs. rm2 contour plot of M1 and M2 BMDMs including 11,315 genes expressed in >1% of M1 and/or M2 BMDMs. (C-E) rm1 vs. rm2 contour plots of M1 and M2 BMDMs using the top 3000, 1000, and 100 most differentially expressed (absolute fold change) and significantly changed genes (FDR-adjusted p<10-10).

FIGS. 21A-21I show Application of MPI to characterize expression patterns of signature genes from IFNγ-stimulated (GSE82227) and Salmonella-stimulated (GSE65528) macrophages. FIGS. 21A-21D show gene-specific expression by MPI plots of human PBMC-derived macrophages stimulated with IFNγ for 2, 6, and 24 hours; M1 (FIG. 21A) and M2 (FIG. 21C) activation genes reported in the original publication and additional M1 (FIG. 21B) and M2 (FIG. 21D) signature genes identified by MPI application; expression levels are log 2(DESeq-normalized counts+1). FIGS. 21E-21H show gene-specific expression by MPI plots of immortalized murine BMDMs 0, 2.5, 4, and 8 hours after Salmonella exposure; M1 (FIG. 21E) and M2 (FIG. 21G) activation genes reported in the original publication and additional M1 (FIG. 21F) and M2 (FIG. 21H) signature genes identified in the present study; expression levels are log 2(TPM+1); Blue curves are loess smoothing lines. FIG. 21I shows rm1 vs. rm2 dot plot of the entire dataset.

FIGS. 22A-22F show successful separation of BMDMs with various activation states using PSGs and AMDSGs. FIG. 22A shows macrophage distributions along the MPI scale. FIG. 22B shows t-SNE analyses of BMDM M0/M1/M2 and ATM transcriptomes with PSGs and AMDSGs. Single-cell transcriptome profiles of M0, M1, M2 BMDMs (4736 cells from each sample) were analyzed using cell trajectory algorithms with the whole transcriptome (FIG. 22C) that displayed overlapping of M0 and M2 populations. FIG. 22D shows M0, M1, and M2 BMDM separation by MacSpectrum. FIG. 22E shows a 3D plot of rm1, rm2, and rm0 (see Methods for calculation details) values from single-cell transcriptome profiles of M0, M1, and M2 populations. FIG. 22F shows murine aortic macrophages during atherosclerosis progression (baseline) and regression (GSE97941) depicted by MacSpectrum, with the four regions (A, B, C, and D) labelled.

FIGS. 23A-23D show MacSpectrum analysis of ATMs in obesity. FIGS. 23A-23D show expression of known classic M1 (FIG. 23A) and M2 genes (FIG. 23C), and additional M1 and M2 genes identified in this study (FIG. 23B, FIG. 23D) by MacSpectrum plot of total, lean, and obese ATMs. Expression levels are log 2(UMI counts+1). (E) t-SNE dimensional reduction of ATMs using PSGs and AMDSGs.

FIGS. 24A-25C show signaling pathway and regulator analysis of ATM subsets identified by the MacSpectrum. FIGS. 24A and 24B show enriched pathways (FIG. 24A) and upstream regulators (FIG. 24B) in region B vs. A ATMs identified by IPA; positive and negative z-scores indicate estimated activation or inhibition, respectively; all pathways and regulators displayed are significantly different at p<0.05. FIG. 24C show the most enriched biological functions in lean and obese ATMs in regions A and B compared to total (lean+obese) cells in region D identified by IPA; functions listed were significantly enriched (p<10-30) in at least one of the four comparisons (columns); positive and negative z-scores indicate estimated activation or inhibition, respectively.

FIGS. 25A-25C show MacSpectrum consistently identified reported features of murine ATMs. FIGS. 25A and 25B show enrichment of reported Cd9+ ATM signature genes in region C of obese ATMs. FIG. 25C show enrichment of dendritic cell signature genes in region B of obese ATMs. p-values in FIGS. 25A and 25C were determined by 2-tailed Welch's t-test, color bars in FIG. 25B represented log 2(UMI counts+1) of the genes.

FIGS. 26A-26E show MacSpectrum revealed dynamic changes of ATM subsets in obesity. Obese or lean ATMs in regions A and B were analyzed using NIH-DAVID Bioinformatics Resources for gene function (FIG. 26A) and subcellular location (FIG. 26B). FIG. 26C shows enrichment of transcription factor Spi1 (PU.1) in region C, and its targets Id2, Psmb9, and P2ry10 in region B of the obese ATMs. FIG. 26C shows enrichment of transcription factor Zeb2 in region C, and its targets Ccnd1 and Id2 in region B of the obese ATMs. FIG. 26E shows a schematic of ATM subpopulation changes during development of obesity. Under lean condition, a small quantity of less mature/preactivation (region D) reside in visceral adipose tissue; a dominant macrophage subset displays an “M2-like” phenotype (region A). Under an obese condition, recruitment of peripheral monocytes (region D) into adipose tissue is enhanced, which could replenish a tissue-maintenance “M2-like” subset (region A); more importantly, a large portion of them gave rise to an immature but highly inflammatory subpopulation (C) as an initial response to the pro-inflammatory micro-environment in the tissue. Continuous exposure to the pro-inflammatory cues further developed the region C cells into a subpopulation (B) bearing a mature and tuned pro-inflammatory profile; and those cells accounted for the later/more-persistent responses to obesity stress. Targets of regulators in FIGS. 26C and 26D were identified using IPA; expression levels in FIGS. 26C and 26D are log 2(UMI counts+1).

FIGS. 27A-27D show profiles of disease-associated gene sets identified by MacSpectrum. FIG. 27A shows Log2 fold changes and log 10p-values of the 603 genes that showed significant differential expression in obese vs lean monocytelike cells. The 385 genes that consistently displayed polarization-associated expression are colored red, p-values were determined by 2-tailed Welch's t-test. FIG. 27B shows pathways significantly enriched in the 436 genes that were expressed with distinct macrophage polarization pattern in murine ATMs and displayed significant differential expression (p<0.05) in CD14+ cells isolated from obese human subjects with or without diabetes (GSE54350). FIG. 27C shows pathways significantly enriched in the 23 gene candidates identified using MacSpectrum in blood monocytes of obese patients. FIG. 27D shows a Venn Diagram of the top 50% most abundant genes (average expressions) in obese human (GSE54350) and murine ATMs.

FIG. 28 shows a computer system for predicting risk of a patient in accordance with one or more implementations of the present disclosure.

FIG. 29 shows a method for predicting risk of a patient in accordance with one or more implementations of the present disclosure.

FIG. 30 shows a method for training a machine learning algorithm in accordance with one or more implementations of the present disclosure.

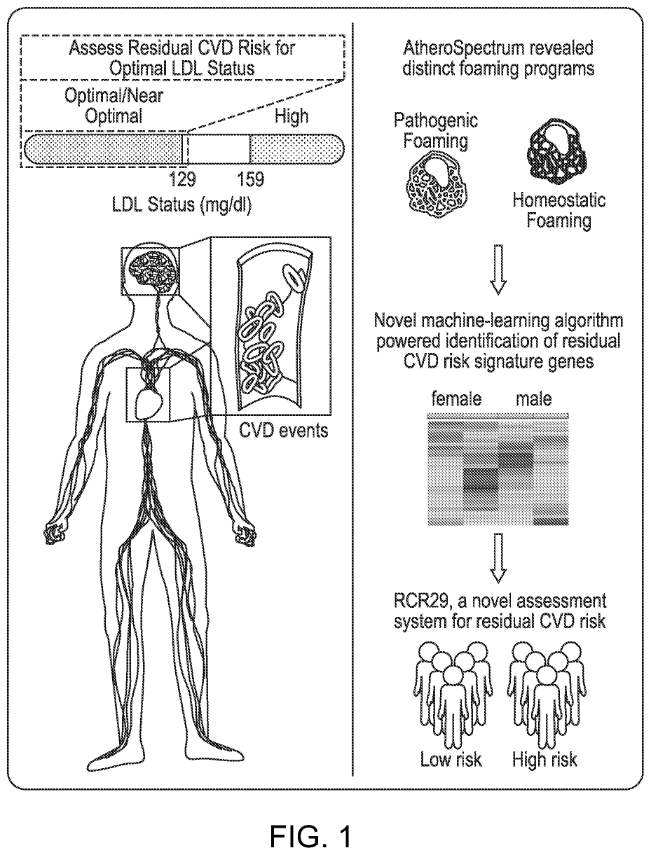

### DETAILED DESCRIPTION

Atherosclerosis remains one of the main causes of death worldwide even as several interventions have been shown to manage risk of atherosclerosis and related cardiovascular diseases (CVD). Tragically, residual risk of CVD has emerged as a relevant threat in populations with successful lipid and/or hypertension management and interventions to reduce known risk factors (fasting glucose, smoking, etc.). Indeed, several recent clinical trials revealed that substantial cardiovascular events (CVE) occur even in populations that have achieved optimal or near-optimal blood lipid profiles, and that these directly contribute to the leading causes of death worldwide.

Although recent analyses suggest other factors such as systemic inflammation and suboptimal lipid levels may contribute to CVD incidence, current therapeutic strategies remain ineffective in addressing these challenges. This is most likely due to an insufficient understanding of pathogenic mechanisms. To this end, a crucial task is to assess residual CVD risk in populations with well-managed known risk factors. Current tools for assessing CVD risk rely on traditional risk factors such as low-density lipoprotein (LDL), fasting glucose, smoking, etc., which have shown limited if any association with residual risk, making them ineffective predictive factors. The lack of efficient risk assessment approaches has made patients unaware of potential CVD risks and hindered appropriate interventions, which emphasizes the need for novel risk assessment tools. These tools should be initially aimed at factors already thought to be involved in CVD.

Monocytes and macrophages may represent critical factors for atherosclerosis as they both influence the development and progression of CVD and macrophage-derived foam cells are the most abundant cell type in atherosclerosis. Peripheral circulatory monocytes can sense metabolic and inflammatory cues, such as during plaque formation, and adjust their transcriptional programs accordingly. These responses significantly influence their behaviors after recruitment to atherogenic sites, which include maturation into tissue-residing macrophages, manipulation of inflammation and tissue remodeling, and/or further transformation into lipid-laden foam cells—where they orchestrate progression of atherosclerosis.

Given the central role of monocytes/macrophages in atherogenesis, extensive efforts have been invested in understanding their actions during recruitment and after infiltration into atherogenic foci, especially their lipid uptake/efflux relevant to this unique environment10-16. Currently, plaque-resident macrophages have been defined as pro-inflammatory “M1”-like macrophages that release proatherogenic cytokines and accelerate atherosclerosis progression, and alternatively activated “M2”-like macrophages believed to reduce plaque inflammation and suppress atherosclerosis progression. As a major component of atherosclerotic plaque, foam cells not only accumulate as fatty streaks, but also release pro-inflammatory cytokines and orchestrate pathological tissue remodeling. Indeed, specific macrophage/foam cell phenotypes can impact stability of atherosclerotic plaques differently. However, many studies fall short in explaining key features that can distinguish between symptomatic and asymptomatic outcomes in patients or depicting a comprehensive landscape that captures the intertwined inflammation and lipid handling aspects that are unique to macrophage-derived foam cells.

Adding to this complexity, monocytes carry critical and comprehensive information that influences their responses to specific physiological conditions of individual people. Such primed conditions can orchestrate individualized monocyte programs to guide their swift, fine-tuned, and diverse responses during atherogenesis and its progression with respect to cytokine production varieties and quantities, lipid handling efficiency, as well as cell-cell interaction in the atherogenic foci. However, such crucial information in circulating monocytes is difficult to extract from transcriptomics of bulk RNA-seq data, primarily due to relatively subtle gene expression patterns and lack of available high-resolution tools to characterize their dynamic and plastic actions. Associations between either monocyte counts or transcriptome profiles and CVD incidence are only found to be moderate with limited if any predictive capability, and these have never been investigated in the context of residual CVD risk.

Herein, a macrophage annotation program, AtheroSpectrum, was created, which revealed a pathogenic inflammatory foam cell-specific genetic program contributing to residual CVD risk. By combining this gene set with known CVD risk factors, a more accurate prediction of residual CVD risk was than previous models.

### Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the invention.

The terms “a,” “an,” and “the” and similar referents used in the context of describing the invention (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

As used herein, the term “about” refers to a range of values of plus or minus 10% of a specified value. For example, the phrase “about 200” includes plus or minus 10% of 200, or from 180 to 220, unless clearly contradicted by context.

As used herein, “optional” or “optionally” means that the subsequently described event or circumstance may or may not occur, and that the description includes instances where said event or circumstance occurs and instances where it does not.

CVD Risk

Herein, a macrophage annotation program, AtheroSpectrum, was created, which revealed a pathogenic inflammatory foam cell-specific genetic program contributing to residual CVD risk. By combining this gene set with known CVD risk factors, a more accurate prediction of residual CVD risk was than previous models.

The computer-implemented methods and systems described herein can include candidate gene lists comprising genes associated with inflammatory foaming macrophage activity. An example candidate gene list is provided in Table A herein.

TABLE A
Listing of 29 Genes for RCR 29

EIF3G
ATP8B4
CYLD
BIRC2
BIP2B
CCS
ALDH6A1
DYNLL1
RCC2
SLC39A8
RPS19
ASGR2
PRELID1
EPDR1
AGPAT4
CD302
SOS2
R3HCC1
EPB41L3
CANX
INTS10
UGT1A6
SOD2
RHOF
MRPL1
AOAH
CCDC71
RGS12

Obesity and Diabetes Risk

Adipose tissue inflammation and systemic insulin resistance are the hallmarks of obesity and causal factors of obesity-induced metabolic disorders such as prediabetic conditions and type 2 diabetes mellitus (T2DM). Adipose tissue macrophages (ATMs) are the most abundant immune population in obese visceral adipose tissue and play a central role in modulating adipose tissue function and homeostasis. The plasticity of macrophages allows swift, fine-tuned, and diverse responses to execute crucial functions during pathogen infection and tissue homeostasis. Such plasticity coupled with dynamic tissue- and stress-specific responses generates highly heterogeneous macrophage subpopulations with phenotypic complexity that is difficult to characterize. This challenge is exemplified by the confusing and conflicting results obtained when macrophages are characterized as single populations from specific tissues or in response to pathogenic challenges. The inability to define specific functional macrophage populations may underlie failures in the development of effective disease therapies; therefore, new approaches are needed.

Several models proposed to understand macrophage function define cells by either pathogen/antigen-specific responses or tissue-specific outcomes. The most popular macrophage polarization model separates macrophages into classical (M1) and alternative (M2) activation states, based on responses elicited by T helper 1 (Th1) (or other pathogens) and Th2 cell-derived cytokines, respectively. Other models separate M1 and M2 into subtypes based on limited features examined in different contexts. However, no currently available model allows comprehensive annotation of complex macrophage features under different conditions. Advances in high-throughput sequencing technologies allow in-depth analyses of cell populations to identify distinct subsets and dissect regulatory mechanisms underlying cell function. However, currently available algorithms that perform comparisons of transcriptomes from whole populations are unable to define complex and subtle differences among subpopulations within one lineage and have even less capacity to characterize the dynamic activation-state changes of each subpopulation. Hence, the results are often ambiguous when macrophages are analyzed as bulk samples using these methods, which weakens our understanding of human macrophages.

To identify critical signaling networks governing ATM action under healthy or obese conditions, single-cell transcriptome data to develop new algorithms for high-resolution macrophage analysis were generated. This resulted in the creation of a macrophage annotation platform) that can differentiate macrophage subpopulations by polarized activation state and terminal maturation stage. Two newly developed algorithms enable full-spectrum coverage and high-resolution annotation of macrophage subpopulations in vivo through the fine-mapping of each cell to a well-supported inflammatory or “terminal maturation” state. The algorithm divided ATMs into M1-like, M2-like, transitional, and preactivation phenotypes to allow further characterization. More importantly, MacSpectrum revealed unprecedented information about macrophage responses under obese conditions, including regulatory factors, signaling pathways, and diabetes-specific signature genes.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

### EXEMPLIFICATION

### Example 1: AtheroSpecrum Repeals Novel Macrophage Foam Cell Gene Signatures Associated with Residual CVD Risk

Summary

While several interventions can achieve effective lipid management in cardiovascular disease (CVD) patients, residual risks remain. This suggests an unmet medical need to reveal risk factors in populations with optimal/near optimal LDL levels. Monocytes and macrophages play central roles in atherosclerosis development, but previous work has yet to provide a detailed view of macrophage populations involved in CVD risk.

Using single-cell transcriptomics, we created a novel computational algorithm, AtheroSpectrum, with two quantitative indices depicting lipid metabolism and inflammatory status of macrophages in the atherogenesis loci. AtheroSpectrum revealed distinct macrophage-foaming programs and extracted 2209 differentially expressed genes associated with atherosclerosis. We then created a computing program to analyze their expression patterns in peripheral monocyte transcriptomics from 936 participants in the Multi-Ethnic Study of Atherosclerosis (MESA) with optimal/near-optimal low-density lipoprotein (LDL) levels (≤129 mg/dL). A list of 29 genes were identified and then incorporated with additional known risk factors that displayed significant correlation with CVD risk in the MESA cohort to create a CVD residual risk prediction model RCR29 that were further validated in two independent datasets (GTEx and GSE43292).

Using from three sets of single-cell transcriptomics profiles (GSE97310, GSE116240, SE97310), AtheroSpectrum detected two distinct programs in murine plaque macrophages: homeostatic-foaming and inflammatory pathogenic-foaming. The latter were positively associated with severity of atherosclerosis in multiple studies in human. Using a large pool of participant data from MESA and our original machine-learning algorithm, 29 signature genes strongly associated with cardiovascular events were identified in MESA, which were further validated in human artery datasets from two independent studies in which traditional risk factors showed no significant association. A novel residual CVD risk score model was then developed that incorporates the 29 signature genes with parameters that displayed sensitivity to CVD incidence in MESA, including sex, age, and the macrophage polarization index (MPI) produced by AtheroSpectrum. This score presented stronger predictive power of residual CVD risk (AUC 0.805) compared to the JAMA 2001 (AUC 0.620) and Framingham 2008 risk scores (AUC 0.650) in these datasets.

In sum, careful dissection of heterogeneous cell populations and underlying genetic programs can reveal hidden CVD risk factors. In this example, specific sub-populations of plaque-resident inflammatory foam cell macrophages were seen to be strongly associated with severity of atherosclerosis. Also, the ability to combine gene-specific programs with known risk factors can significantly strengthen the power to predict residual CVD risk.

Results

Substantial Residual CVD Risks are not Associated with Traditional Risk Factors

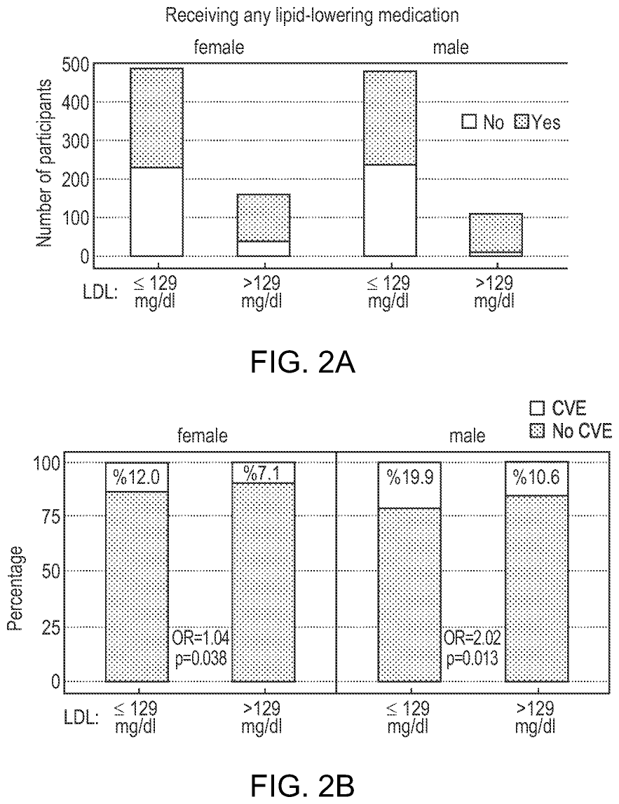

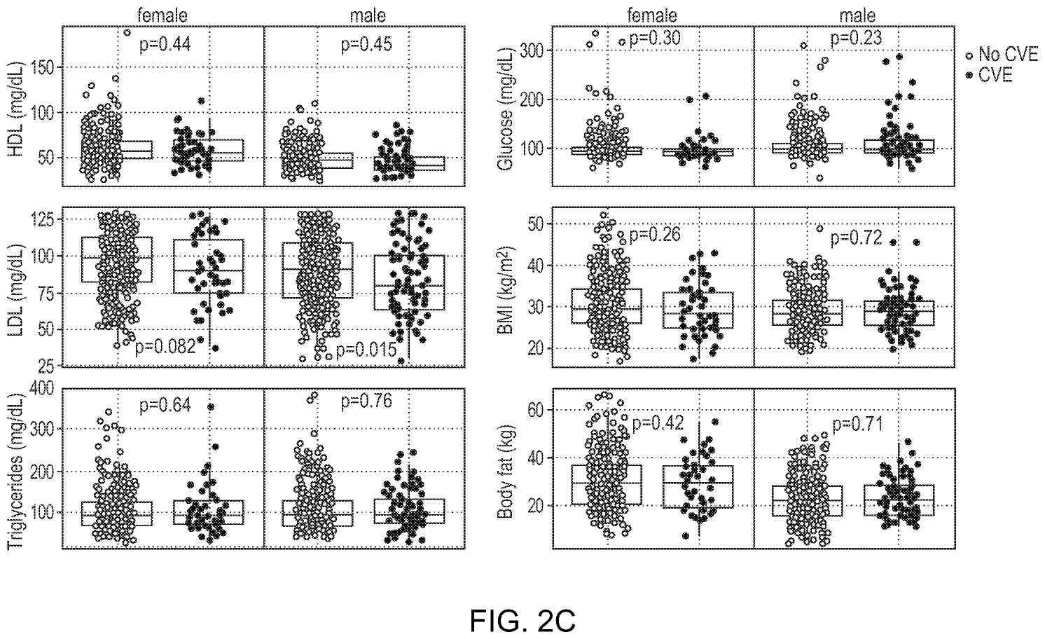

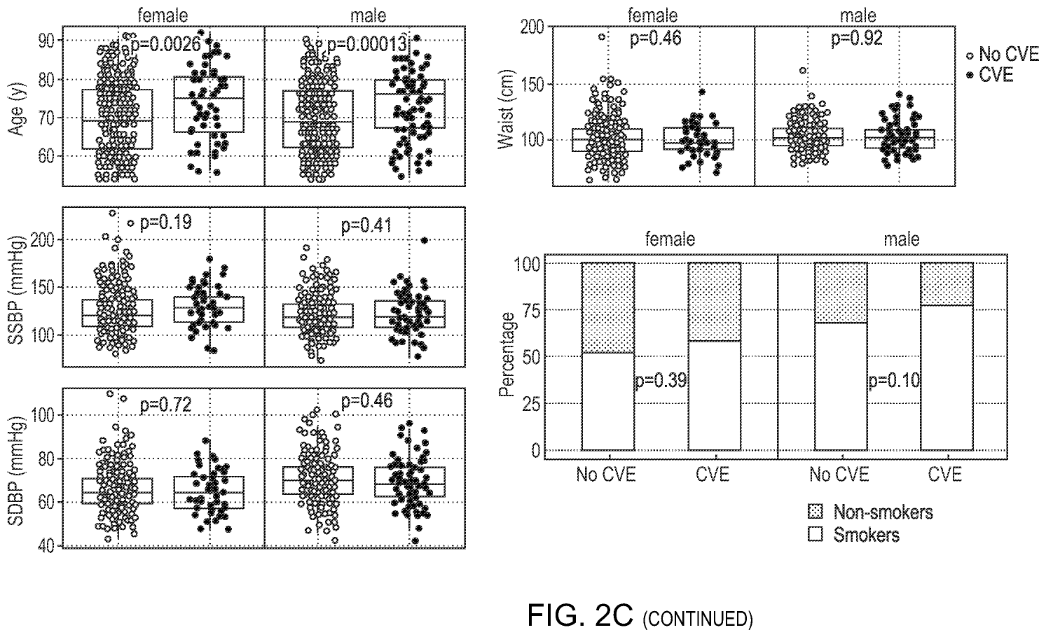

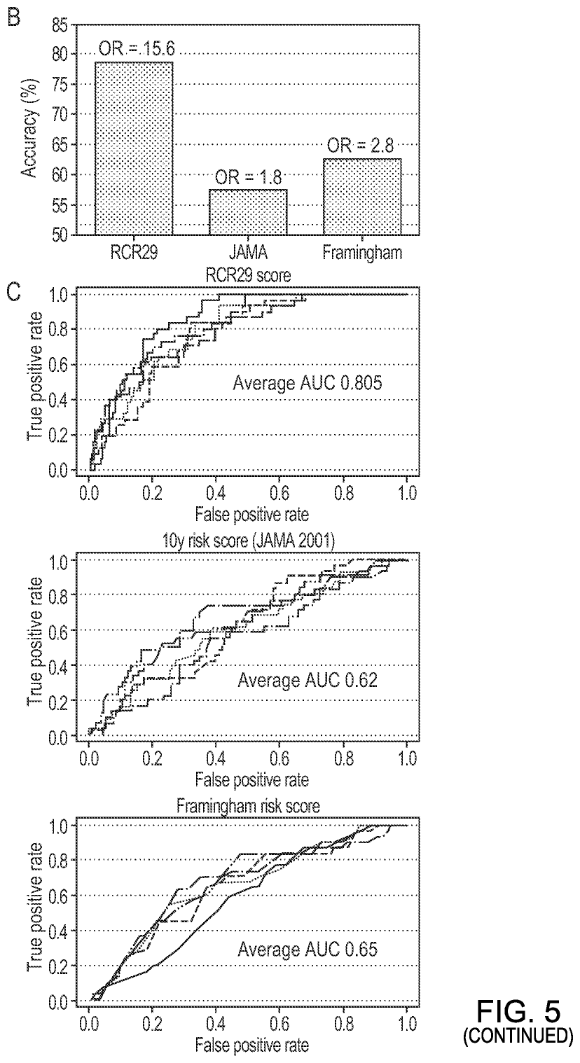

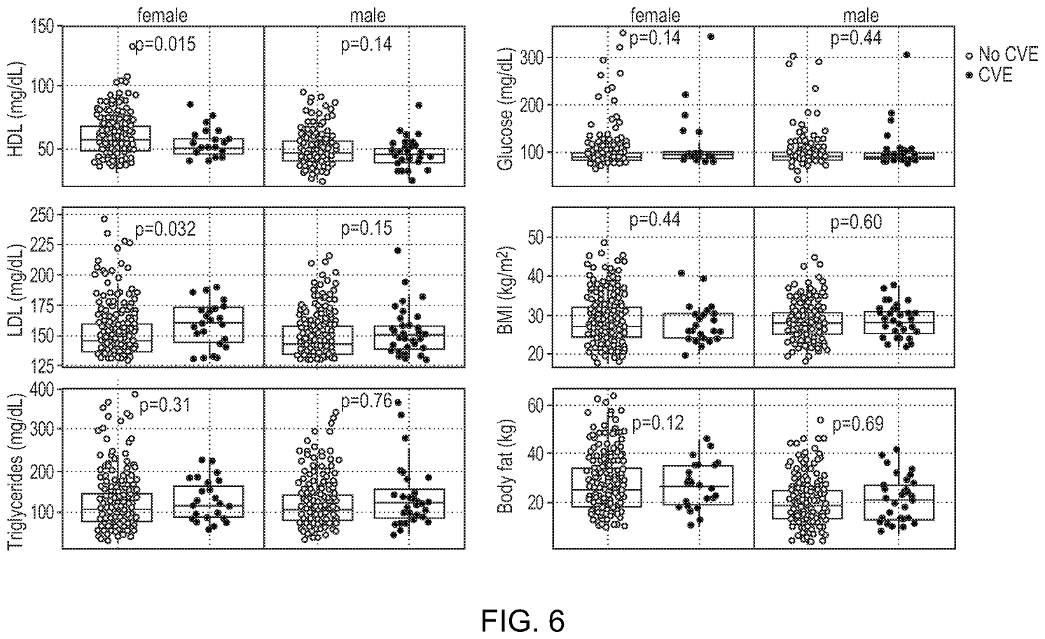

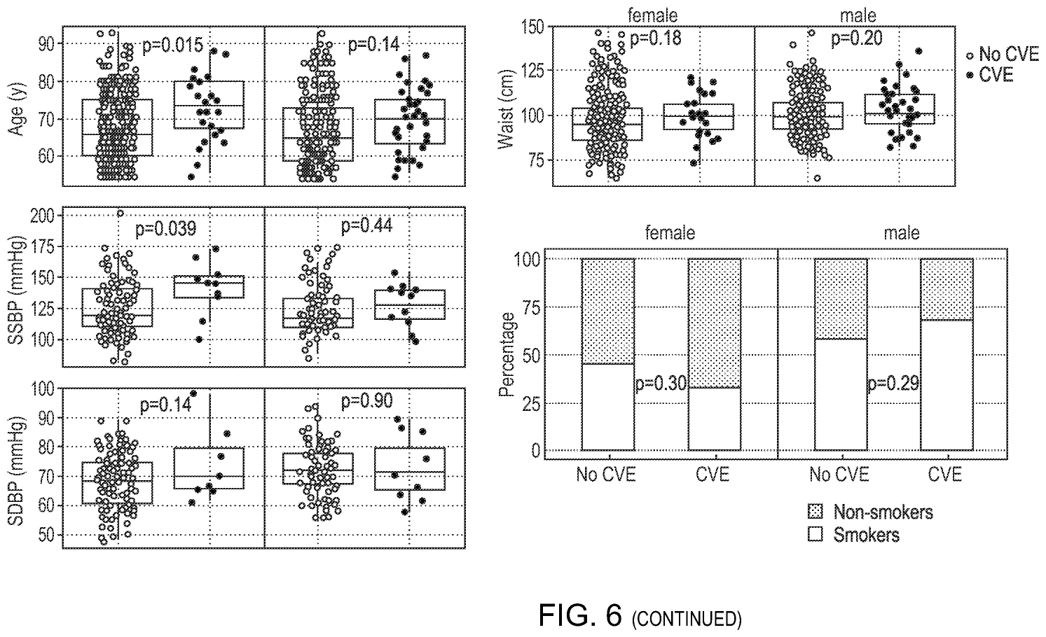

The cohort in the present study includes 1269 MESA participants consisting of 652 females and 617 males (Table 1). The majority of both females and males had LDL levels ≤129 mg/dL (Table 1), which is considered optimal or near-optimal, but may be a result of substantially applied lipid management medications (FIG. 2A) or other interventions. However, despite LDL levels being effectively managed, both female and male participants with ≤129 mg/dL LDL showed no lower risk of experiencing a CVE, and in fact showed ˜2-fold higher risk, compared to participants with >129 mg/dL LDL (OR 1.94 and 2.02, respectively; p<0.05; FIG. 2B). With the exception of age, most of the traditional risk factors including high-density lipoprotein (HDL), fasting glucose, waist circumstance, body mass index (BMI), triglycerides, body fat, blood pressure (seated diastolic blood pressure (SDBP); seated systolic blood pressure (SSBP)), and smoking status showed no significant differences between persons with or without CVE in both male or female MESA participants with S 129 mg/dL LDL (FIG. 2C). In alignment with low LDL subjects having increased CVD risk (FIG. 2B), subjects with a CVE had lower LDL levels than those without a CVE (FIG. 2C). In contrast, the group of participants with higher LDL (>129 mg/dL) exhibited changes/trends of LDL, HDL, SDBP, and SSBP that were more consistent with previous knowledge of CVD: subjects who had CVEs had lower HDL and higher LDL, and females who had CVEs had higher SSBP and SDBP (FIG. 6). These results reveal a disconnect between significantly higher CVD risks and unchanged risk factors in subjects with optimal/near-optimal LDL, and emphasize the need for novel parameters to depict their associated residual CVD risk.

TABLE 1
Characteristics of participants from the MESA cohort.

| | Female | Male |
|---|---|---|
| | N = 652 | N = 617 |
| Race | (61.4%) | (48.6%) |
| Age (years), mean ± SD | 69.5 ± 9.4 | 69.7 ± 9.3 |
| BMI (kg/m²), mean ± SD | 30.1 ± 6.2 | 29 ± 4.6 |
| Cholesterol (mg/dL), mean ± SD | 190.7 ± 34.7 | 170.1 ± 36.5 |
| LDL (mg/dL), mean ± SD | 109.5 ± 31.6 | 99.8 ± 32.5 |
| HDL (mg/dL), mean ± SD | 59.7 (16.5) | 48.3 (12.9) |
| Triglycerides (mg/dL), mean ± SD | 108.5 (54.5) | 111.9 (59.4) |
| Fasting glucose (mg/dL), mean ± SD | 100.7 (27.6) | 106.2 (29.9) |
| Body fat (kg), mean ± SD | 29.5 (11.0) | 23 (9.0) |
| Waist (cm), mean ± SD | 99.9 (15.8) | 103.2 (12.0) |
| SSBP, mean ± SD | 125.8 (21.5) | 122.2 (18.8) |
| SDBP, mean ± SD | 66.4 (9.5) | 70.7 (9.4) |
| Non-smoking, N (%) | 314 (48.5%) | 191 (31.2%) |
| Smoking, N (%) | 334 (51.5%) | 422 (68.8%) |
| LDL ≤ 129 mg/dL, N (%) | 458 (74.7%) | 478 (79.5%) |
| LDL > 129 mg/dL, N (%) | 155 (25.3%) | 123 (20.5%) |

SSBP = seated systolic blood pressure; SDBP = seated diastolic blood pressure.

AtheroSpectrum Effectively Depicts the Dynamic Transition from Macrophages to Foam Cells

Monocytes and macrophages are critical players in CVD. In the circulation, monocytes transcriptionally respond to metabolic and inflammatory cues and are recruited to sites of atherogenesis. In atherogenic sites, activities of macrophages and macrophage-derived foam cells substantially orchestrate pathology of atherosclerosis and contribute to occurrence of CVEs. Additionally, monocytes traveling through circulation that are primed by these global inflammatory and metabolic cues obtain transcriptome profiles that resemble macrophages of the pathological foaming program. Therefore, both macrophage and circulating monocyte cell populations bear comprehensive information regarding CVD risk.

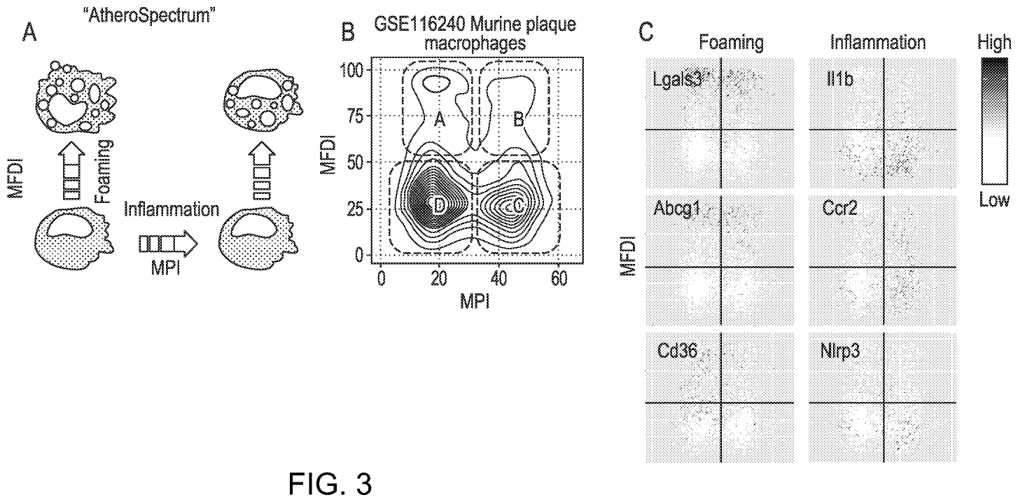

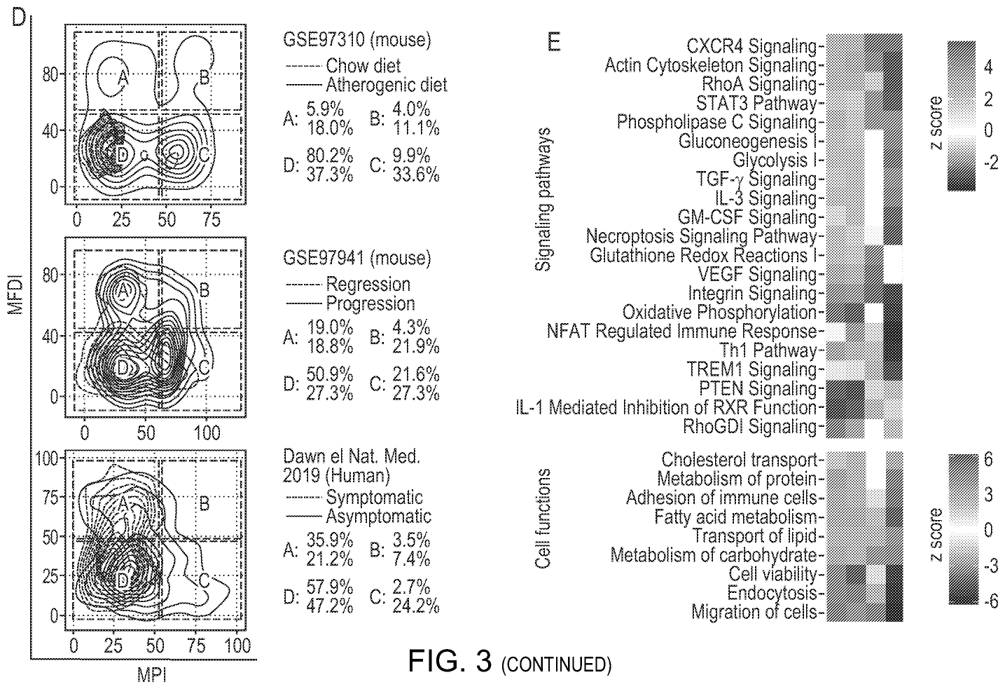

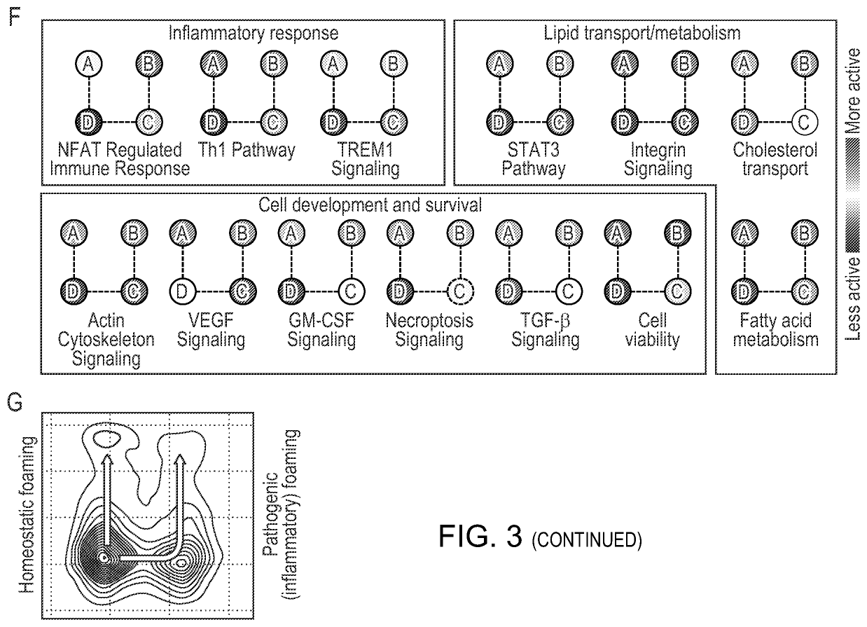

While numerous studies have indicated mixed roles of these cells in CVD pathogenesis, studying monocyte/macrophage activities is complicated by their dynamic, complex behaviors in vivo. To comprehensively characterize the diversified status of this cell lineage in the context of CVD and identify the significant cell features that associate with CVD risks, a novel computational program called AtheroSpectrum was developed. AtheroSpectrum is an extension of the recent original program MacSpectrum that was designed to fine-map monocyte/macrophage activation/inflammatory features in complex tissue settings or diseases. AtheroSpectrum was designed to capture the heterogeneity of macrophage-derived foam cells in atherogenic foci with two indices that address two key aspects of these cells: the Macrophage Polarization Index (MPI) (inherited from MacSpectrum) that describes degree of inflammation, and the novel Macrophage-Derived Foam cell Index (MDFI) that depicts foaming of macrophages (FIG. 3A). To create the MDFI, a series of calculations were performed to screen for a gene set that could effectively separate foamy and non-foamy macrophages in atherosclerotic mice (GSE116239) while retaining adequate inclusion of the most differentially expressed genes (detailed in the Methods section). Higher MPI suggests more inflammatory cells and higher MDFI suggests more differentiation towards foaming cells.

Application of AtheroSpectrum to murine atherogenic plaque macrophages (GSE116240) correctly identified foam cells, and further revealed 4 subpopulations: foaming (A), inflammatory foaming (B), inflammatory non-foaming (C), and non-inflammatory/non-foaming (D) (FIG. 3B). AtheroSpectrum accurately identified a previously reported foam cell population and its depiction was supported by expression of inflammatory and foaming marker genes in the sub-populations, including Lgals3, Abcg1, Cd36, Il1b, Ccr2, and Nlrp324 (PMID: 29122594, PMID: 30371306) (FIG. 3C). The inflammatory and foaming makers were enriched in high MPI and high MFDI cells, respectively.

To validate if similar cell sub-populations are consistently observed in atherogenesis from different studies, we applied AtheroSpectrum to several atherosclerosis datasets generated under different scenarios. In mice with on-going atherosclerosis (GSE97310)25, AtheroSpectrum identified similar distribution patterns of athero-plaque macrophages as the 4 sub-populations mentioned above (FIG. 3D, top panel). In contrast, artery macrophages from healthy, chow diet mice primarily concentrated in the “D” region, suggesting a non-inflammatory/non-foaming, unstimulated phenotype in healthy tissues. Such results suggest AtheroSpectrum effectively depicts heterogenous macrophage activation under atherogenic vs. healthy conditions, and revealed expansion of “A”, “B”, and “C” populations under atherogenic stress.

Interestingly, compared to mice exhibiting improving/regressing atherosclerosis, mice with progressing atherosclerosis (GSE97941) had markedly increased macrophage populations in regions “B” and “C”, with a similar portion in region “A” (FIG. 3D, middle panel). Similar patterns were also observed in human atherogenic macrophages (FIG. 3D, bottom panel): plaque macrophages of asymptomatic patients predominately concentrated in regions “A” and “D,” while those from symptomatic patients showed a marked increase in populations “B” and “C.” These analyses revealed that the two novel inflammatory/inflammatory-foaming populations (“C” and “B”) that were identified by AtheroSpectrum were associated with CVD severity.

Gene function analyses was further conducted on the four cell sub-populations (FIG. 3E). Many of the enriched signaling pathways and cell functions could be categorized into one of three functional groups: inflammatory response, lipid transport/metabolism, and cell development and survival (FIG. 3F). Pro-inflammatory signaling, such as the Th1 pathway, were enriched in populations “B” and “C” (FIGS. 3E-3F). Pathways of lipid metabolism (e.g., STAT3, RXR signaling pathways), lipid transport (e.g., integrin signaling, cholesterol transport, endocytosis), and cell development (e.g., GM-CSF signaling, TGF-β signaling) were more activated in populations “A” and “B” (FIGS. 3E-3F)—consistent with their predicted status as foaming macrophages. Of note, the foaming cells “A” and “B” showed both activated cell viability and necroptosis signaling, suggesting the foaming cells were proliferating and differentiating in the tissue, but died at the end of their foam cell fate. The diversified signaling in those populations revealed heterogenous functions and further supported the rational depiction by AtheroSpectrum.

Based on these analyses, two cell programs of atherogenic macrophages were proposed: the “homeostatic foaming” (“D” to “A”) that was present in both benign and severe disease conditions, and the “pathogenic foaming” (“D” to “C” to “B”) that was associated with severity of disease (FIG. 3G).

The Pathogenic Foaming Program Signature Genes Powered Depiction of Residual CVD Risks

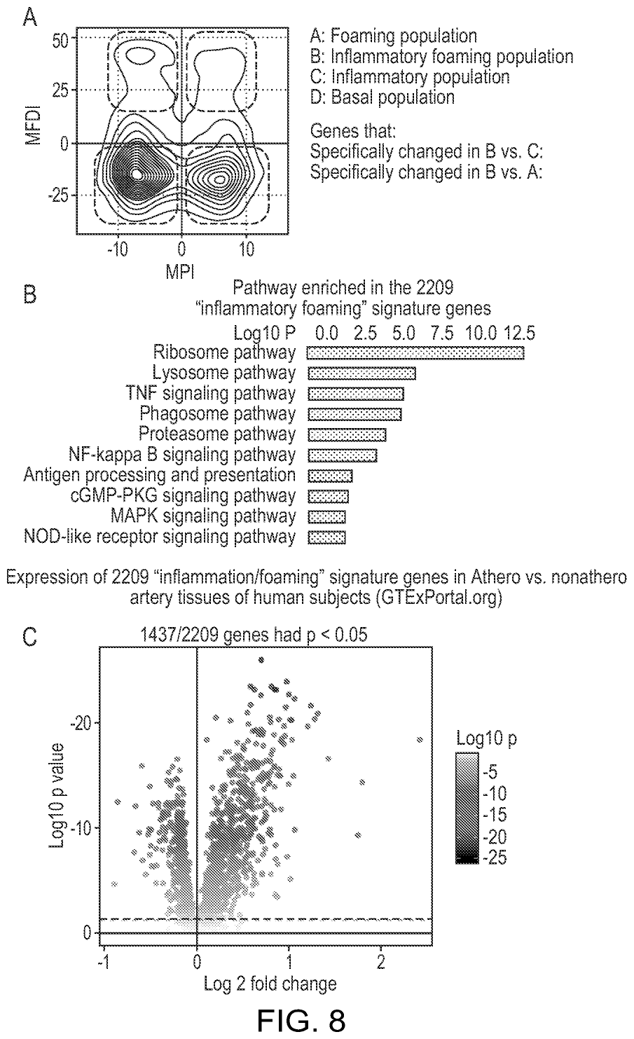

To determine the association between severity of CVD and the pathogenic foaming program, the genes specifically enriched in each program were screened. Genes that had significantly different expression in population “B” vs. “C” but not in “A” vs. “D”, and in population “B” vs. “A” but not in “C” vs. “D” resulted in a list of 2209 enriched genes that were associated with the inflammatory foaming of macrophages. Included in this gene list were signaling pathways involved in inflammation (e.g., tumor necrosis alpha [TNF-α] signaling, NF-kappa B signaling pathway), lipid transport (lysosome pathway), and lipid metabolism (MAPK signaling). A major portion of these genes (1437, 65.1%) presented significantly differential expression in athero vs. non-athero artery tissues of human subjects deposited at the GTEx Portal (gtexportal.org), supporting the pathological relevance of these genes with CVD (FIG. 8).

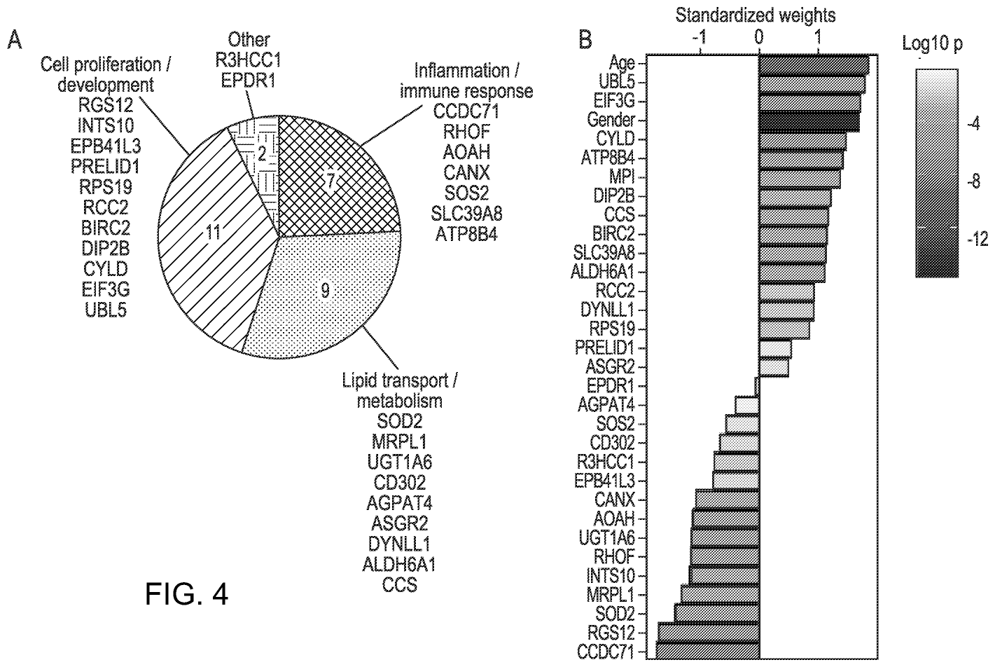

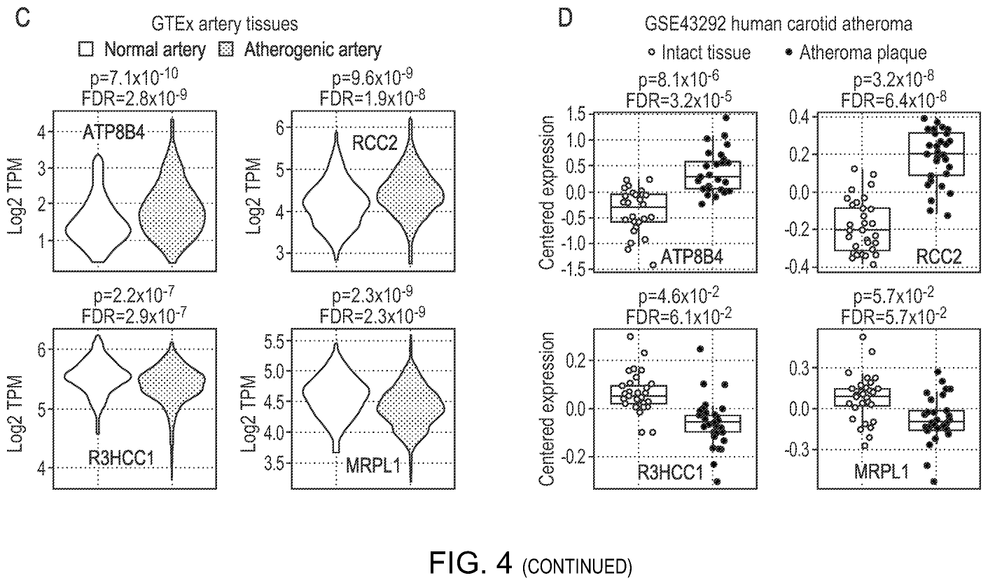

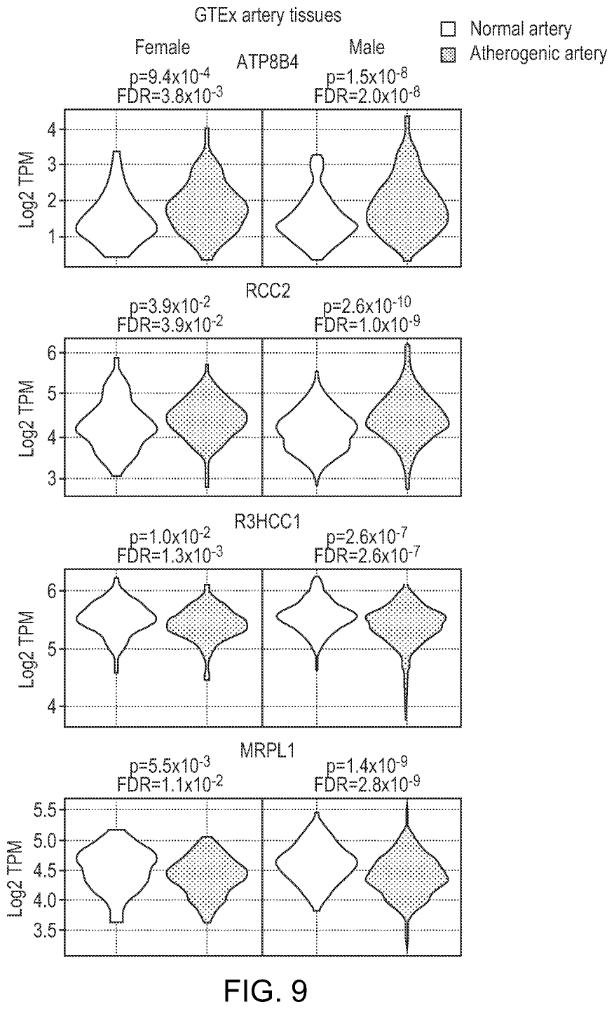

To pinpoint the signature genes that most significantly depict residual CVD risks, we further designed a self-optimizing signature gene selecting algorithm to screen the 2209 enriched genes within peripheral blood monocytes from 936 MESA participants (458 females and 478 males) of optimal/near-optimal LDL levels. This algorithm incorporated multiple steps of self-optimizing cycles that gradually narrowed down the gene list to a panel of 29 genes that most significantly depicted CVEs as evaluated by logistic regression and area under the curve (AUC) calculation (Table 2). Multivariate regression analyses showed that most of the 29 genes as well as age, gender, and MPI of participants were strongly correlated with CVEs (Table 2, FIG. 4B). Consistent with results depicted in FIG. 2, traditional risk factors—BMI, waist circumstance, fasting glucose, LDL, HDL, total cholesterol, triglyceride, or smoking—showed no significant association with residual CVD risk in this cohort. The great significance of MPI in the multivariate regression again suggested the important role of inflammation in CVD, consistent with increased inflammatory populations (“B” and “C”) in more severe CVD (FIG. 3D). Of the 29 genes, 27 of them (excluding R3HCC1 and EPDR1) could be assigned to three functional categories of inflammation/immune response, cell proliferation/development, and lipid transport/metabolism (FIG. 4A). This is also consistent with the gene functional analyses on the cell subpopulations revealed by AtheroSpectrum (FIGS. 3E-3F). Of note, ATP8B4 and RCC2, which were positively associated with occurrence of CVEs (FIG. 4B), were expressed at higher levels in athero vs. non-athero human artery tissues in the GTEx Portal (FIGS. 4C-4D) nother human carotid atheroma dataset (GSE43292), while R3HCC1 and MRPL1, which were negatively associated with occurrence of CVEs (FIG. 4B), had lower expression levels in athero vs. non-athero human artery tissues (FIGS. 4C-4D). For these genes, similar patterns were observed in both female and male subjects (FIG. 9).

TABLE 2
Multivariate regression analysis showing correlations between
CVEs and 29 CVD risk signature genes as well as MPI, MFDI,
gender, age, waist circumference, smoking, BMI, fasting glucose,
total cholesterol, LDL, HDL, triglycerides, seated diastolic
blood pressure (SDBP), and seated systolic blood pressure
(SSBP). P values calculated from multivariable logistic regression,
*p < 0.05, **p < 0.01, ***p < 0.001.

| Variable | Weight | P-value | | |
|---|---|---|---|---|
| UBL5 | 1.88249 | 1.67E−06*** | | |
| EIF3G | 1.819851 | 5.32E−08*** | | |
| ATP8B4 | 1.543647 | 8.38E−08*** | | |
| CYLD | 1.403244 | 6.22E−07*** | | |
| BIRC2 | 1.362046 | 3.01E−06*** | | |
| DIP2B | 1.231441 | 1.10E−03** | | |
| CCS | 1.159731 | 5.95E−05*** | | |
| ALDH6A1 | 1.094838 | 9.30E−05*** | | |
| DYNLL1 | 1.027766 | 2.39E−03** | | |
| RCC2 | 1.003213 | 2.49E−03** | | |
| SLC39A8 | 0.982233 | 7.11E−04*** | | |
| RPS19 | 0.745035 | 9.78E−03** | | |
| ASGR2 | 0.598124 | 2.55E−02* | | |
| PRELID1 | 0.258565 | 4.14E−01 | | |
| EPDR1 | −0.22965 | 4.09E−01 | {close oversize brace} | 29 signature |
| AGPAT4 | −0.53938 | 1.30E−01 | | genes |
| CD302 | −0.67268 | 2.58E−02* | | |
| SOS2 | −0.6985 | 3.56E−02* | | |
| R3HCC1 | −0.74783 | 5.53E−03** | | |
| EPB41L3 | −0.86342 | 1.63E−02* | | |
| CANX | −0.99678 | 9.83E−04*** | | |
| INTS10 | −1.10706 | 1.04E−04*** | | |
| UGT1A6 | −1.17414 | 6.87E−07*** | | |
| SOD2 | −1.22116 | 2.20E−04*** | | |
| RHOF | −1.28912 | 2.16E−06*** | | |
| MRPL1 | −1.31049 | 1.55E−04*** | | |
| AOAH | −1.38176 | 2.23E−07*** | | |
| CCDC71 | −1.41317 | 5.72E−06*** | | |
| RGS12 | −1.71687 | 4.82E−07*** | | |
| LDL | 15.27966 | 3.81E−01 | | |
| HDL | 10.47437 | 3.68E−01 | | |
| Triglyceride | 7.797288 | 2.93E−01 | | |
| Age | 1.95467 | 2.31E−10*** | | |
| MPI | 1.331545 | 6.31E−04*** | | |
| Sex | 1.26472 | 1.84E−08*** | | |
| SDBP | 0.485095 | 1.78E−01 | {close oversize brace} | Other |
| MFI | 0.470188 | 1.21E−01 | | parameters |
| Smoking | 0.243004 | 3.53E−01 | | |
| SSBP | −0.01597 | 9.62E−01 | | |
| Fasting glucose | −0.21794 | 3.57E−01 | | |
| BMI | −0.42502 | 3.43E−01 | | |
| Waist | −0.72123 | 9.45E−02 | | |
| Total cholesterol | −20.6723 | 3.45E−01 | | |

These results validated both statistical and pathological relevance of the 29-gene panel with CVEs in subjects with optimal/near-optimal LDL.

Novel Residual CVD Risk Score by 29 Genes (RCR29) Effectively Depicted Residual CVD Risks

To facilitate the depiction of residual CVD risks, we developed a novel residual CVD risk score, RCR29, incorporating the 29-gene panel screened by this machine learning algorithm with subject age, gender, and MPI. The score ranges from 0-10, with 0 and 10 indicating lowest and highest risk for a CVE, respectively.

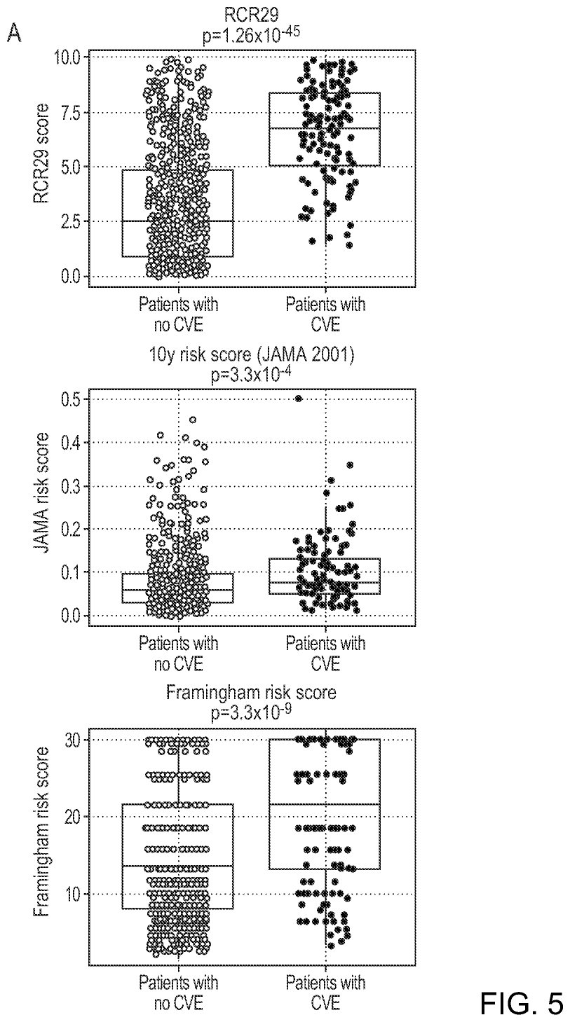

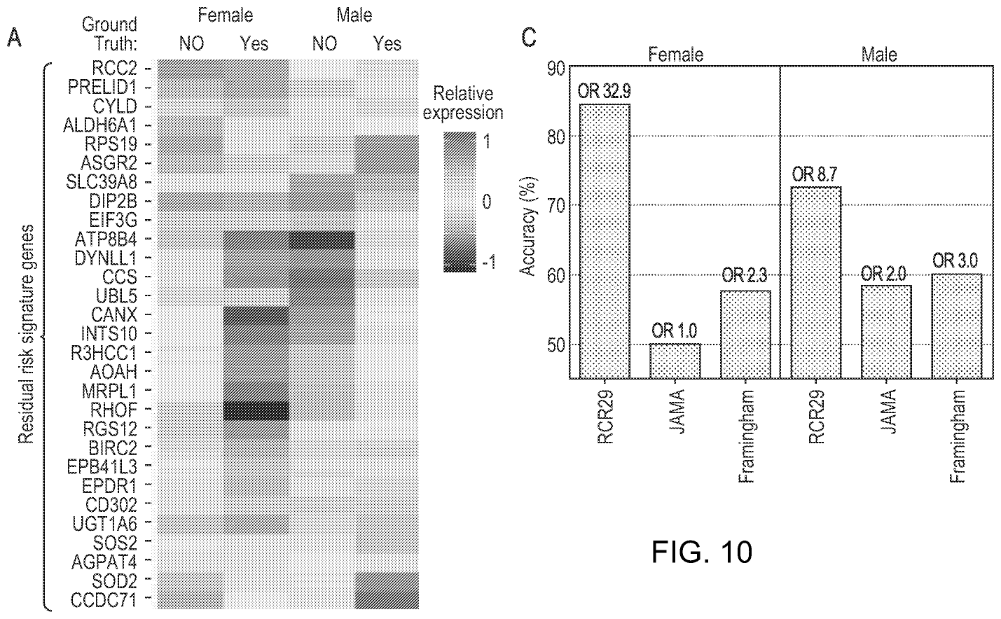

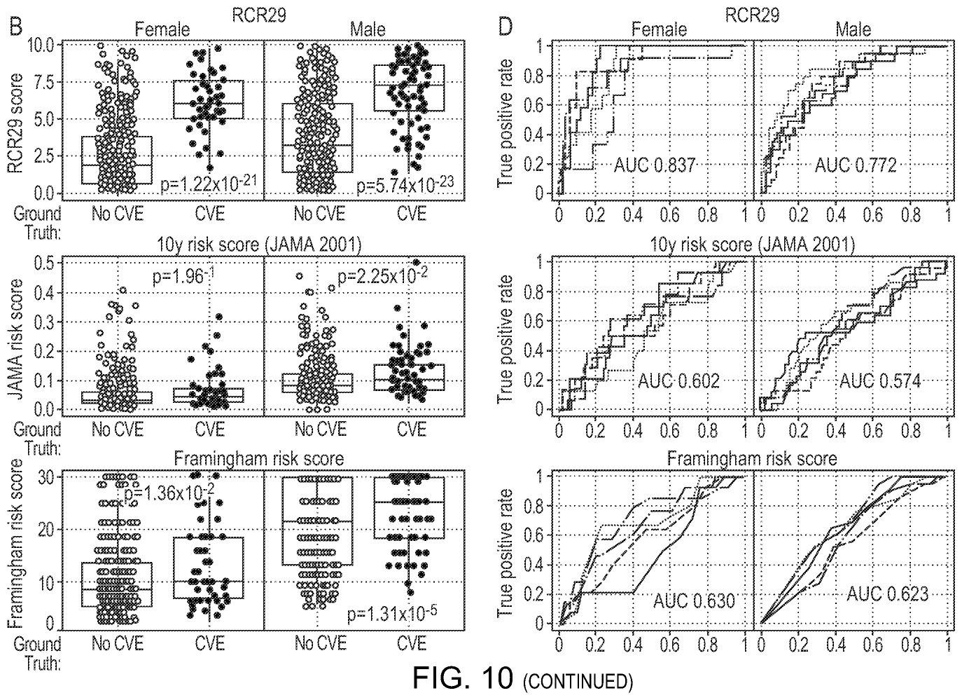

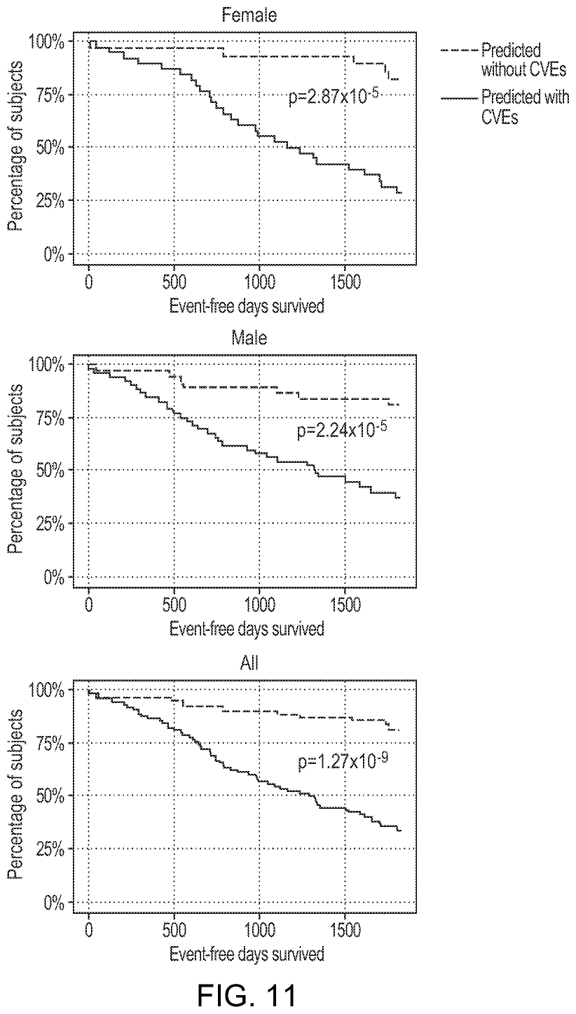

Subjects with optimal/near-optimal LDL and CVEs had significantly higher residual CVD risk scores compared to those without CVEs (FIG. 5A). Using manually selected thresholds, RCR29 showed 79% accuracy in predicting CVEs (OR=15.6), which is markedly higher than the JAMA29 (58%) and Framingham risk scores (63%) for the same cohort (FIG. 5B). Consistently, the ROC curve of residual CVD risk score as a predictor reached AUC of 0.805 which is also higher than both the JAMA and Framingham scores (FIG. 5C). The better performance of RCR29 over the other scores was consistently observed in both females and males. Interestingly, RCR29 had higher predicting power and accuracy in females than males (FIGS. 10B-10D), and the 29 residual risk signature genes showed differential expression patterns between the two sexes (FIG. 10A). In addition, male and female MESA subjects who were predicted to have a CVE by their RCR29 score showed significantly shorter event free survival days since the monocyte collection time than subjects predicted to have no events (FIG. 11). This RCR29 score powered by AtheroSpectrum and a novel machine learning algorithm effectively depicted residual CVD risks in the MESA cohort, exhibiting strong potential for predicting future CVEs.

Discussion

Risk assessment is critical for determining appropriate interventions and minimizing occurrence of future CVEs. For decades, traditional cardiovascular risk factors, such as HDL, LDL, and triglyceride levels have been targeted in CVD patients and/or high-risk groups for preventative interventions. However, a substantial number of people with traditional risk factors largely under control may still experience new CVEs, such as heart attack or stroke. These data reveal a clear disconnect between traditional risk factors and CVEs in the subjects who had well-managed lipid levels but have residual CVD risks. The majority of these subjects were of optimal/near optimal LDL levels, and most of them had normal ranges of HDL (≥40 mg/dL), triglyceride (<150 mg/dL), and fasting glucose (<100 mg/dL). Importantly, all such factors—as well as BMI, waist circumstance, body fat, and smoking status-showed no difference between subjects who experienced CVEs or not. Therefore, these factors are no longer suitable for assessing risk of future CVEs in subjects with optimal/near optimal LDL levels.

The monocyte-macrophage lineage plays a critical role in the development of CVD. Monocytes sense cues from the microenvironment as they travel through circulation and adopt specific gene expression profiles in response. Upon recruitment to the artery intima in atherogenic conditions, monocytes differentiate into macrophages and orchestrate pathological progression depending on their activation and foaming process. Therefore, this cell lineage carries comprehensive information on subjects with atherogenic conditions, making them ideal targets for analyzing CVD risk. However, due to a lack of high-resolution approaches, previous studies of this lineage only found limited if any association with CVEs, and monocyte/macrophage-derived predictive factors for residual CVD risk was never investigated.

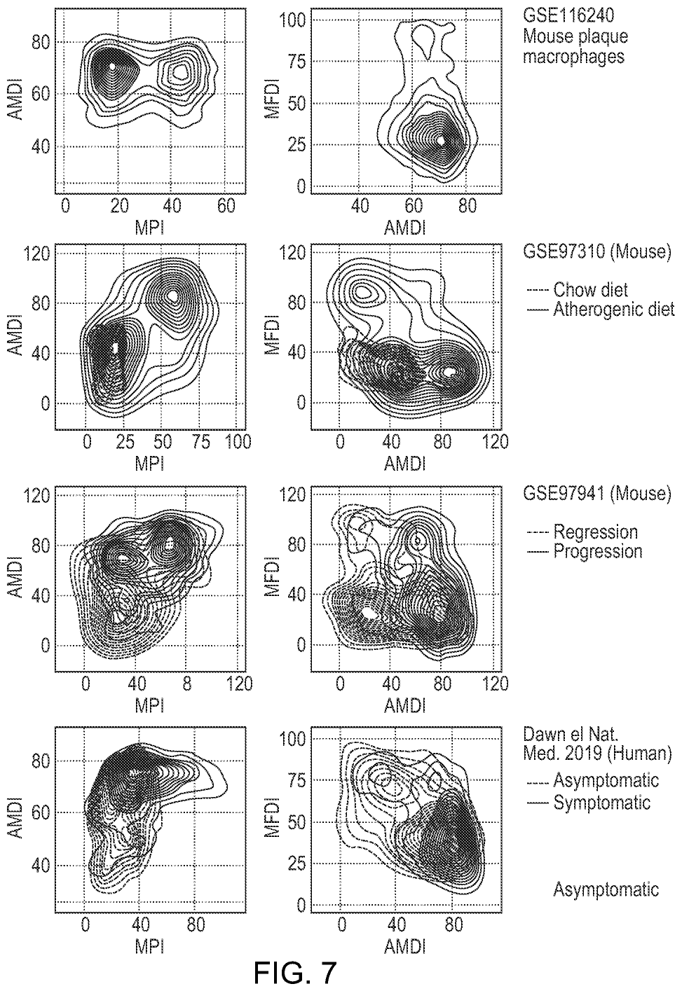

Herein, a novel system called AtheroSpectrum was developed to profile foaming macrophages during atherogenesis, based on the previously released macrophage analysis platform, MacSpectrum. This system features two indices, MPI and MDFI that depict the inflammation and foaming of atherogenic macrophages, respectively. The performance of AtheroSpectrum was validated with multiple datasets from separate studies (FIG. 3D). In murine plaque macrophages (GSE116240), AtheroSpectrum accurately depicted the foam cell population, which was indicated by expression of foam cell signature genes Lgals3 and Abcg1 reported in the original publication (FIG. 3C). In another study (GSE97310)25, AtheroSpectrum revealed expansion of foaming and inflammatory macrophage populations in atherogenic diet-induced atherosclerotic mice compared to chow diet mice (FIG. 3D), which was consistent with previous knowledge of atherogenic macrophages. In contrast, the Activation-induced Macrophage Differentiation Index (AMDI) of MacSpectrum did not consistently depict heterogeneous populations of artery macrophages in atherosclerosis (FIG. 7), as this index was designed to address terminal maturation of macrophages, which may or may not be present in the case of atherogenic macrophages. These results support the efficacy of AtheroSpectrum, which is tailored for this disease.

AtheroSpectrum not only correctly identified known features of atherogenic macrophages but also identified a novel program consisting of both inflammatory and foaming cell populations that were consistently higher in more severe CVD conditions: increased inflammatory/foaming populations in mice with progressing vs. regressing atherosclerosis, and in human subjects of symptomatic vs. asymptomatic atherosclerosis (FIG. 3D). For the first time, these discoveries suggest that not all foam cells contribute to atherogenesis equally, and inflammatory foaming—which was termed “pathological foaming”—is associated with detrimental outcomes.

Next the 2209 genes were extracted that were enriched in the pathological foaming program of atherogenic macrophages. Since monocytes travel through circulation and are primed by global inflammation and metabolism cues, monocyte transcriptome profiles that resembled macrophages of the pathological foaming program might suggest a more detrimental internal environment that may favor occurrence of CVEs. Therefore, the 2209 genes were further screened in circulating-monocyte transcriptome profiles of the MESA cohort for association with CVEs. Genes form complex networks and have substantial interactions, so significant association with CVEs is more likely to exist for a certain set of genes rather than individual genes. However, even within the 2209 enriched gene pool, there could be enormous combinations of genes (e.g., taking 29 from 2209 genes can produce 9.0×10⁶⁵ different gene sets), and it is impossible to screen each of them in a feasible time course. To solve this challenge, we created a novel algorithm that features recursive random sampling and self-optimizing; through 5 stages of screening, our algorithm gradually narrowed down a candidate gene list and eventually produced a final signature gene set of 29 genes that had strong association with occurrence of CVEs. Most of the 29 genes were annotated with one of three functional categories: inflammation/immune response, lipid transport/metabolism, and cell proliferation/development (FIG. 4A), suggesting our algorithm indeed identified functionally interrelated genes.

In addition to the 29 signature genes, the MPI of monocytes also showed significant association with CVEs (Table 2), suggesting an important role of inflammation for the increased pathological foaming program in more severe CVD conditions (FIG. 3D). In contrast, the lipid levels showed no significant association with CVEs in subjects of optimal/near-optimal LDL (Table 2), suggesting those traditional factors are not capable of assessing residual CVD risk in this cohort.

Based on the 29 signature genes as well as MPI, gender, and age, we developed a novel score system, named RCR29 (Residual CVD Risk score by 29 genes), for assessing CVD residual risk by monocyte gene expression profiles. In the MESA cohort of this study, RCR29 reached an AUC of 0.802 and OR of 15.6 (with threshold set at 5.0), suggesting much higher predicting power of residual risk than the Framingham and JAMA risk scores (FIG. 5). This is not surprising considering that the latter two relied on traditional risk factors, including lipid levels and smoking, which show very limited association with CVEs in the optimal/near-optimal LDL subjects (FIG. 2C, Table 2). This work established the potential for application as an easy-to-conduct and less-invasive approach for assessing residual CVD risk with monocytes from peripheral blood, and therefore would be feasible for longitudinal study and the follow-up evaluation of CVD medications. Limited by the size of available dataset, our current RCR29 score has not addressed each ethnic group specifically, which was due to the numbers of subjects with/without CVE in each ethnic group that were too small to provide enough statistical power (Table 3). When more datasets with CVEs and detailed time courses become available, our RCR29 score can be further improved to provide more precise and robust prediction of residual CVD risks within specified periods of time.

TABLE 3
Ethnic distribution of participants from the MESA cohort.

| | Female | | Male | |
|---|---|---|---|---|
| | No CVE | CVE | No CVE | CVE |
| White, Caucasian | 248 | 35 | 249 | 51 |
| Black, African-American | 130 | 8 | 91 | 15 |
| Hispanic | 169 | 23 | 153 | 42 |

CVE = Cardiovascular event

Methods

Participants

As previously reported 20, the present analyses are primarily based on data collected at MESA Exam (April 2010-February 2012) with concurrent analyses of purified monocyte samples of 1264 randomly selected MESA participants from four MESA sites (John Hopkins University, Columbia University, University of Minnesota, and Wake Forest University). The study protocol was approved by the Institutional Review Boards of the four institutions. All participants signed informed consent.

Measurements of Participants

All measurements and records of CVEs in humans were obtained at MESA Exam 520. Of the 1264 selected participants, 1206 had valid records of CVEs.

Transcriptome Analysis of Blood Monocytes

Blood monocytes of MESA participants were isolated and profiled by microarray as previously reported. Blood was collected in sodium heparin-containing Vacutainer CPT cell separation tubes (Becton Dickinson, Rutherford, NJ), and subsequently, monocytes were isolated with anti-CD14 antibody-coated magnetic beads, using an autoMACS automated magnetic separation unit (MiltenyiBiotec, Bergisch Gladbach, Germany). Purity of monocytes was validated using flow cytometry, which was >90%. RNA was isolated from samples using the AllPrep DNA/RNA Mini Kit (Qiagen, Inc., Hilden, Germany). The median of RNA Integrity (RIN) scores for the 1264 RNA samples was 9.9. RNAs with RIN<9.0 were excluded from global expression microarrays. Transcriptomes of the monocytes were profiled using the Illumina HumanHT-12 v4 Expression BeadChip and Illumina Bead Array Reader.

Screening of Foaming Signature Genes (FSG)

AtheroSpectrum consists of two indices, the Macrophage Polarization Index (MPI) and the Macrophage-Derived Foam cell Index (MDFI). MPI was inherited from MacSpectrum and was calculated as previously described 23; the only difference is that in AtheroSpectrum the MPI was scaled to 0 to 100, instead of −50 to 50 in MacSpectrum. To create MDFI, 500 FSG were first screened. For identification of FSG, differential expression of genes in macrophages from atherosclerotic plaques of atherogenic diet mice (n=3) vs. artery macrophages from chow diet mice (n=3) (GSE116239) was conducted. From the 1118 genes that were significantly changed (p<0.05) between atherogenic and chow diet mice, we excluded any genes in the previously reported 500 polarization signature genes (PSG) or 435 activation-induced macrophage differentiation signature genes (AMDSG), as those genes are also involved in macrophage inflammation or activation-induced differentiation (e.g., maturation from monocyte to macrophage). From the remaining 759 genes, the top 500 were taken as the FSG. The 500 FSG represented the most significantly changed genes in the aspect of macrophage foaming.

Macrophage Transcriptome Profile Similarity to Atherosclerosis or Healthy Reference Populations

Two sets of reference transcriptome profiles were generated as the average Reads Per Kilobase of transcript, per Million mapped reads (RPKM) of each FSG gene in artery macrophages from atherosclerosis plaques of atherogenic diet mice (n=3) or chow diet mice (n=3) (GSE116239), and termed the chow-average reference (Chow_{ref}) and the athero-average reference (Athero_{ref}). Similarities between a macrophage transcriptome profile and the two references were calculated similarly as previously reported with R using a method modified from Pearson's correlation (r_{chow} and rₐₜₕₑᵣₒ, equations listed below).

The FSG were first adapted to the macrophage transcriptomes, resulting in (sub)sets of FSG (FSG_{sub}) that were expressed in the macrophages to be computed. To emphasize the changes in gene expression during macrophage foaming under various conditions, a gene set centering adjustment were performed to focus on fold-change difference of each individual gene in all samples within a study. The average expression level of a given gene (S_{avg}) was calculated as the mean of this gene in each dataset (S) in the whole study. S_{ct} is calculated as the centered value of this gene: let S be the original expression level, then the centered expression S_{ctr} will be:

S_{ctr}=S−S_{avg}

Accordingly, the two references, Chow_{ref} and Athero_{ref}, were adjusted by centering each gene expression level as described, and termed Chow_{ctr} and Athero_{ctr}. The similarities between a macrophage transcriptome profile (gene expression levels in RPKM/FPKM/UMI, etc.) and the centered references were calculated as the following: i represents a gene ID among the FSG set; n=number of genes in FSG_{sub}.

$${r}_{chow} = \frac{{\sum }_{i = 1}^{n} {S}_{ct i} \times {Chow}_{ctr i}}{\sqrt{{\sum }_{i = 1}^{n} {S}_{ctr i}^{2}} \sqrt{{\sum }_{i = 1}^{n} {Chow}_{ctr i}^{2}}}$$

$$And$$

$${r}_{athero} = \frac{{\sum }_{i = 1}^{n} {S}_{ct i} \times {Ahero}_{crt i}}{\sqrt{{\sum }_{i = 1}^{n} {S}_{ctr i}^{2}} \sqrt{{\sum }_{i = 1}^{n} {Athero}_{ctr i}^{2}}}$$

Adjusted correlation values for each comparison is provided as r_{chow} and rₐₜₕₑᵣₒ for each macrophage transcriptome profile. Each cell in a single-cell RNA sequencing (scRNA-seq) data set or an RNA-seq profile in a given experiment was treated as one transcriptome profile and calculated for the two r values.

Creation of MFDI for Each Cell

MFDI was created using a similar method as MPI23. The rchow and rathero values of three atherosclerosis macrophage transcriptome profiles (atherogenesis diet mice) and three normal artery macrophages profiles (chow diet mice)24 were calculated as mentioned above. Linear regression was performed on the macrophage profiles on the rm1-rm2 plot, producing an adjusted R2 value of 1.000. The regression line was defined as the foaming axis. Let the equation of the regression line be: ax+by +c=0. The coordinates of each sample's projection on the regression line (P) were calculated as follows:

$${x}_{p} = \frac{{b}^{2} {r}_{chow} - {abr}_{athero} - ac}{{a}^{2} + {b}^{2}}$$

$${y}_{p} = \frac{{a}^{2} {r}_{athero} - {abr}_{chow} - bc}{{a}^{2} + {b}^{2}}$$

The correlation r values were always within the range −1 to 1; we then set the left-most point on the plot

$${P}_{0} ( - 1 , \frac{a - c}{b} )$$

as the reference zero-point. Accordingly, the right-most point Pmax

$$( 1 , - \frac{a - c}{b} )$$

was set as the reference Pₘₐₓ point. The distance l from the reference 0 point to a given macrophage sample's P (xₚ, yₚ) was calculated as the following:

l=\sqrt{(xₚ−xₚ₀)²+(yₚ−yₚ₀)²}

The distance between Pₘₐₓ and P₀ was scaled to create a 0 (P₀) to 100 (Pₘₐₓ) range and the [symbol] value of each macrophage sample was rescaled accordingly, resulting in its MFDI.

Multivariable Regression

Gene expression levels and continuous features (LDL, fasting glucose, age, MPI, etc.) of MESA subjects were rescaled as the following: for each gene or feature, the 5^{th} percentile was set as 0; the 95^{th} percentile was set as 1, all other values were linearly rescaled accordingly. This was to avoid biased weights in the multivariable regression caused by different expression levels of genes and different scales of features. Binary features (e.g., female/male, non-smoker/smoker, non-CVE/CVE) were coded as 0 and 1.

Multivariable logistic regression was performed between CVEs and the gene-set being investigated plus MPI and selected MESA features (gender, age, HDL, LDL, triglyceride, fasting glucose, smoking, etc.) using R.

5-Fold Cross Validation

A 5-fold cross validation was conducted for unbiased evaluation of model performance. We first randomly shuffled the subjects in the MESA cohort and divided the subjects into 5 equal groups. Then, the first group was used for logistic regression model testing, and the other 4 groups for model training; next, the second group was used for model testing, and the rest for training; we kept running this procedure until each of the 5 groups was used for testing once. A receiver operating characteristic (ROC) curve was plotted and area under curve (AUC) was calculated for each group used for testing. Overall performance of the model was presented as average AUC of the 5 groups.

Machine Learning-Powered CVD Residual Risk Signature Gene Identification

To identify CVD residual risk signature genes from the 2209 genes enriched in the pathological foaming program, we designed a 7-step, self-optimizing machine learning procedure as follows: Every step consists of 20 batches. Step 1: For each batch, we randomly selected 5,000 gene sets from the 2209 gene pool; each gene set contained 25-30 genes. Multivariable logistic regression modeling was conducted using each gene set plus MPI and selected MESA features, and the gene set that produced the highest AUC in model testing was kept as the candidate gene set of this batch. 15 out of the 20 batch candidate gene sets that had the highest AUCs were selected and pooled, which was used as the gene pool in the next step.

Step 2: Same as step 1, except that the outcome of step 1 was used as the gene pool, and the top 12 of 20 batch candidate gene sets were pooled and used for step 3.

Steps 3-7 were conducted using the same procedures, and the top 8, 5, 3, 2, 1 of the candidate gene sets were selected and pooled at the end of each step, respectively.

The outcome gene set of step 7 was the final CVD residual risk signature genes identified by this machine learning procedure, which included 29 genes.

Creation of the RCR29 Score

The logistic regression model was trained using the CVD residual risk signature genes as well as gender, age, and MPI. Each MESA subject was input into the model, generating a regression value between 0 and 1 representing their probability of having CVEs (0-no event; 1=event). The regression values were linearly rescaled to a 0-10 range, and reported as the RCR29 score.

Signaling Enrichment Analyses

Signaling pathway and cell function enrichment were analyzed using Ingenuity Pathway Analysis (IPA) (Qiagen) and NIH DAVID Bioinformatics Resources (david-d.ncifcrf.gov).

CVE-Free Survival Analyses

Cox proportional hazards regression was conducted on 33 female and 45 male MESA subjects who had their CVE after their monocyte collection date, as well as equal numbers of randomly selected subjects who did not have a CVE. The analyses were done using R and the packages “survival” (github.com/therneau/survival) and “survminer” (rpkgs.datanovia.com/survminer/index.html).

Statistics

Unless otherwise stated, P values of gene differential expression were determined by 2-tailed Welch's t-test. P-values of enrichment of pathways, upstream regulators, and gene ontology terms were generated by the corresponding bioinformatics tools. All statistics calculations were conducted using R unless otherwise stated.

Data Availability

Monocyte transcriptome profiles of MESA participants were deposited in the NCBI Gene Expression Omnibus and are accessible through GEO Series accession number (GSE56047)20. The other datasets used in this study were accessed with the GEO Series accession number GSE9731025 and GSE9794126, or from Figshare (figshare.com/s/c00d88b1b25efDc5c788, DOI:10.6084/m9.figshare.9206387).

### Example 2: Single Cell Transcriptomics-Based MacSpectrum Reveals Novel Macrophage Activation Signatures in Diseases

Adipose tissue macrophages (ATMs) are crucial for maintaining adipose tissue homeostasis and mediating obesity-induced metabolic abnormalities, including prediabetic conditions and type 2 diabetes mellitus. Despite their key functions in regulating adipose tissue metabolic and immunologic homeostasis under normal and obese conditions, a high-resolution transcriptome annotation system that can capture ATM multifaceted activation profiles has not yet been developed. This is primarily attributed to the complexity of their differentiation/activation process in adipose tissue and their diverse activation profiles in response to microenvironmental cues. Although the concept of multifaceted macrophage action is well accepted, no current model precisely depicts their dynamically regulated in vivo features. To address this knowledge gap, single-cell transcriptome data was generated from primary bone marrow-derived macrophages under polarizing and nonpolarizing conditions to develop new high-resolution algorithms. The outcome was the creation of a 2-index platform, MacSpectrum (macspectrum.uconn.edu), that enables comprehensive high-resolution mapping of macrophage activation states from diverse mixed cell populations. MacSpectrum captured dynamic transitions of macrophage subpopulations under both in vitro and in vivo conditions. Importantly, MacSpectrum revealed unique signature gene sets in ATMs and circulating monocytes that displayed significant correlation with BMI and homeostasis model assessment of insulin resistance (HOMA-IR) in obese human patients. Thus, MacSpectrum provides unprecedented resolution to decode macrophage heterogeneity and will open new areas of clinical translation.

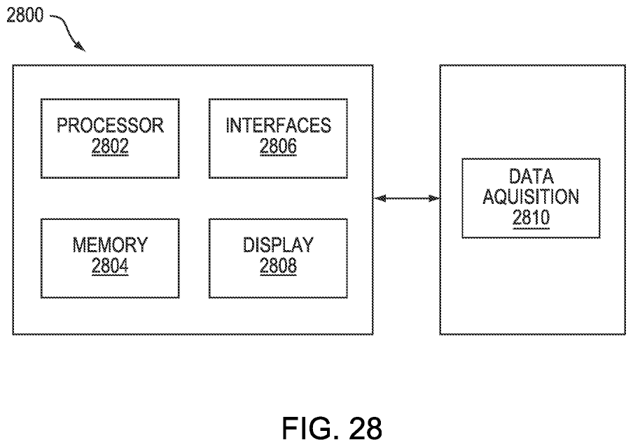

TABLE B
Terms of art.

| Term | Abbreviation | Description |
|---|---|---|
| Terminal | | The transitional process from preactivation-state |
| maturation | | macrophages to fully activated/polarized macrophages. |
| Polarization | PSG | The genes used for MPI generation; they were selected by |
| signature genes | | comparing M1 vs. M2 BMDMs. |
| Polarization | | The axis along the regression line of M1 and M2 BMDMs |
| axis | | (FIG. 28), pointing from the most M2-like to the most |
| | | M1-like cells; MPI was determined based on a cell's projectior |
| | | on the polarization axis. |
| Macrophage | MPI | The index describing the polarity of macrophage activation, |
| polarízation | | the lowest and highest MPI values suggest the most M2- and |
| index | | M1-like macrophages in a population, respectively. |
| Activation-induced | AMDSG | The genes used for AMDI generation; they were selected |
| macrophage differentiation | | by comparing M0 vs. M1 and M2 BMDMs. |
| signature genes | | |
| Activation-induced | AMDI | The index describing the degree of macrophage terminal |
| macrophage differentiation | | maturation. |
| index | | |

Results

Conventional Analysis of Single-Cell RNA Sequencing Data Revealed Nonoverlapping Cell Cluster Distribution Between Primary ATMs and the In Vitro Bone Marrow-Derived Macrophage Polarization System.

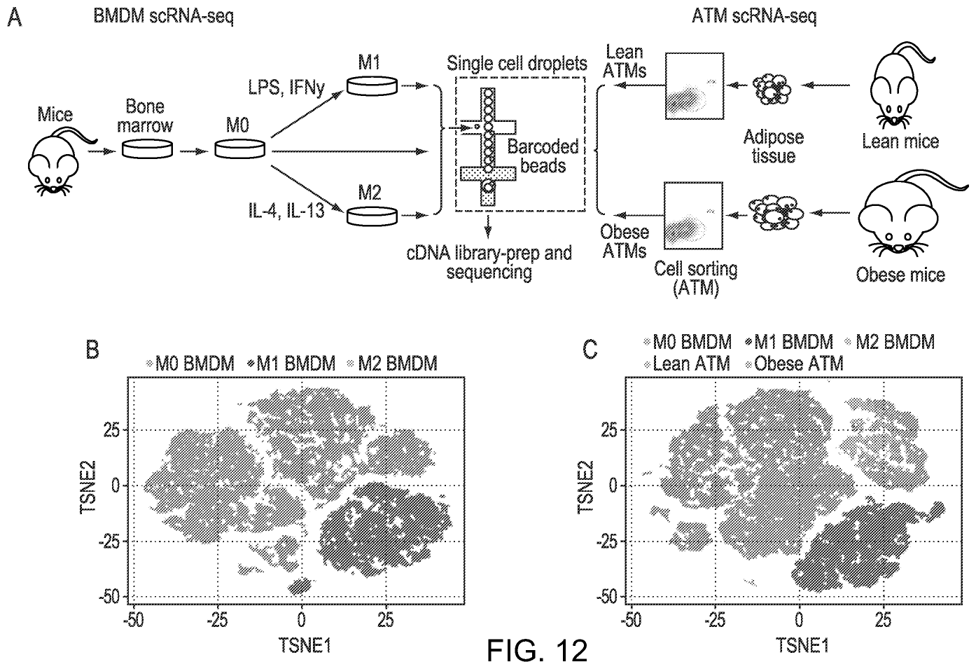

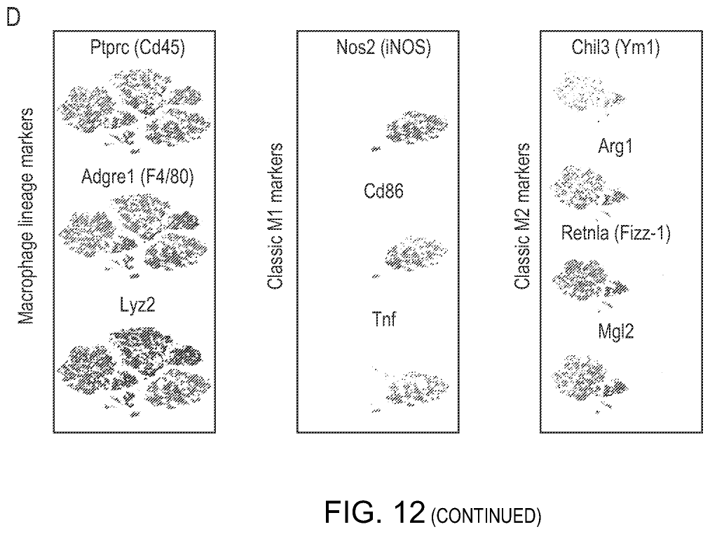

ATMs are the most abundant immune population in white visceral fat and are known to play crucial roles in maintaining adipose tissue function and immunological homeostasis. ATMs increase significantly in quantity and proportion in obese visceral fat, while the whole ATM population has been shown to shift from a less inflammatory M2-like activation state to a proinflammatory M1-like activation state. Nevertheless, despite transcriptome profiles of ATMs previously documented in different studies, no known transcriptome annotation tool allows full-spectrum capture of the dynamic action of macrophages in vivo or depiction of the dynamic yet distinct activation programs of ATMs under healthy or obese conditions. To fill this knowledge gap, single-cell RNA sequencing (scRNA-seq) analyses of purified ATMs (CD45⁺CD11b⁺F4/80⁺) from diet-induced obese or normal chow diet-fed lean mice was performed. In parallel, to facilitate characterization of ATM subpopulations with respect to activation and terminal differentiation states, we also generated scRNA-seq profiles of the in vitro polarization system, a well-established model where primary bone marrow-derived macrophages (BMDMs) are stimulated with robust polarization conditions: unstimulated for M0, IFN-γ plus LPS for M1, and IL-4 plus IL-13 for M2 (FIG. 12A).

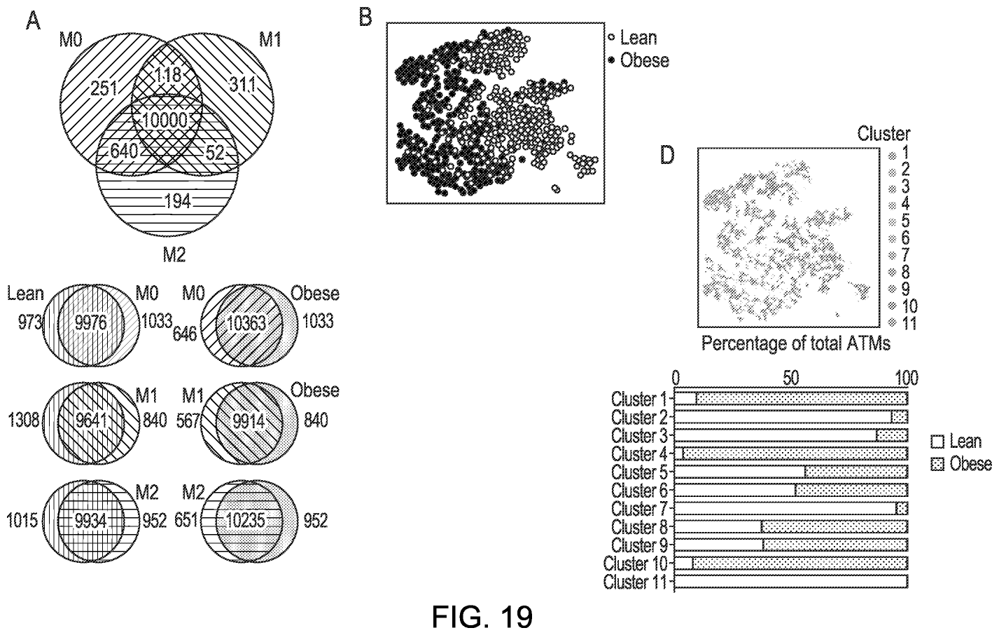

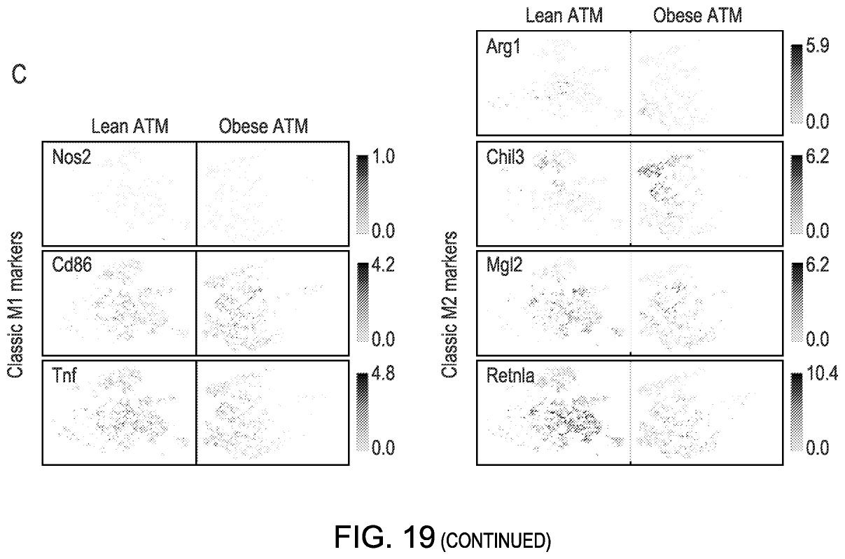

scRNA-seq profiles were analyzed using CellRanger, which was developed using the widely accepted t-distributed stochastic neighbor embedding (t-SNE) algorithm, to focus on similarity comparisons at the whole-transcriptome level. Three distinct BMDM cell clusters were identified using t-SNE that were consistent with the anticipated treatments in culture (FIG. 12B), and were validated by the presence of mature macrophage signature genes (CD45, Adgre1 [F4/80], Lyz2) and their polarization signature gene expression patterns (e.g., Nos2, CD86, and Tnf for M1; Ym1, Arg1, Fizz-1, and Mg/2 for M2) (FIG. 12D). However, when this strategy was applied using scRNA-seq profiles from ATMs isolated from obese or lean murine visceral adipose tissue, our t-SNE analysis yielded a very different pattern that featured unevenly distributed and poorly separated cells (FIG. 12C). Moreover, several classic M1/M2 signature genes were rarely expressed in ATMs and/or presented no obvious differences between lean and obese populations (FIG. 19C), despite that the latter was suggested to contain more M1-like subsets, as was identified using other classic M1 markers. These findings and those from more recent studies highlight the inadequacy of the classic model in characterizing macrophage populations. Currently available methods (such as t-SNE and PCA) used to analyze these data focus on similarity comparisons at the whole-transcriptome level, with limited knowledge of details to capture complex biological function, and thus are inefficient at capturing relatively subtle differences within a population, such as multiple activation states of macrophages, and indeed, such limitations of current algorithms have been recently realized and discussed. Thus, to understand the molecular differences between some macrophage cell populations, higher-resolution methods are required.

MPI Characterized the Dynamic Activation Waves of Macrophage Responses In Vitro.

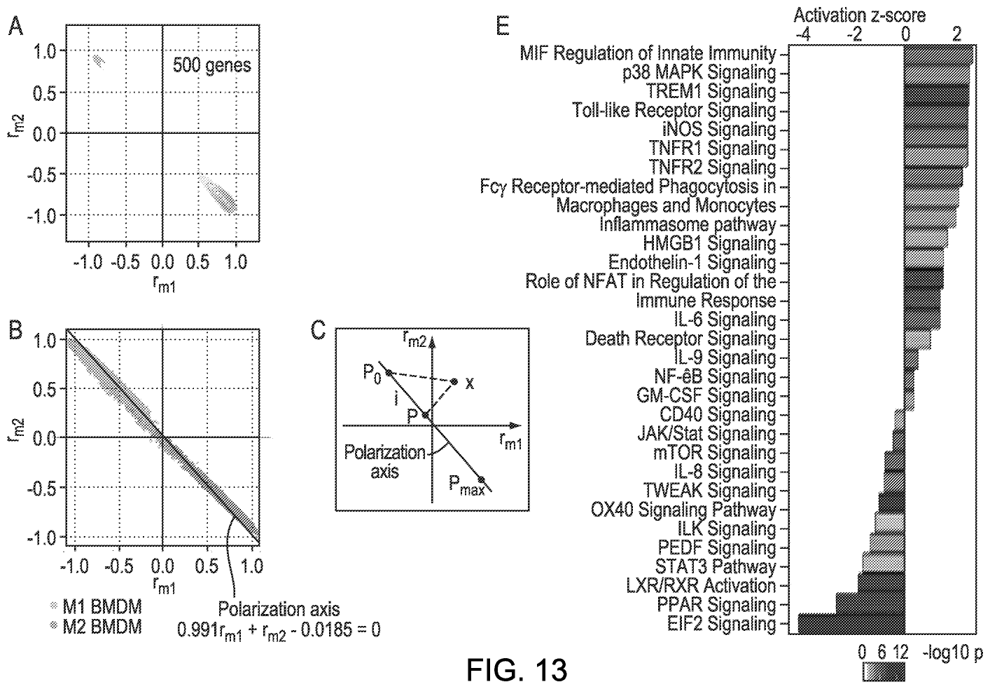

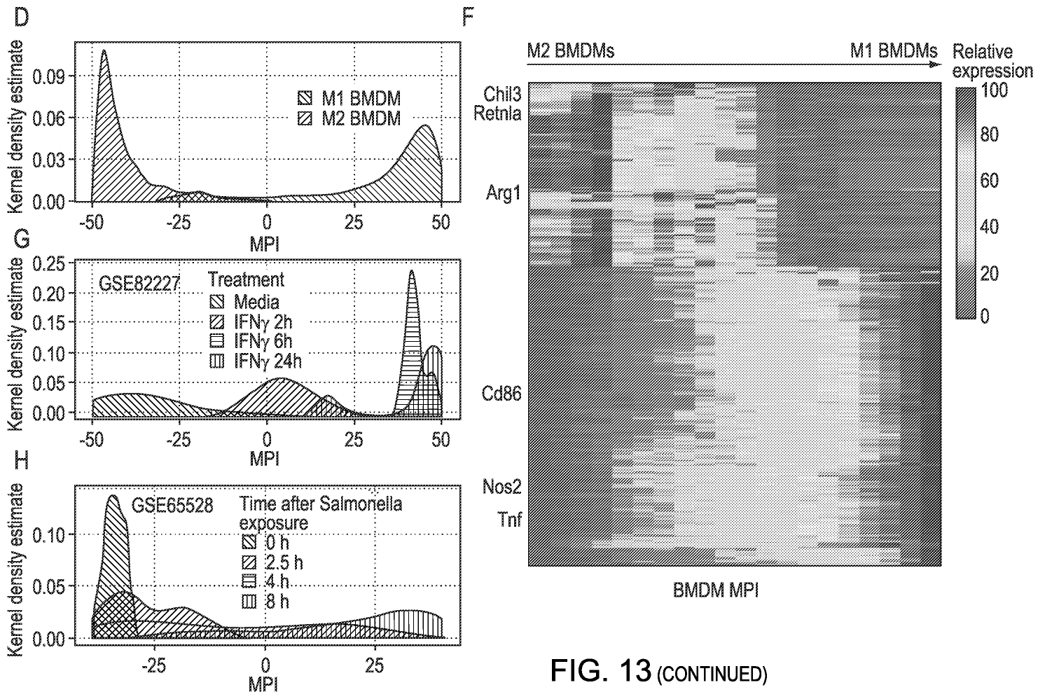

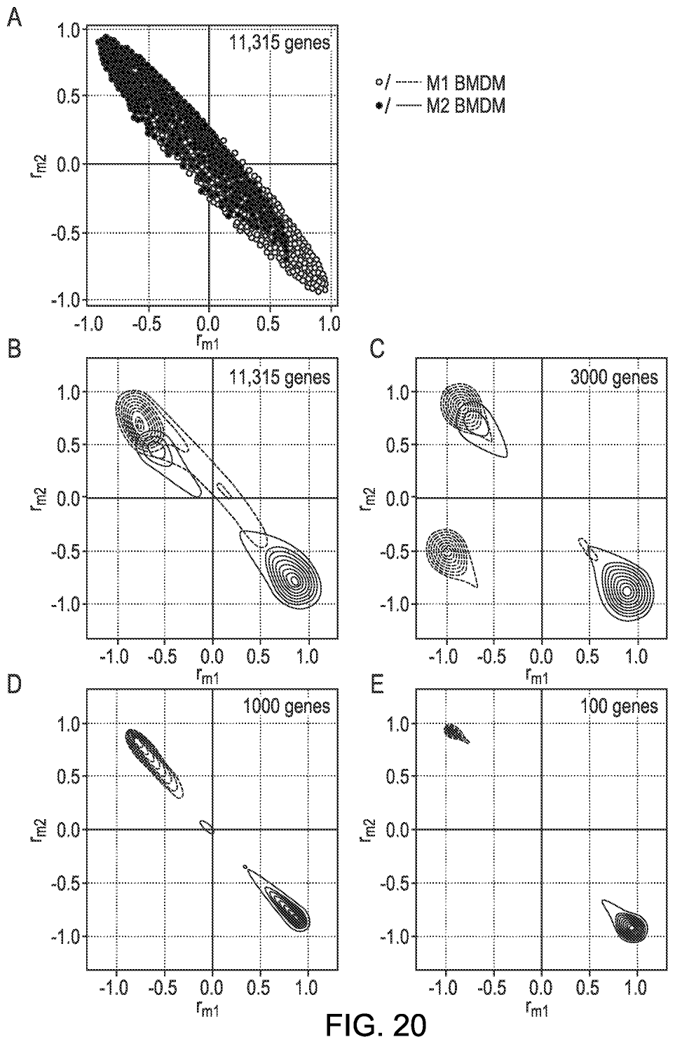

To generate a platform tailored to capture the dynamic yet relatively subtle differences at the whole-transcriptome level among macrophage subpopulations, we took advantage of scRNA-seq profiles from the BMDM polarization system. A series of calculations were performed to identify a gene set that could clearly separate M1 and M2 cells and retained the highest inclusion of the most differentially expressed genes. Groups of top-ranked genes with preferential expression in M1 or M2 samples (FDR-adjusted P value <1×10⁻¹⁰, detectable frequency >1%, unique molecular identifier [UMI]>1) were selected to calculate the similarity of each cell to the average UMI in either M1 or M2 samples using a method modified from Pearson's correlation (FIG. 20). Among all tested groups, the top 500 most differentially expressed genes were selected as the “polarization signature genes” (PSGs) (Table B) because they allowed for efficient separation and yet retained effective gene coverage between M1 and M2 samples (FIG. 13A). Next, a linear regression line was generated of all scRNA-seq profiles plotted by the correlation to M1 or M2 average expression levels of the PSGs (rₘ₁, rₘ₂), which was termed the “polarization axis” (FIGS. 13B and 13C, and Table B). Each cell was then assigned an MPI value with adjusted distance of their projection point to the starting point of the polarization axis (FIG. 13C). M1 and M2 samples were well separated along the polarization axis and classic M1 and M2 markers displayed predicted correlations with MPI values (FIGS. 13D and 13F): higher MPI suggests more M1-like (more inflammatory) states and lower MPI suggests more M2-like (less inflammatory) states. Ontology analysis revealed that the PSGs are enriched for cell signaling pathways that are well recognized as macrophage polarization regulators (FIG. 13E).

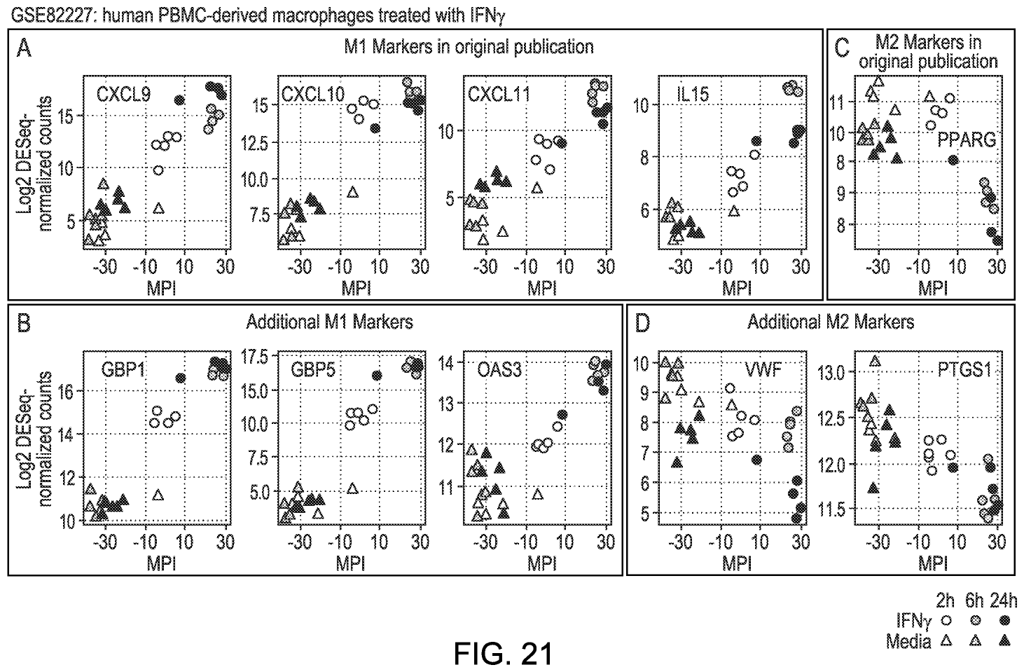

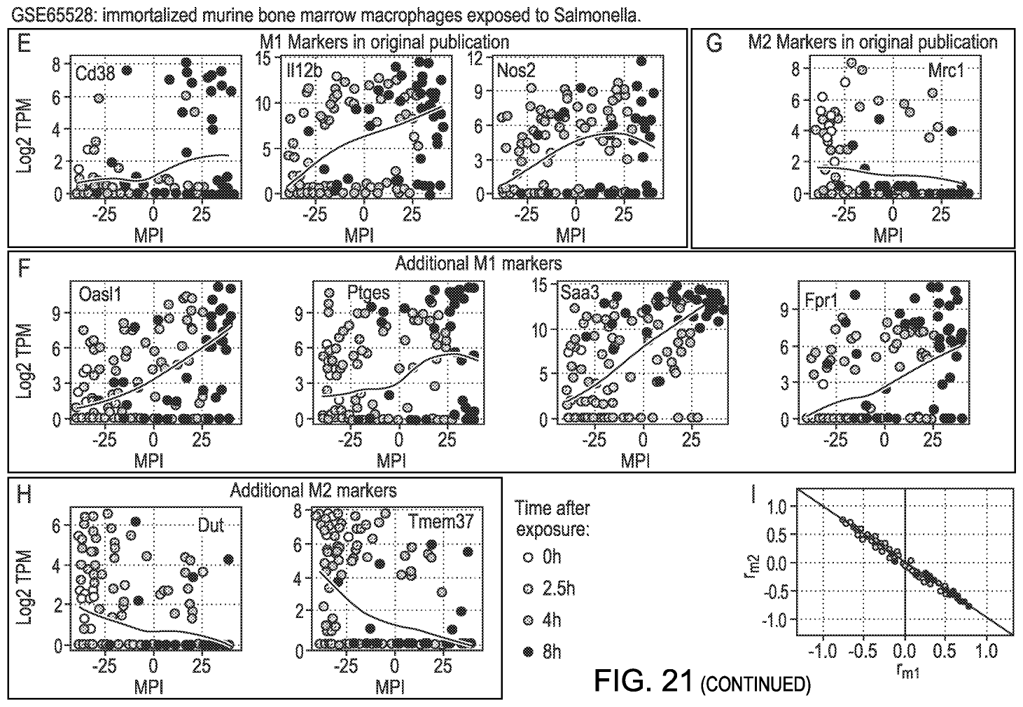

To evaluate the efficacy of MPI in deciphering macrophage activation states, this model was tested with several publicly available macrophage transcriptome data sets from other research groups that included human or murine macrophages challenged with different stimuli (ncbi.nlm.nih.gov/geo/). Interestingly, application of MPI not only demonstrated the activation states of macrophages in these experiments, but also revealed features in a more comprehensive and clearer way compared with previous experiments, such as a clear sequential “activation wave” of human (FIG. 13G) or mouse (FIG. 13H) macrophages from early to late time points after stimulation (FIG. 21). Thus, these validation tests supported the efficacy of MPI to annotate either murine or human macrophage activation states.

AMDI Captures Transition from Preactivated to Activated Macrophages.

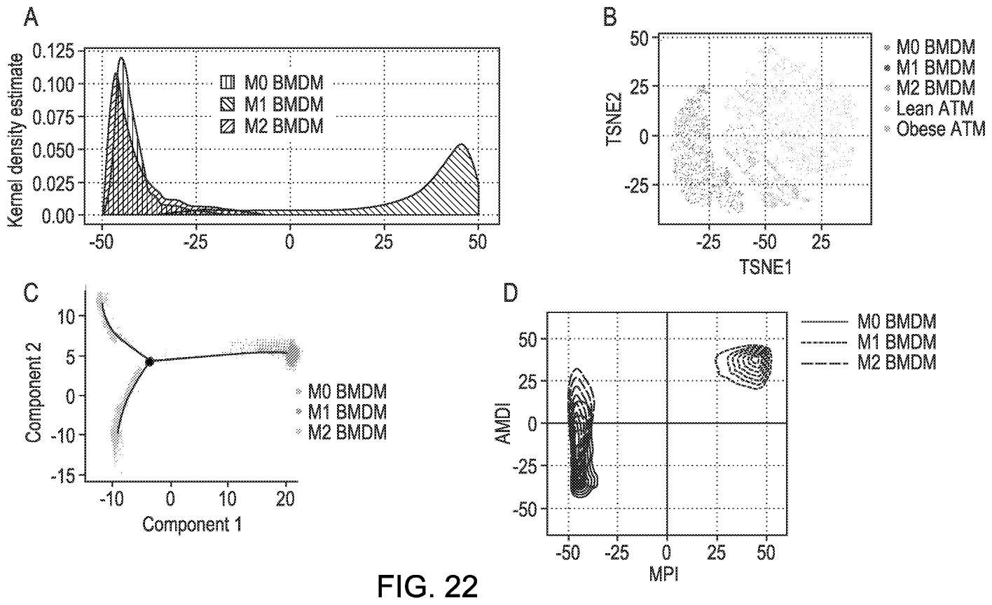

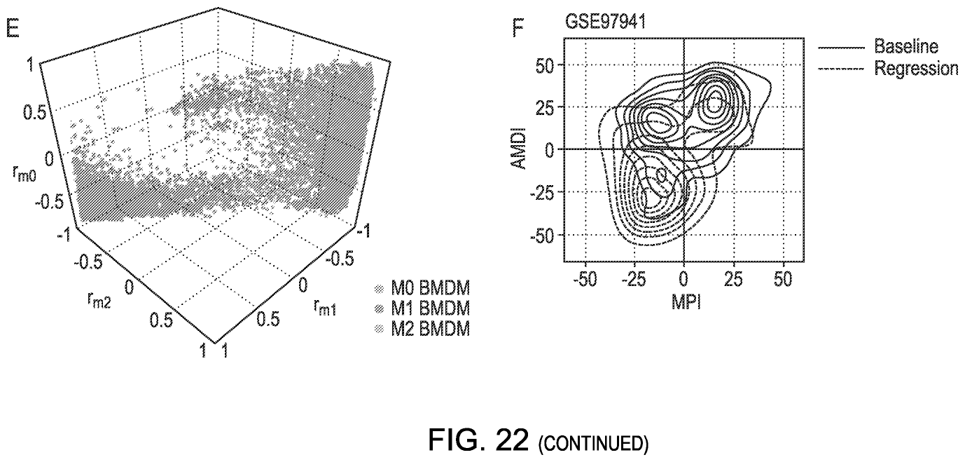

Next, we developed a second index, AMDI, to annotate the process from preactivation states (M0) toward fully activated and matured macrophages (M1/M2). Heterogeneity of tissue macrophages is not only defined by diverse activation states, but also by terminal maturation, i.e., how complete the activation process is. Studies suggest that tissue macrophages are derived from either embryonic or bone marrow precursors. Embryonic-origin tissue-resident macrophages are considered fully mature populations that maintain homeostasis. Under stress conditions, monocytes can also be recruited to tissues and transition into macrophages that either replenish the homeostasis-maintaining tissue-resident population or undergo activation to orchestrate inflammatory responses. Thus, to enable comprehensive investigations of in vivo-derived macrophages under normal or disease conditions, we created an index for macrophage terminal maturity. Indeed, comparison of whole transcriptomes among M0, M1, and M2 BMDMs revealed higher similarity between M0 and M2 than either M0 and M1 or M2 and M1 (FIGS. 13A and 13B). Furthermore, MPI application successfully identified the M1 population but failed to distinguish M0 and M2 (FIG. 22A), suggesting that genes other than PSGs were responsible for the M0 to M2 terminal differentiation process.

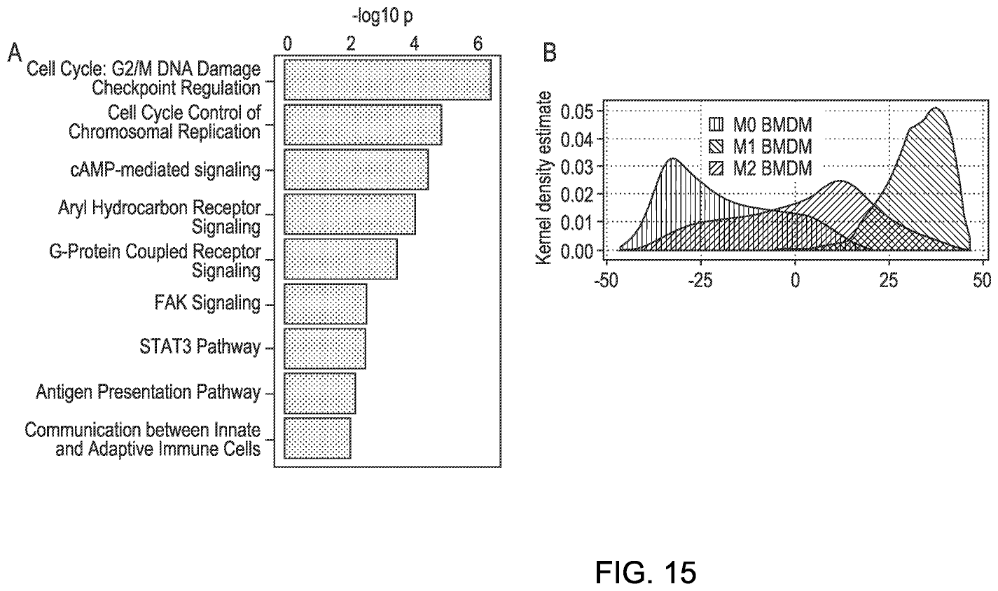

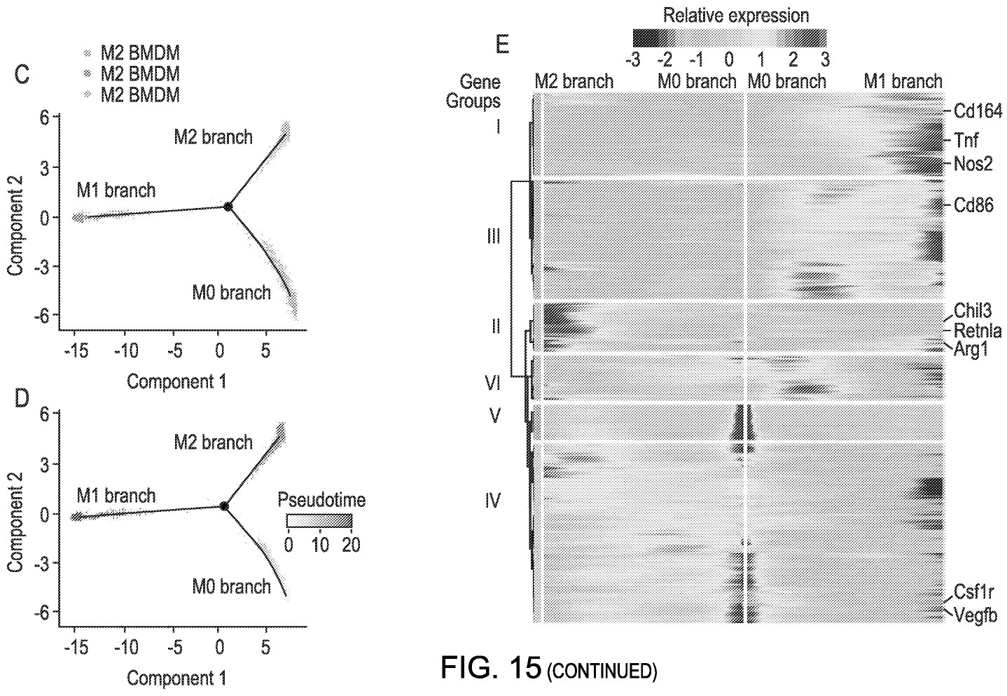

Thus, a list of genes was extracted that correlated with macrophage terminal maturation but not polarized activation, resulting in a total of 435 genes that we defined as “activation-induced macrophage differentiation signature genes” (AMDSGs) (Table B). Ontology analysis confirmed that these genes were highly enriched for factors significantly altered during macrophage terminal maturation (e.g., Csflr, Cebpb, Cd274, Itga4, etc.), canonical signaling pathways for cell cycle control, and development of macrophage functions (antigen presentation, communication between innate and adaptive immune cells, etc.; FIG. 15A). Using the AMDSGs, we calculated the similarities between the averaged M0 BMDM gene expression and each BMDM cell (rₘ₀) and found that cells with higher rₘ₀ indicated higher similarity to M0 BMDMs; this indicated a preactivation and more immature phenotype, whereas cells with negative rₘ₀ (−rₘ₀) suggested less similarity to M0 cells, hence a more mature state. Accordingly, each cell was assigned an AMDI value (see Methods for details) to depict their relative maturity as a macrophage (FIG. 15B). Furthermore, compared with unsupervised clustering with whole transcriptome (FIG. 12D), t-SNE analyses using PSG and AMDSG gene sets presented lean or obese ATMs with cell clusters either overlapping with or close to BMDM clusters, suggesting features that are more comparable to ex vivo culture systems (FIG. 22B).

Importantly, with the application of PSGs and AMDSGs, the 3 BMDM M0/M1/M2 cell populations were assigned to 3 separate branches when cell trajectory algorithms were applied (FIG. 15C); intriguingly, these aligned along a pseudotime progression scale, suggesting a mechanistic process where M0 cells precede M1 and M2 cells (FIG. 15D). PSGs and AMDSGs displayed significantly altered expression patterns within each branch as well (FIG. 15E). In contrast, a cell trajectory plot built using whole transcriptomes presented substantial overlap between M0 and M2 BMDMs (FIG. 22C). In summary, compared with whole-transcriptome-based cell cluster characterization or cell trajectory analyses, application of PSGs/AMDSGs provided a fine-mapping strategy tailored to capture macrophage activation.

MacSpectrum Incorporates MPI and AMDI to Define ATM Subpopulations in Obesity.

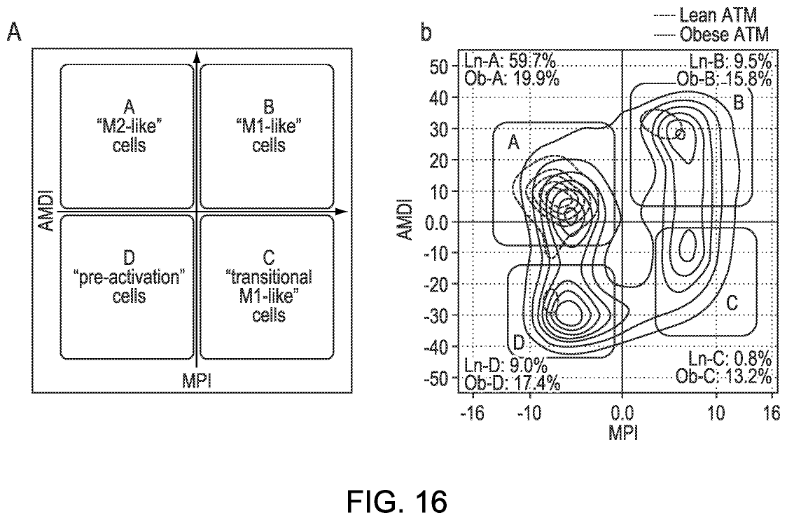

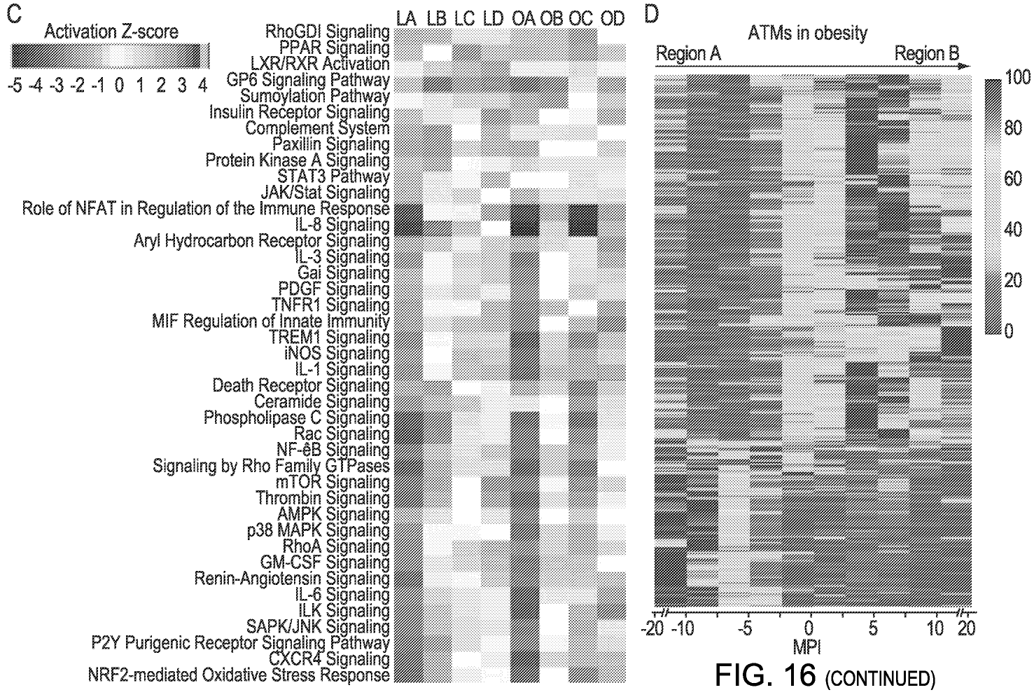

To enable a comprehensive depiction of macrophage activation and terminal maturation states, we incorporated the 2 newly created indices, MPI and AMDI, to establish the MacSpectrum platform (macspectrum.uconn.edu). Based on the combined relative values of MPI and AMDI for macrophages, we designated cells in each region of the MacSpectrum plot as A, “M2-like”; B, “M1-like”; C, “transitional M1-like”; and D, “preactivation” (FIG. 16A). As expected, application of MacSpectrum successfully distinguished all 3 BMDM activation states, M0, M1, and M2 (FIGS. 22D and 22E). We next tested the ability of MacSpectrum to annotate complex in vivo macrophage states from scRNA-seq data sets that recorded artery macrophage responses under atherosclerotic (baseline) or regression conditions (GSE97941). Integrating MPI and AMDI, MacSpectrum effectively depicted a dynamic macrophage subpopulation compositional change from atherosclerotic (baseline) to regression conditions, where a highly matured (high AMDI) and inflamed (high MPI) macrophage pattern transitioned to a more nonactivated and less inflammatory pattern (low AMDI and MPI, region D) (FIG. 22F), which was consistent with the original publication. Thus, these results suggest that the MacSpectrum model developed with expression changes of PSGs and AMDSGs was able to capture characteristic features of macrophages and effectively drive their separation into distinct subpopulations.

Next, MacSpectrum was applied to scRNA-seq profiles of ATMs to dissect their functional contribution to tissue homeostatic maintenance and actions under stress (FIGS. 16B-16D). ATMs are the most abundant immune cells in white visceral fat and play crucial roles in maintaining adipose tissue function and immunological homeostasis. ATMs increase significantly in quantity and proportion in obese visceral stroma, and the whole ATM population has been shown to shift from a less inflammatory M2-like to a proinflammatory M1-like activation state. Given such a distinct phenotypic shift and proportional change between M1-/M2-like states in lean and obese ATMs, single-cell transcriptome analysis would be expected to display clear clusters of M1 or M2 cells. In practice, however, lean and obese ATMs analyzed using the t-SNE algorithm overlapped with themselves in a cluster that was separate from polarized M1 or M2 BMDM clusters (FIG. 12D). Of note, classic M1/M2 signature genes were rarely or weakly expressed in ATMs and/or presented no obvious enrichment in either lean or obese populations (FIG. 19C), despite a report that obese ATMs contain more M1-like subsets.

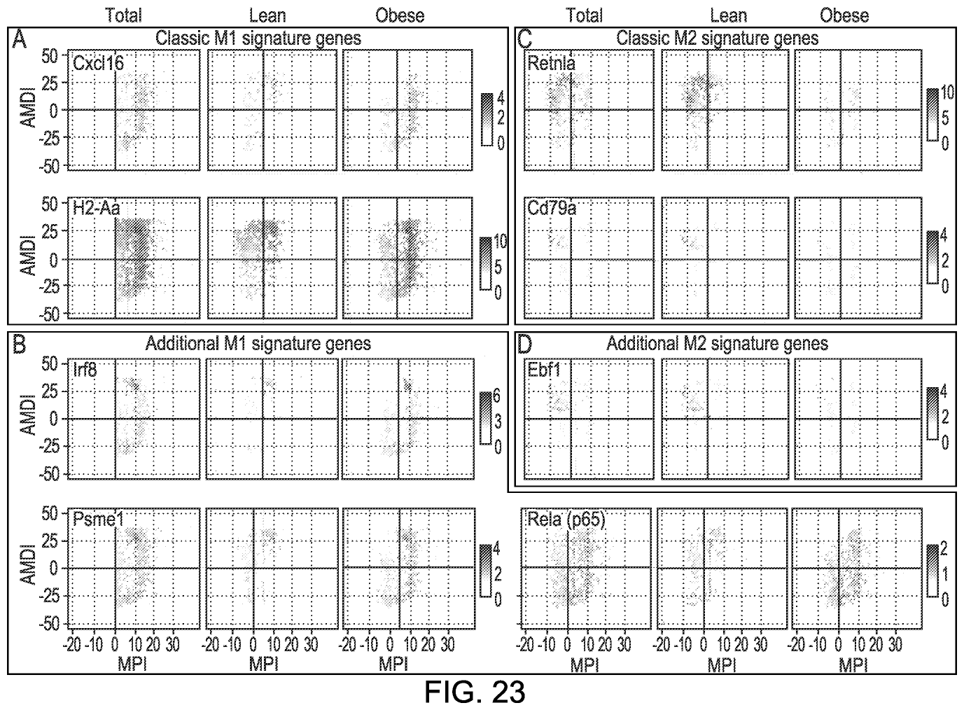

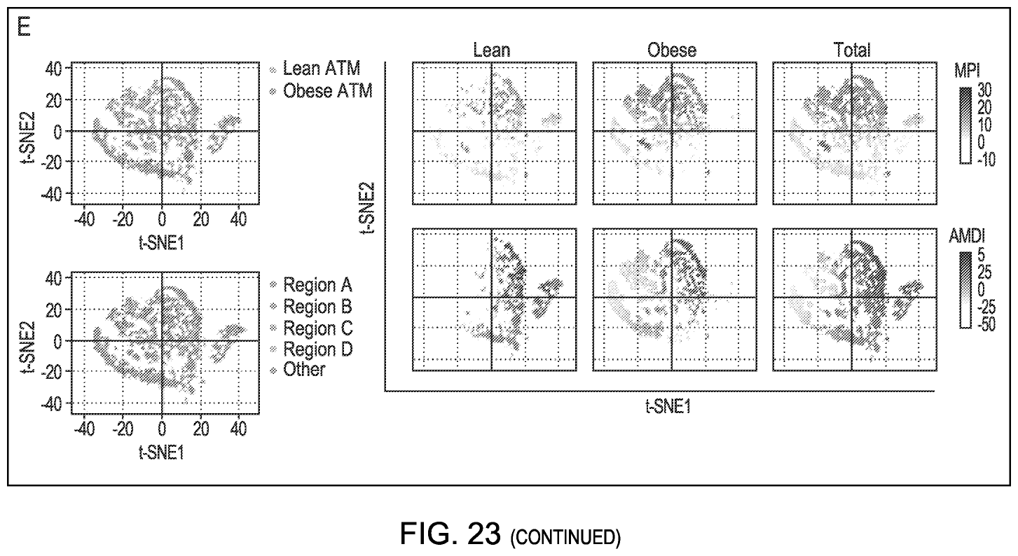

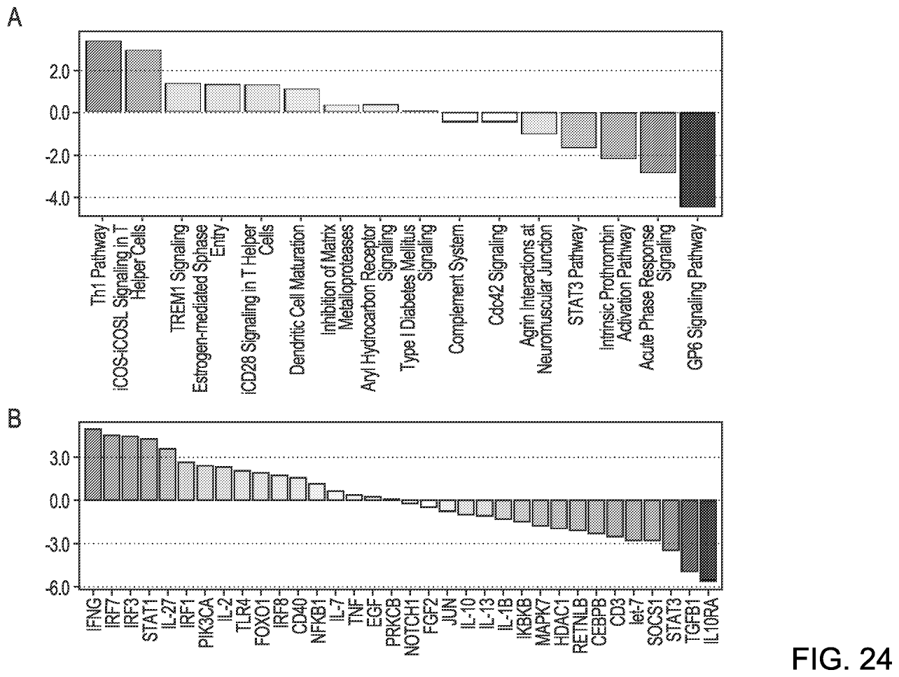

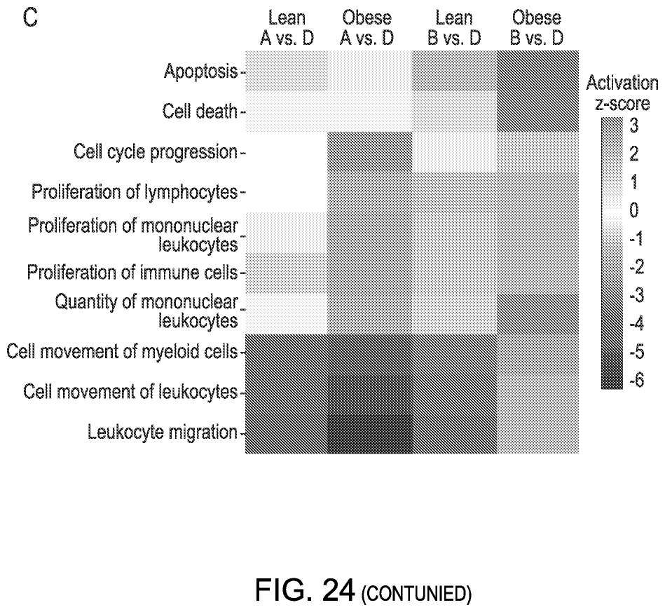

MacSpectrum successfully identified various ATM populations with distinct activation and maturation states (FIG. 16B and FIGS. 23A-23D). Specifically, ATMs from lean tissue were predominantly enriched in region A, defined by cells with high AMDI and low MPI, while ATMs from obese tissue displayed a diversified profile with cells clustered in all 4 regions (FIG. 16B). Compared with ATMs from lean mice, ATMs from obese mice had an overall increased proportion of cells with high MPI, and thus a greater proinflammatory activation status.

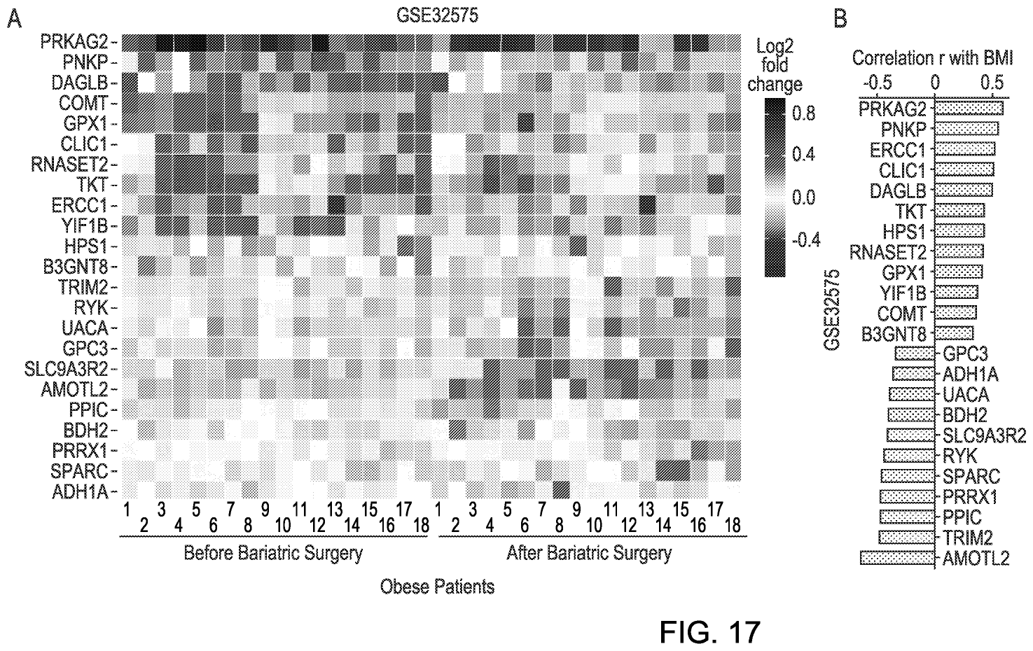

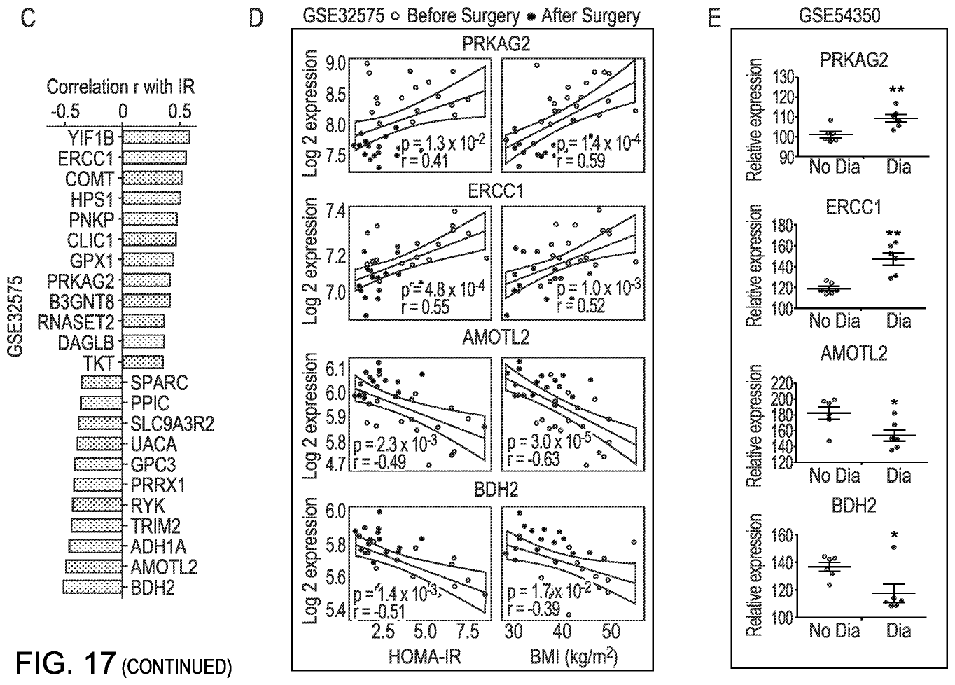

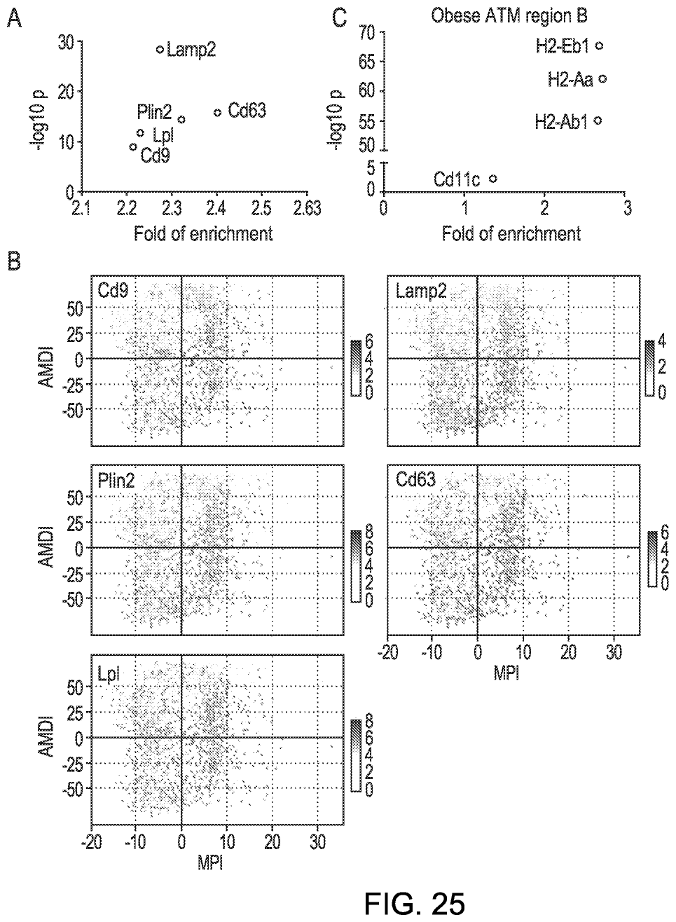

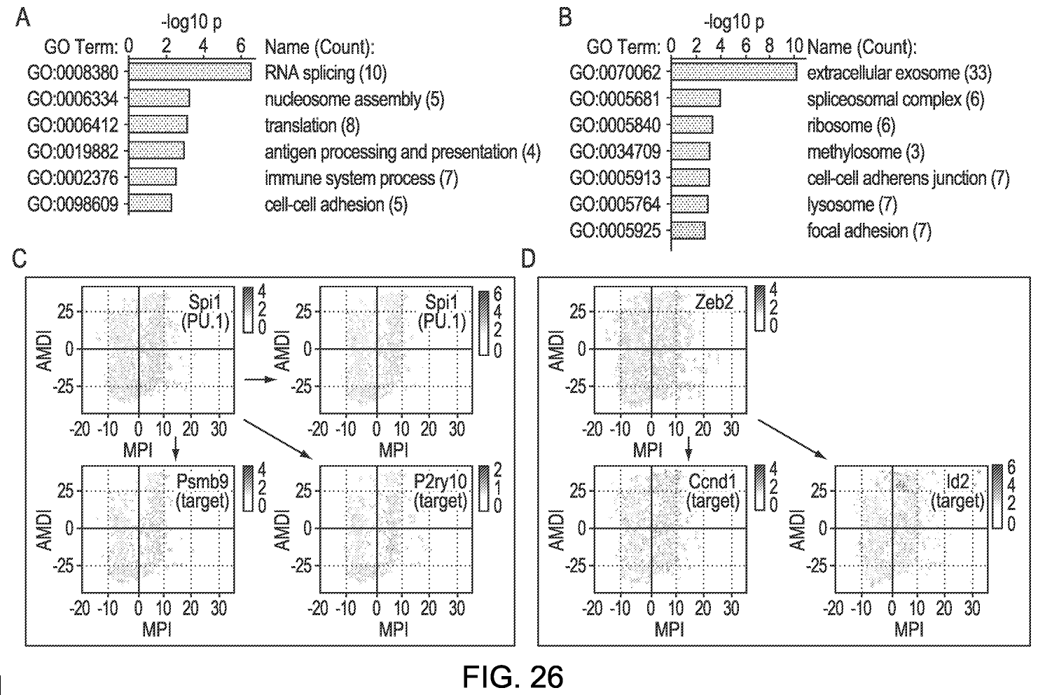

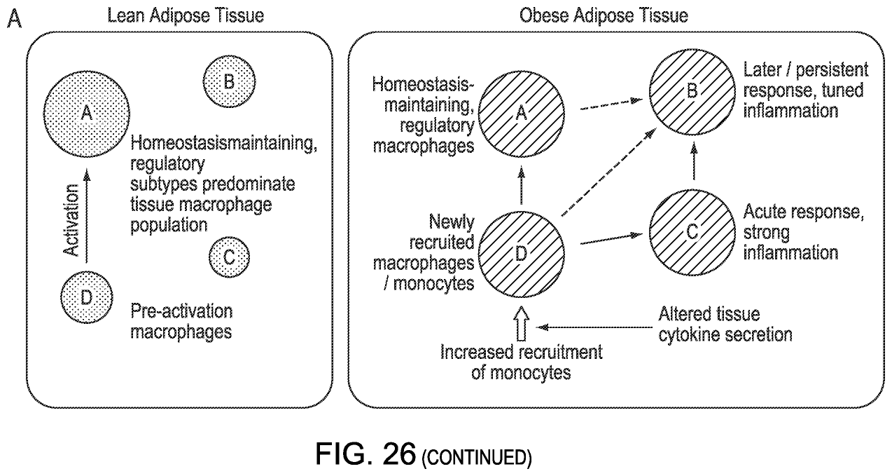

To precisely characterize ATM subpopulation properties and functions, we performed gene ontology and pathway analyses on cells from each region (A-D) (FIG. 16 and Supplemental FIG. 17). Overall, obese-tissue ATMs presented enhanced proinflammatory profiles compared with ATMs from lean tissue and contained a significantly higher proportion of cells in regions B and C (high MPI) (FIG. 16B). Of note, cells in region B (high MPI/high AMDI) primarily consisted of obese ATMs with mature and strong inflammatory properties (FIG. 16C), while the obese-dominated region C showed similar gene signatures (FIGS. 25A-25B) with a previously reported proinflammatory CD9⁺ ATM subpopulation found in obese mice, consistent with its high MPI values. On the other side of the spectrum, region D cells exhibited early maturity (lowest AMDI) and low inflammatory features (low MPI) among all the ATM subpopulations, which might represent more monocytic cells that are newly recruited from peripheral blood. However, their origin requires further study, especially for lean ATMs which are suggested to primarily derive from embryonic origins. Interestingly, the macrophage/dendritic cell differentiation regulators Spi1 (PU.1) and Zeb2 and their downstream target genes were enriched in regions C and B of the obese ATMs, respectively (FIGS. 26C-26D). Moreover, previously reported dendritic cell signature genes including Cd11c and several MHC class members were enriched in region B of the obese ATMs (FIG. 25C), indicating potential contribution of dendritic cells to obesity-induced adipose tissue inflammation. However, considering the often overlapping characteristics of dendritic cells and macrophages, the specific identity of those cells requires further investigation.

Based on these observations combined with gene ontology studies (FIGS. 26A-26B), we propose a model of obesity-induced alterations in ATM subpopulations (FIG. 26E). In lean adipose tissue, preactivation and less mature macrophages (D) primarily develop into resident M2 cells (A) to carry out tissue maintenance functions. In obese adipose tissue, metabolic and immunological cues in the white visceral adipose tissue enhance monocyte recruitment, activate a portion of cells into resident M2 ATMs (A), and activate another portion of cells into an acute/strong (C) then later a tuned/moderate inflammation state (B) (FIG. 26E), resulting in obesity-induced adipose tissue inflammation.

MacSpectrum Identifies Unique Gene Sets in Diabetic Conditions in Obesity.

Plasticity is a key feature of macrophages that allows activation of condition-specific functions through specific signaling pathways in response to diverse stimuli. The proportions of cells across the 4 regions of MacSpectrum (FIG. 16A) dictate the overall macrophage population character under a given condition, and can be used to identify stimulation- or disease-specific signature genes. Taking advantage of MacSpectrum resolution for defining complex macrophage subpopulations in vivo, we performed several comparisons to identify genes that could represent early biomarkers for obesity-related conditions in humans. We first identified genes with preferential expression in (a) “preactivation” cells, by comparing obese versus lean ATMs in region D from mice; and (b) mature macrophages with polarized inflammatory states, by comparing obese ATMs in region B with lean ATMs in region A from mice. We then cross-compared these gene sets to identify signature genes expected to be involved in the early stages of obesity inflammation. Finally, to determine whether any of these genes correlated with human conditions related to obesity, we cross-compared these genes with those from (i) obese human patients with and without diabetes, and (ii) obese human patients before and after bariatric surgery.

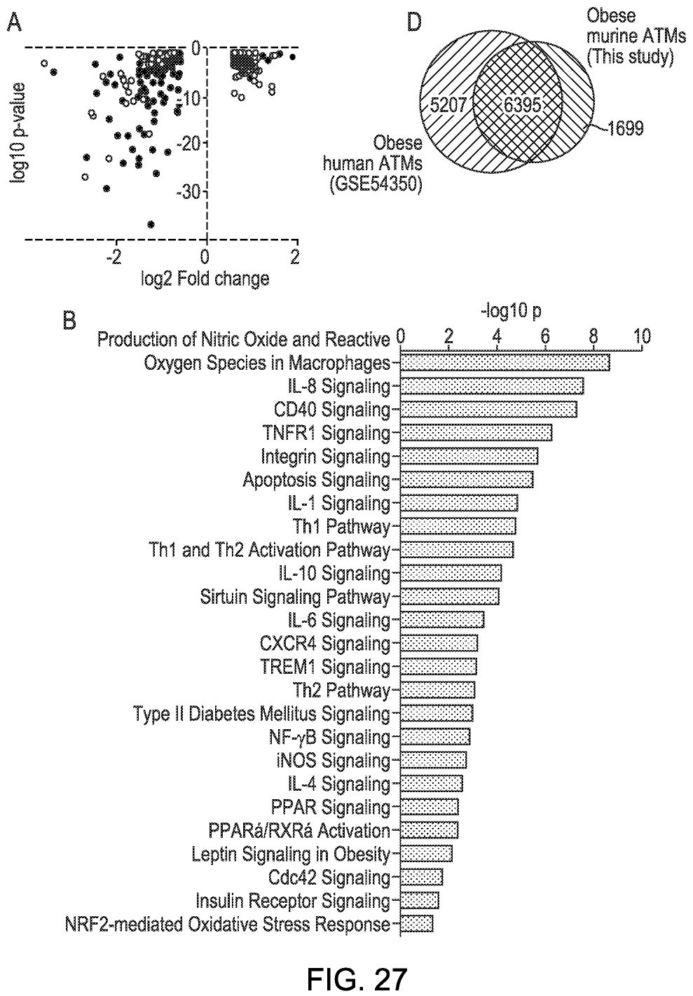

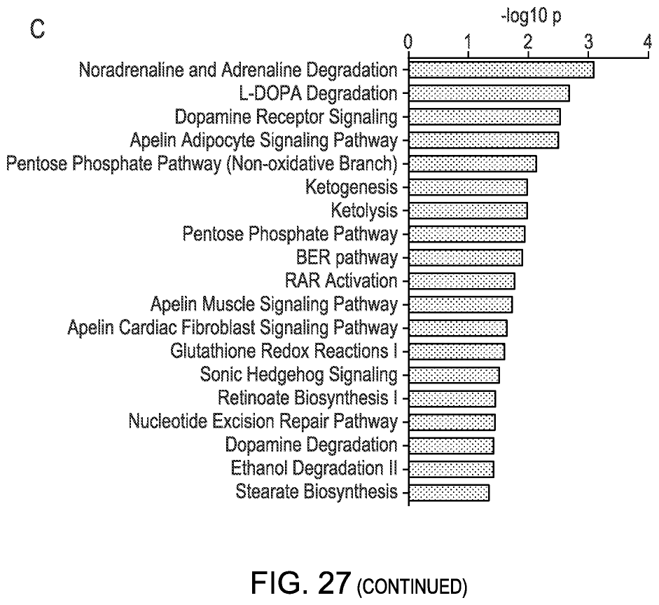

As shown in FIG. 27A, a total of 603 mouse genes displayed significant differential expression in obese versus lean preactivation (region D) cells (P<0.05, fold change >1.5). Among them, over 60% of genes consistently displayed polarization-associated expression preference (385 out of 603 genes, P<0.05, fold change >1.5 comparing obese M1-like cells in region B vs. lean M2-like cells in region A). Interestingly, 436 genes expressing a distinct macrophage polarization pattern in murine ATMs also displayed significant differential expression (P<0.05) in CD14+ cells isolated from obese human subjects with or without diabetes (GSE54350). Signaling pathway analysis of these genes revealed enriched TNFR, IL-8, and IL-1 inflammatory signaling pathways and those involved in oxidative stress response (FIG. 27B).

To evaluate the contribution of monocytes in the pathogenesis of obesity-induced T2DM, we selected preactivation cells in region D mapped by MacSpectrum for comparison to circulating monocyte profiles generated from obese patients who underwent bariatric surgery (an effective procedure to ameliorate obesity-induced insulin resistance and metabolic disorder risks). A total of 185 genes that displayed differential expression patterns in murine preactivation ATMs (obese vs. lean ATMs in region D) were also significantly altered (P<0.05) in circulating monocytes isolated from 18 patients before or 3 months after surgery. To identify genes that are important for ATM polarization in murine obesity but also altered in human obese subjects with or without diabetes (GSE54350) or associated with improved metabolic stress (GSE32575), we cross-examined the lists of genes with significant expression differences (P<0.05) in each comparison and identified a total of 23 gene candidates (FIG. 17A). Gene ontology analyses of these genes indicated roles in both inflammatory and metabolic regulation of monocytes/macrophages (FIG. 27C). Among this set of genes, several were previously reported as diabetes-associated genes by different studies, such as Erec1, Comt, Clic1, Gpx1, Rnaset2, Tkt, Sparc, Gpc3, Prrx1, and Bdh2. In addition to these known diabetes-related genes, we also identified 13 genes that potentially contribute to T2DM development in obese populations. For example, PRKAG2 encodes a member of the noncatalytic subunit of AMP-activated protein kinase (AMPK) gamma unit family that mediates binding to AMP/ADP/ATP for activating/inhibiting AMPK function. The function of PRKAG2 has not been analyzed in T2DM; however, mutations of this gene may lead to Wolff-Parkinson-White syndrome and other glycogen storage diseases in heart.

In the mega-analysis using MacSpectrum, we found that PRKAG2 displayed significant correlation with BMI (P<0.05, r=0.59) and homeostasis model assessment of insulin resistance (HOMA-IR) (P<0.05, r=0.41) in obese patients that underwent bariatric surgery (FIGS. 17A-17D), which concomitantly displayed an expression pattern preferentially in CD14⁺ ATMs from obese subjects with diabetes than those without diabetes (FIG. 17E). AMOTL2, a protein encoded by another gene also less known to contribute to T2DM pathogenesis, can bind to angiostatin to modulate angiogenesis and cell-matrix remodeling through inhibition of the Wnt/β-catenin pathway. AMOTL2 was suppressed in obese diabetic subjects compared with nondiabetic subjects (FIG. 17E); concomitantly, the expression pattern of AMOTL2 was negatively correlated with BMI (P<0.05, r=−0.64) and HOMA-IR (P<0.05, r=−0.49; FIGS. 17A-17D). Expression patterns in obese subjects with or without diabetes, the expression distribution with Pearson's correlation values to BMI or HOMA-IR, and the functions involved in metabolic/inflammatory regulation of each gene in this 23-gene set. Taken together, the results identified a signature of markers that distinguish T2DM risk in obese subjects in circulating monocytes, suggesting their potential functional contribution to the pathogenesis of T2DM in obesity.

Discussion

Macrophages are often the most dominant tissue immune cells that contribute to both tissue homeostasis and diseases. Under normal conditions, macrophages primarily maintain a noninflammatory state by facilitating tissue regeneration and wound healing. In response to immune challenges, macrophages are essential to innate immunity, with a diversified and quick response to invading pathogens and an ability to clear autologous tissue waste. Macrophages in various tissues may have either embryonic or bone marrow-derived hematopoietic origins, but their function in maintaining tissue homeostasis or responding to acute or chronic stress may be interchangeable. Given the important function of macrophages and their direct link to circulating monocyte recruitment under stress conditions, researchers have invested great efforts to delineate their diversified phenotypes and swift responses to both systemic and tissue microenvironmental cues. However, while functional annotation of macrophages in vivo is both necessary and heavily pursued, the precise landscape of macrophages in tissues under both healthy or stress conditions remains vague.

Technologies such as scRNA-seq have the potential to advance our understanding of macrophages in vivo by facilitating investigations into macrophage maturation and activation states at the individual cell level. However, current algorithms for mining such data rely on similarities and differences at the overall transcriptome level. Compared with features that define cell lineages, signals altered during macrophage terminal maturation and activation are relatively less distinct, as we observed in this study and others have pointed out as one of the major computational challenges in scRNA-seq clustering. To address this technical challenge, we generated a platform, MacSpectrum, specifically designed to capture macrophage dynamic changes during tissue infiltration and activation by incorporating original algorithms and scRNA-seq profiles from multiple macrophage data sets. Based on 2 created indices, MPI and AMDI, that fine-map each macrophage with a combination of terminal maturity and polarization states, MacSpectrum enables effective macrophage functional annotation with high resolution and sensitivity. The capability of MacSpectrum to segregate macrophage heterogeneity was well supported by its performance on samples from human and murine species, under in vitro and in vivo conditions, and in bulk and single-cell sequencing formats. The spectrum-like model of macrophage activation states revealed in the present study is consistent with previous, and MacSpectrum further provides quantitative indices that more finely map each macrophage, and thus will enable researchers to define macrophage subpopulations with high resolution that encompass inflammatory/antiinflammatory states as well as intermediate subpopulations from complex in vivo microenvironments under normal or stressed/diseased conditions. It should be noted that there are other features of macrophages, such as origins (e.g., bone marrow- vs. yolk sac-derived), that cannot be deciphered by MacSpectrum currently. In addition, the performance of MacSpectrum with human data sets will need to be examined more thoroughly to address several challenges, which include limited human monocyte/macrophage data availability, procedural variations, small sample sizes, and large variations in individual medical backgrounds.

Although MacSpectrum was built on murine macrophage (BMDM) gene expression profiles, the application strategy we developed does not rely on similarity comparisons between samples from one study (such as murine BMDMs, our starting point) to those from another study (any other mouse or human monocyte/macrophage study). The MacSpectrum strategy involves using signature gene lists, novel algorithms, and study-specific data to build indices. Two comprehensive signature gene lists (PSGs and AMSDGs; >400 genes each) were generated that are most relevant to macrophage action based on a strong BMDM polarization model. Then, when MacSpectrum is applied to analyze any other macrophage/monocyte study, gene IDs from the PSGs and AMSDGs are extracted from the study data to be analyzed and their expression levels are used to calculate the MPI and AMDI indices, respectively. Thus, values of MPI and AMDI for each sample are only relative within the same study to capture the unique activation features for each study context. To test the efficacy of MacSpectrum, its ability to annotate human macrophages under various physiological and pathogenic conditions was rigorously tested. Multiple sets of transcriptome profiles from human monocytes/macrophages that were isolated from different physiological conditions or exposed to various pathogens (GSE82227, GSE32575, GSE54350, GSE36952, and GSE92495) were compared. It was found that close to 80% of expressed genes were shared by murine or human macrophages, consistent with previously reported conservation between monocytes/macrophages of the 2 species. More importantly, these genes also displayed comparable expression patterns during activation (FIG. 13, FIG. 17, FIG. 21, and FIG. 27). Of note, these data were also generated using different platforms, such as microarray, conventional RNA-seq, or scRNA-seq, suggesting MacSpectrum exhibits tolerance for analyzing transcriptome profiles across techniques with varying depths. Thus, MacSpectrum displayed high potential to characterize monocyte/macrophage actions in both human and mouse models under normal or disease conditions. At its current stage, due to limitations of profiling technology, MacSpectrum aims to provide fine-mapping information of monocytes/macrophages within each study, rather than cross-study comparisons. It is acknowledged that this model can be further strengthened and applied to human diseases when more unified standards in the profiling technology and more monocyte/macrophage data sets are available.

In addition to consistent identification of known features, MacSpectrum demonstrated great potential for uncovering novel characteristics of macrophages in tissues, as well as stimulation-specific gene sets. Under conditions of acute or chronic stress, circulating monocytes are recruited into tissues and subsequently undergo terminal differentiation and activation upon exposure to microenvironmental cues. Chronic inflammation is a hallmark of obesity and a risk factor for obesity-induced T2DM; this is accompanied by systemic metabolic and inflammatory exposure to circulating monocytes before their recruitment to obese adipose tissue, which may contribute to further differentiation and activation of the process locally. Therefore, it is not surprising to identify genes sharing similar expression preference in ATMs and circulating monocytes that allow for differential diabetic states in obese subjects.

Indeed, several studies have defined links between obesity, inflammation, circulating monocytes, and differential gene expression. Obesity can induce changes in miR expression in circulating monocytes; miR-146b-5p, which decreased in monocytes during obesity, is a major mediator of the antiinflammatory action of globular adiponectin. A separate study found an association between COX4I1 depression, insulin resistance, and T2DM in obesity, and its expression in monocytes reflected that in adipose tissues. In addition, researchers observed equal changes in abdominal adipose tissues from obese diabetic humans and mice.

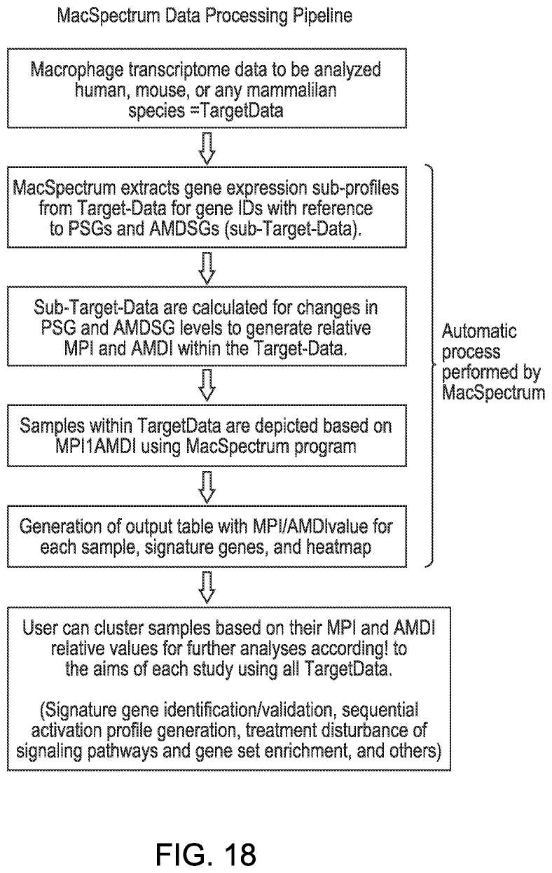

These results support MacSpectrum as a framework to readily annotate previous macrophage classifications (e.g., M1 vs. M2) and as a systematic approach for dissecting the “many alternative faces of macrophage activation.” Clear separation of diverse macrophage populations along with functional annotation and identification of underlying tissue- or disease-specific gene signatures, as we initiated here for diabetes, will facilitate more focused therapeutic development. The data processing pipe of MacSpectrum is summarized in FIG. 18.

MacSpectrum enabled fine-mapping of individual cells with respect to their terminal maturity and polarization states, and therefore allowed the comprehensive analysis of tissue macrophages. The shift of each macrophage subpopulation along the MPI and AMDI axes also facilitated a more precise annotation of the overall macrophage subpopulation dynamic under various physiological conditions. Of note, macrophage transcriptional activation programs are loyal to the outcomes imposed by specific stimuli, which are often multifactorial and with various magnitudes. Therefore, it was expected to observe tissue- or disease-specific features when analyzing macrophages. Indeed, top-ranked genes displayed MPI and AMDI correlations while sharing less than 15% overall commonality between macrophages from visceral adipose tissue in diet-induced obesity and aorta macrophages isolated from an atherosclerosis model (data not shown). Therefore, stimulation-specific gene sets should be studied with different disease and tissue contexts, which is planned to further optimize the MacSpectrum system. This will facilitate the enhanced characterization of macrophages as well as other heterogeneous cell populations.

Methods

Patients and Ethics Statement

Human data collected in a previous study were provided for our mega-analyses in this study. In summary, the original study complies with the Declaration of Helsinki and the Medical Ethics Committee of the Katholieke Universiteit Leuven approved the study protocol. As stated in the previous study, all human participants gave written informed consent. The cohort comprised 18 obese individuals without clinical symptoms of cardiovascular disease (BMI: 45.1±1.4 kg/m², P<0.001 compared with lean controls). These 18 morbidly obese subjects were referred to the hospital for bariatric surgery. Before they were included, individuals were evaluated by an endocrinologist, an abdominal surgeon, a psychologist, and a dietician. Only after multidisciplinary deliberation did the selected patients receive a laparoscopic Roux-en-Y gastric bypass (bariatric surgery). CD14⁺ monocytes were collected before and 3 months after bariatric surgery (BMI: 37.5±1.3 kg/m², P<0.001 compared with before weight loss), total RNA was extracted from these cells, and genome-wide expression analysis was performed. Insulin resistance was calculated as follows: HOMA=fasting plasma insulin (mU/l)×fasting blood glucose (mM)/22.5. The samples were collected between Mar. 29, 2005 and May 30, 2006.

Animals

Mice (C57BL/6J) were purchased from The Jackson Laboratory (stock no. 000664), housed in a 12-hour light/12-hour dark cycle, and provided ad libitum access to food and water for the duration of the study unless stated otherwise. Male mice 5-6 weeks of age were used for feeding analyses. For dietary feeding studies, mice were fed with a high-fat diet (HFD; 60% fat calories, 20% protein calories, and 20% carbohydrate calories; catalog D12492, Research Diets, Inc.) or Teklad standard chow diet (18% fat calories, 24% protein calories, and 58% carbohydrate calories; catalog 2918, Envigo) for 12 weeks. After the feeding regimen, mice were subjected to phenotype characterization, metabolic assays, and postmortem tissue collection for cell isolation.

BMDM Differentiation and Polarization

BMDMs were prepared as previously described. In brief, bone marrow cells were collected from the femur and tibia bones followed by erythrocyte lysis with ammonium chloride solution (8.3 g/l ammonium chloride, 1.0 g/l potassium bicarbonate, and 0.009% EDTA) and seeded in tissue culture plates at a concentration of 0.15×10/cm². Cells were induced for differentiation to monocytes in BMDM growth medium (Iscove's Modified Dulbecco's Medium [IMDM]+10% FBS+15% L-929 cell [ATCC, CCL-1]) for 7 days; fresh BMDM growth medium was replaced on day 3. Maturation and purity of BMDMs were evaluated on day 7 using flow cytometry analysis with fluorescence-conjugated antibodies against CD11b and F4/80. To induce polarization, BMDMs were stimulated 48 hours with 100 ng/ml LPS and 50 ng/ml IFN-γ for M1 activation, or 20 ng/ml IL-4 and 20 ng/ml IL-13 for M2 activation. Unstimulated BMDMs were collected as M0 state.

Isolation of Stromal Cells from Visceral Adipose Tissue

Visceral adipose tissues (epididymal, retroperitoneal, and mesenteric fat pads) from 5 lean and 5 obese C57BL/6J male mice were isolated separately, mechanically dissected, and digested in Hank's balanced salt solution (HBSS) containing 20 mM HEPES, 0.015 g/ml bovine serum albumin (BSA), and 2 mg/ml collagenase II (Invitrogen) for 1 hour at 37° C. in an InGeneron ARC tissue processing system with periodic rotating and inverting. After removing erythrocytes with ammonium chloride solution, cells were filtered through a nylon mesh bag (250-300 μm, catalog 50-303-41, Thermo Fisher Scientific), then a 100-μm cell strainer. Cells were resuspended in PBS with 2% FBS, transferred into a BSA-coated tube, and left on ice before sorting.

FACs

Unless otherwise specified, antibodies (Abs) were from eBioscience. VSCs, ATMs, and BMDMs were stained with fluorescence-tagged Abs for lineage examination. Macrophages were detected with Abs against CD45.2 (catalog 17-0454-82), F4/80 (catalog 25-4801-82), CD11b (catalog 12-0112-82), CD206 (catalog 141706; BioLegend), and CD11c (catalog 12-0114-83). Macrophage activation was measured with Abs against CD206, CD11c, CD80 (catalog 12-0801-85), CD69 (catalog 45-0691-82), and CD86 (catalog 17-0862-82). ATMs were defined as CD45.2⁺CD11b⁺F4/80⁺ cell populations and sorted on a BD Biosciences FACSAria II cell sorter. Approximately 60,000 ATMs were isolated from lean and obese samples and used for scRNA-seq analysis.

scRNA-Seq Library Preparation and Data Processing

BMDM cells from M0, M1, and M2 populations were analyzed separately for scRNA-seq and MacSpectrum development. ATM cells pooled from 5 lean and 5 obese C57BL/6J male mice were pooled prior to scRNA-seq. All cells were resuspended in DPBS with 0.04% BSA, and immediately processed for scRNA-seq as follows. Cell count and viability were determined using trypan blue on a Countess FL II, and approximately 12,000 cells were loaded for capture onto the Chromium System using the v2 single cell reagent kit according to the manufacturer's protocol (10× Genomics). Following capture and lysis, cDNA was synthesized and amplified (12 cycles) as per manufacturer's protocol (10× Genomics). The amplified cDNA from each channel of the Chromium System was used to construct an Illumina sequencing library and was sequenced on a HiSeq 4000 with 150-cycle sequencing (asymmetric reads per 10× Genomics). Illumina basecall files (*.bcl) were converted to FASTQs using CellRanger v1.3, which uses bcl2fastq v2.17.1.14. FASTQ files were then aligned to mm10 mouse reference genome and transcriptome using the CellRanger v1.3 software pipeline with default parameters as reported previously; this demultiplexes the samples and generates a gene versus cell expression matrix based on the barcodes and assigns UMIs that enables determination of the individual cell from which the RNA molecule originated. Gene expression was normalized using CellView software. Briefly, the number of gene transcripts per cell was multiplied by the median of transcripts across all the cells, and then log 2 transformed (following an addition of +1 pseudocount to prevent log error where the transcript count is 0; i.e., log 2[0+1]=0), resulting in normalized expression (NE) values. For clustering, genes were selected based on normalized dispersion analysis.

Dimensionality reduction was performed using CellRanger and CellView pipeline with the 1000 most over-dispersed (i.e., variance/mean NE) genes using Barnes-Hut t-SNE with default parameters, and cell clusters were determined using DBSCAN (eps=5.0, minpts=15). Clusters were visualized (FIGS. 12B-12B, and FIG. 19B) using the t-SNE 2D graph.

Selection of PSGs and AMDSGs

A total of 6979 M0, 4736 M1, and 6391 M2 BMDM scRNA-seq transcriptome profiles were generated. An equal number (4736 cells) of scRNA-seq profiles were randomly selected from M0, M1, or M2 samples and used for further analysis.

PSGs

To select genes with the most significant difference during M1 and M2 polarization, we first filtered all selected scRNA-seq sets for genes with UMI greater than 1 in at least 1% of cells in either M1 or M2 samples, resulting in a total of 11,315 unique gene IDs. Differentially expressed genes between M1 and M2 samples were calculated as log 2 (fold change) and tested using the FDR-adjusted P value of Welch's t test. A total of 6267 genes were selected with an FDR-adjusted P value less than 1×10⁻¹⁰. These genes were then ranked high to low according to their absolute log 2 (fold change) values. As shown in FIG. 12A and FIG. 20, top-ranked genes with the most significant differences were selected to test efficacy of separating M1 or M2 samples. The top 500 most significantly changed genes with P values less than 1×10⁻¹⁰ (FDR) were selected for further application, and termed the PSGs.

AMDSGs.

To identify genes that were significantly altered after M0 exposure to stimuli in M1/M2 activation, we performed the following steps: gene expression profiles of M1, M2, or M0 consisted of a total of 11,566 genes that were expressed (UMI>1) in more than 1% of at least 1 of the 3 populations. Differential expression between M1 versus M0 and M2 versus M0 BMDMs were calculated and tested as above. Genes that showed FDR-adjusted P values less than 1×10⁻⁵ were ranked high to low according to their absolute log 2 (fold change) values, and the top 1000 of each comparison were selected. Out of the selected genes, 435 showed either both-positive or both-negative log 2 (fold change) between M1 versus M0 and M2 versus M0 BMDMs and were termed AMDSGs.

Pairwise Similarity Comparison Among M0, M1, and M2 BMDM Samples

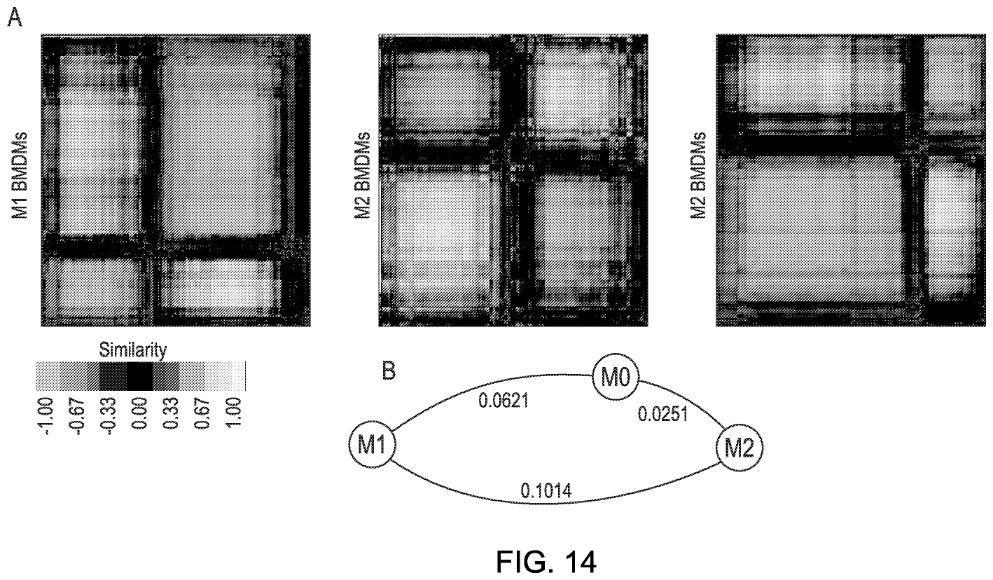

Pairwise similarities between BMDM cells were calculated using Pearson's correlation method. Pearson's correlation coefficients of each M1 versus M2 cell, M2 versus M0 cell, and M1 versus M2 cell pairs were calculated using the 11,566 genes that were expressed at greater than 1% frequency in at least 1 population of the 3, as follows:

$$r = \frac{{\sum }_{i = 1}^{n} ( {M}_{x} - \overline{M} ) ( {M}_{y} - \overline{M} )}{\sqrt{{\sum }_{i = 1}^{n} {( {M}_{X} - \overline{M} )}^{2}} \sqrt{{\sum }_{i = 1}^{n} {( {M}_{y} - \overline{M} )}^{2}}}$$

Where Mₓ and M_{y} are expression levels (UMI counts) of a certain gene in the 2 cells to be compared: S_{ct}=S−S_{avg} is the average expression of that gene in all the BMDMs, n is the total number of genes (11,566 genes) used for calculation, and r is the Pearson correlation coefficient of the 2 cells, as an indicator of similarity. A larger r means more similar. The calculated similarities were hierarchically clustered using Cluster 3.0 and visualized using Java TreeView 3.0 (jtreeview.sourceforge.net) (FIG. 14A).

Macrophage Single-Cell Transcriptome Profile Similarity Comparison

Three sets of reference transcriptome profiles were generated as the average UMI of each detected gene (UMI>1, detection frequency >1% in either M0, M1, or M2 samples) in 4736 cells of M0, M1, or M2 samples as described above, and termed the M0-average reference transcriptome (M0_{aveR}), M1_{aveR}, or M2_{aveR}.

Similarities between macrophage transcriptomes (a single-cell or bulk macrophage transcriptome profile) and reference, M0_{aveR}, M1_{aveR}, or M2_{aveR}, were calculated using a method modified from Pearson's correlation (rₘⱼ, equation listed below) in R. The PSGs and AMDSGs were first adapted to the macrophage transcriptomes, resulting in (sub)sets of PSG (PSG_{sub}) and AMDSG (AMDSG_{sub}) that were expressed in the macrophages to be tested. To emphasize the changes in gene expression during macrophage activation under various conditions, we performed a gene set centering adjustment to focus on fold-change difference of each individual gene in all samples within a study. The average expression level of a given gene (S_{avg}) was calculated as the mean of this gene in each dataset (S) in the whole study. S_{ct} is calculated as the centered value of this gene: let S be the original expression level, then the centered expression S_{ct} will be:

S_{ct}=S−S_{avg}

Accordingly, three sets of reference transcriptomes, M0_{aveR}, M1_{aveR}, and M2_{aveR}, were adjusted by centering each gene expression level as described, and termed M_{j-ctR} (j=0, 1, 2). The similarity between a macrophage transcriptome profile and one centered reference transcriptome was calculated as the following: i represents a gene ID among the PSG set (for rₘ₁ or rₘ₂ calculation) or AMDSG set (for rₘ₀ calculation); n=number of genes in PSG_{sub} (for rₘ₁, rₘ₂) or AMDSG_{sub} (for rₘ₀); j=0, 1, 2.

$${r}_{mj} = \frac{{\sum }_{i = 1}^{n} {S}_{ct i} \times {M}_{j - ctR i}}{\sqrt{{\sum }_{i = 1}^{n} {S}_{ct i}^{2}} \sqrt{{\sum }_{i = 1}^{n} {M}_{j - ctR i}^{2}}}$$

Adjusted correlation values for each comparison is provided as rₘ₀, rₘ₁, or rₘ₂ for each transcriptome profile. Each cell in an scRNA-seq data set or an RNA-seq profile in a given experiment was treated as one transcriptome profile and calculated for all 3 r values.

Similarities between the 3 sets of reference transcriptomes, M0_{aveR}, M1_{aveR}, or M2_{aveR}, were calculated following the same method as above. Adjusted correlations (r_{M1M0}, r_{M2M0}, r_{M1M2}) between the 3 populations were calculated using all the 11,566 genes that were expressed (UMI>1) in more than 1% of at least 1 of the 3 populations. The distances between the 3 populations were calculated as 1−r_{M1M0}, 1−r_{M2M0}, and 1−r_{M1M2}, respectively (FIG. 14B). Larger distances indicate more different from the other.

Generation of MPI and AMDI for Each Cell

Calculations described below were applied to all tested transcriptome profiles, including scRNA-seq and RNA-seq profiles. Transcriptome profiles generated using other platforms, such as qRT-PCR and microarray, were also tested (data not shown).

The rₘ₁ and rₘ₂ values were calculated as mentioned above for each cell in M1 or M2 BMDMs with PSG and plotted (FIG. 14C). Linear regression of the cell distribution (M1 and M2 BMDMs) on the rₘ₁−rₘ₂ plot was performed using R (FIGS. 14B and 14C). The adjusted R² value was 0.998 and the regression line was defined as the polarization axis. Let the equation of the regression line be: ax+by +c=0.

The coordinates of each sample's P were calculated as follows:

$${x}_{p} = \frac{{b}^{2} \times {r}_{m 1} - a b \times {r}_{m 2} - a c}{{a}^{2} + {b}^{2}}$$

$${y}_{p} = \frac{{a}^{2} \times {r}_{m 2} - a b \times {r}_{m 1} - b c}{{α}^{2} + {b}^{2}}$$

The most upper-left P (xₚ₀, yₚ₀) in the BMDM M1-M2 samples was set as the reference 0 point (P₀). Accordingly, the most lower-right P (xₚₘₐₓ, yₚₘₐₓ) was set as the reference Pₘₐₓ point. The distance l from the reference 0 point to a given sample's P (xₚ, yₚ) was calculated as follows:

l=\sqrt{(xₚ−xₚ₀)²+(yₚ−yₚ₀)²}

The distance between Pₘₐₓ and P₀ was scaled to a −50 (P₀) to 50 (Pₘₐₓ) range and each rescaled individual sample was termed its MPI.

Three sets of BMDM (M0, M1, and M2) scRNA-seq data were applied to compute AMDI. The rₘ₀ of each cell in BMDM data sets (M0, M1, and M2) was calculated as mentioned above using the AMDSG set. The highest and lowest rₘ₀ of the cells in the BMDM data sets were set as −50 and 50, and all the other rₘ₀ values were rescaled accordingly with linear relation, and were termed AMDI. Plots were generated using ggplot2 or our own R codes.

Heatmap Generation of Gene Expression Along the Polarization Axis

The polarization axes were divided into intervals with a given bin size and cells within a certain bin were treated as a small group. A gene expression value (log 2 transformed) was calculated as the average level (UMI, FPKM, TPM, etc.) of all cells in each bin group. In figures presented in this study, the bin size of MPI values was 5 (FIG. 13F) or 2.5 (FIG. 16D) units. The range of gene expression levels in all bins was rescaled to a 0 to 100 artificial unit range and the expression levels were accordingly transformed along this scale with linear relation. Transformed expression levels of genes in each bin group were then hierarchically clustered by the hclust function of R using the complete-link agglomeration method; distances used for clustering were calculated by the dist function using the Euclidean method. The clustered relative expression values along the polarization axis were visualized using R plotly package (plot.ly).

Cell-Trajectory Analysis

Single-cell trajectories of BMDMs were built using the Monocle package (version 2.8.0). Whole-transcriptome trajectories of BMDMs were built using the total 11,566 genes that were expressed (UMI>1) in more than 1% of at least 1 of the 3 populations. In our modified trajectory analyses, BMDMs were sorted using the 500 PSGs plus 435 AMDSGs. Dimensionality reduction was conducted using the DDRTree method and the minimum spanning tree was plotted using the plot_cell_trajectory function.

Expression levels of the signature genes along pseudotime in the 2 lineages (i.e., from M0-dominant state to M1-dominant state and from M0-dominant state to M2-dominant state) were visualized using the plot_genes_branched_heatmap function; on the generated heatmap, gene expression levels were smoothened using the VGAM package and rescaled to a −3 to 3 range and were hierarchically clustered.

Dimensionality Reduction and Clustering

t-SNE dimensionality reduction of BMDMs and ATMs based on whole transcriptomes was generated by 10× Genomics Cell Ranger pipeline (version 2.1). Dimensionality of gene-barcode matrices was first reduced to 10 principal components using principal components analysis (PCA). PCA-reduced data were further reduced to 2-dimensional space using the t-SNE method and visualized in the Loupe Cell Browser (10× Genomics) and/or by R. Graph-based clustering of cells was conducted in the PCA space; a sparse nearest-neighbor graph of the cells was built first and Louvain modularity optimization was then applied. The number of nearest neighbors was logarithmically in accordance with the number of cells. In the last step, repeated cycles of hierarchical clustering and merging of cluster pairs that had no significant differential expression was performed, until no more cluster pairs could merge.

Dimensionality reduction of ATMs and atherosclerosis macrophages based on specific PSGs and AMDSGs were conducted using the Rtsne function of R. The cell-gene matrices including only the selected signature genes were first reduced to 50 principal components by PCA, which were passed to the Barnes-Hut t-SNE algorithm to further reduce the data to 2-dimensional space. The t-SNE plots overlaid with gene expression were visualized by R.

Other Bioinformatic Analyses

Signaling pathway enrichment and upstream regulator prediction were analyzed using Ingenuity Pathway Analysis (IPA) (Qiagen), unless indicated specifically. Gene ontology analysis for each data set was performed using various platforms or online servers, including PANTHER classification system (pantherdb.org), ToppGene Suite (toppgene.cchmc.org), and NIH DAVID Bioinformatics Resources (david-d.ncifcrf.gov).

Statistics

Unless otherwise stated, P values of gene differential expression were determined by 2-tailed Welch's t test. P values of enrichment of pathways, upstream regulators, and gene ontology terms were generated by the corresponding bioinformatics tools.

Study Approval

All animal procedures were approved by and carried out in accordance with the policies of the Institutional Animal Care and Use Committee of UConn Health. Patient data were collected by P. Holvoet and complied with the Declaration of Helsinki and the Medical Ethics Committee of the Katholieke Universiteit Leuven approved the study protocol. As stated in the previous study, all human participants gave written informed consent prior to inclusion in the study.

Data Availability.

Expression profiles of human circulating CD14⁺ monocytes from obese and nonobese human subjects with and without bariatric surgery or with and without diabetes are available through NCBI GEO accession numbers GSE32575 and GSE54350, respectively. BMDM and ATM scRNA-seq data are available through NCBI GEO accession number GSE117176.

Referring to FIG. 28, a computer system for predicting risk of a patient in accordance with one or more implementations of the present disclosure is shown. It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a special purpose computer or other programmable data processing instrument to produce a machine, such that the processor-executable instructions which execute on the computer or other programmable data processing instrument create a device for implementing the functions specified in the flowchart block or blocks.

These processor-executable instructions may also be stored in a computer-readable memory or a computer-readable medium that may direct a computer or other programmable data processing instrument to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing instrument to cause a series of operational steps to be performed on the computer or other programmable instrument to produce a computer-implemented process such that the processor-executable instructions that execute on the computer or other programmable instrument provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

The method steps recited throughout this disclosure may be combined, omitted, rearranged, or otherwise reorganized with any of the figures presented herein and are not intend to be limited to the four corners of each sheet presented.

The techniques disclosed herein may be implemented on a computing device in a way that improves the efficiency of its operation. As an example, the methods, instructions, and steps disclosed herein may improve the functioning of a computing device.

The computer system 2800 may be used to predict risk of a patient. For example, the risk may related to the cardiovascular system, the endocrine system, obesity, diabetes, other morbidities, and combinations thereof. The computer system 2800 may include one or more processors 2802. The one or more processors 2802 may include or be graphics processing units, field-programmable gate arrays, application-specific integrated circuits, other implements, and combinations thereof. The one or more processors may be associated or integrated with memory 2804. The memory 2804 may be non-transitory. The memory may include a computer-readable medium or media. The computer system may include interfaces 2806 for communicating with peripheral components and one or more displays 2808. For example, the interfaces 2806 may communicate with a data acquisition unit 2810 (DAQ), repository, or another implement to obtain sequencing data of monocytes obtained from a patient. The sequencing data of monocytes may be performed in-office or elsewhere. The computer system 2800 may be configured to treat the patient based on the sequencing data.

The one or more processors 2802 may be configured to characterize genetic information obtained from the sequencing data. The characterization may be based upon an expression pattern associated with inflammatory foaming macrophage activity. The one or more processors 2802 may be configured to apply a logistic regression model to the characterized genetic information to estimate a probability of a cardiovascular event by the patient. Treatment may be provided based on the probability. The logistic regression model may be stored in the memory 2804 or another repository. The logistic regression model may be based on various methods and steps described herein. The logistic regression model may include model coefficients determined based on expression patterns and outcome reports of patients for which cardiovascular events previously occurred.

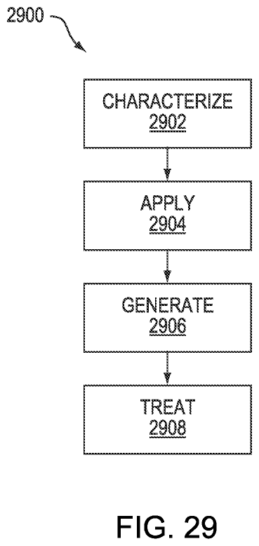

Referring to FIG. 29, a method 2900 for predicting risk of a patient in accordance with one or more implementations of the present disclosure. The computer system 2800 may be used to predict risk of a patient. For example, the risk may related to the cardiovascular system, the endocrine system, obesity, diabetes, other morbidities, and combinations thereof. In step 2902, genetic information may be characterized. The genetic information may be obtained from sequencing data. The sequencing data may be of monocytes. The monocytes may be obtained from a patient. In step 2904, a logistic regression model may be applied to the characterized genetic information. For example, the application of the logistic regression model may estimate the probability or risk of an event. The risk may related to the cardiovascular system, the endocrine system, obesity, diabetes, other morbidities, and combinations thereof. The genetic information may be characterized according to an expression pattern associated with inflammatory macrophage activity. The genetic information may be characterized according to a comparison of the genetic information to a candidate gene set. The candidate gene set may include functionally interrelated genes. A gene of the candidate gene set may be associated with an inflammation or immune response. The gene of the candidate gene set may be associated with a lipid transport or metabolism. The gene of the candidate gene set may be associated with cell proliferation or development. The gene of the candidate gene set may be associated with one or more of inflammation or immune response, lipid transport or metabolism, or cell proliferation or development.

The logistic regression model may be trained or based on physiological feature data and demographic data associated with the subject. Physiological feature data and demographic data may be applied as an input to the logistic regression model. The logistic regression model may be trained or based on a generated macrophage polarization index (MPI) score. The generated MPI score may be applied as an input to the logistic regression model.

A macrophage-derived foam cell index (MDFI) score may be generated. The Genetic information may be characterized according to MDFI. The logistic regression model may be trained or based on the MDFI, and application of the logistic regression model may include e inputting the generated MDFI score to the model.

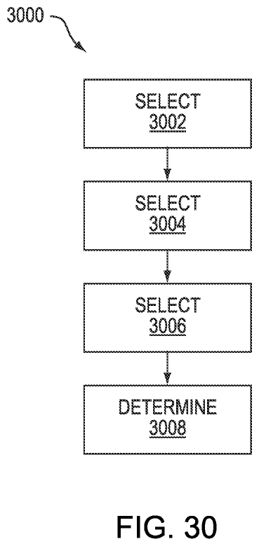

Referring to FIG. 30, shows a method 3000 for training a machine learning algorithm in accordance with one or more implementations of the present disclosure. Although four steps are shown, the method may include more steps as described herein. In step 3002, the machine learning algorithm may be trained to include 20 batches determined as follows. For a batch, 5,000 gene sets may be selected from the 2209-gene pool. The gene sets may contain 25 to 30 genes. Multivariable logistic regression modeling may be conducted using each gene set plus MPI and selected MESA features, and the gene set that produced the highest AUC in model testing was selected as the candidate gene set of the batch. From this step, 15 of the 20 batch candidate gene sets that had the highest AUCs were selected and pooled, which may be used as the gene pool in step 3004.

In step 3004, for a batch, 5,000 gene sets may be selected from the 15-gene pool. The gene sets may contain 25 to 30 genes. Multivariable logistic regression modeling may be conducted using each gene set plus MPI and selected MESA features, and the gene set that produced the highest AUC in model testing was selected as the candidate gene set of the batch. From this step, 15 of the 20 batch candidate gene sets that had the highest AUCs were selected and pooled, which may be used as the gene pool in step 3006.

In step 3006, for a batch, 5,000 gene sets may be selected from the 15-gene pool. The gene sets may contain 25 to 30 genes. Multivariable logistic regression modeling may be conducted using each gene set plus MPI and selected MESA features, and the gene set that produced the highest AUC in model testing was selected as the candidate gene set of the batch. From this step, 12 of the 15 batch candidate gene sets that had the highest AUCs were selected and pooled, which may be used as the gene pool in subsequent steps. The following steps in the training process may include the top 8, 5, 3, 2, and 1 candidate gene sets that had the highest AUCs may be used as the gene pool for the following step. The training process may include seven such steps. In step 3008, the final CVD residual risk signature genes may be determined.

1. A method of predicting metabolic risk of a patient, comprising:
characterizing, based on an expression pattern associated with inflammatory foaming macrophage activity of the patient, genetic information obtained from sequencing data of monocytes of the patient;
determining, based on an application of a predictive model to the characterized genetic information, a probability of a metabolic event by the patient; and
causing, based on the sequencing data and the probability of the metabolic event by the patient, a treatment of the patient.

2. The method of claim 1, wherein the predictive model comprises a logistic regression machine learning model, wherein the logistic regression machine learning model is trained based on physiological feature data and demographic data associated with a plurality of patients.

3. The method of claim 1, wherein characterizing the genetic information comprises comparing the genetic information to a candidate gene set.

4. The method of claim 3, wherein the candidate gene set comprises functionally interrelated genes, wherein each gene of the candidate gene set is associated with at least one function comprising an inflammation or immune response, lipid transport or metabolism, or cell proliferation or development.

5. The method of claim 1, wherein the metabolic event is associated with one or more of cardiovascular disease, obesity, or diabetes.

6. The method of claim 1, wherein characterizing the genetic information comprises generating a macrophage polarization index (MPI) score, wherein applying the predictive model to the characterized genetic information to estimate the probability of the metabolic event comprises inputting the generated MPI score to the predictive model to estimate the probability of the metabolic event.

7. The method of claim 1, wherein characterizing the genetic information comprises generating a macrophage-derived foam cell index (MDFI) score, and wherein applying the predictive model to the characterized genetic information to estimate the probability of the metabolic event comprises inputting the generated MDFI score to the predictive model to estimate the probability of the metabolic event.

8. (canceled)

9. (canceled)

10. A computer system for predicting a metabolic risk of a patient, comprising:
a data source configured to provide sequencing data of monocytes obtained from the patient; and
a processor in communication with the data source, wherein the processor is configured to:
characterize genetic information obtained from the sequencing data based an expression pattern associated with inflammatory foaming macrophage activity of the patient;
determine a probability of a metabolic event by the patient based on an application of a predictive model to the characterized genetic information; and
cause a treatment of the patient based on the sequencing data and the probability of the metabolic event by the patient.

11. The computer system of claim 10, wherein the predictive model comprises a logistic regression machine learning model, wherein the logistic regression machine learning model is trained based on physiological feature data and demographic data associated with a plurality of patients.

12. (canceled)

13. (canceled)

14. The computer system of claim 10, wherein the processor is configured to characterize the genetic information by comparing the genetic information to a candidate gene set.

15. The computer system of claim 14, wherein the candidate gene set comprises functionally interrelated genes, wherein each gene of the candidate gene set is associated with at least one function comprising an inflammation or immune response, lipid transport or metabolism, or cell proliferation or development.

16. The computer system of claim 10, wherein the metabolic event is associated with one or more of cardiovascular disease, obesity, or diabetes.

17. The computer system of claim 10, wherein the processor is configured to characterize the genetic information by generating a macrophage polarization index (MPI) score, wherein the processor is configure to apply the predictive model to the characterized genetic information to estimate the probability of the metabolic event, the processor is further configured to input the generated MPI score to the predictive model to estimate the probability of the metabolic event.

18. The computer system of claim 10, wherein the processor is configured to characterize the genetic information by generating a macrophage-derived foam cell index (MDFI) score, and wherein the processor is configured to apply the predictive model to the characterized genetic information to estimate the probability of the metabolic event, the processor is further configured to input the generated MDFI score to the predictive model to estimate the probability of the metabolic event.

19. (canceled)

20. An apparatus for predicting a metabolic risk of a patient, comprising:
one or more processors; and
a memory storing processor-executable instructions that, when executed by the one or more processors, cause the apparatus to:
characterize, based on an expression pattern associated with inflammatory foaming macrophage activity of the patient, genetic information obtained from sequencing data of monocytes of the patient;
determine, based on an application of a predictive model to the characterized genetic information, a probability of a metabolic event by the patient; and
cause, based on the sequencing data and the probability of the metabolic event by the patient, a treatment of the patient.

21. The apparatus of claim 20, wherein the predictive model comprises a logistic regression machine learning model, wherein the logistic regression machine learning model is trained based on physiological feature data and demographic data associated with a plurality of patients.

22. The apparatus of claim 20, wherein the processor-executable instructions that, when executed by the one or more processors, cause the apparatus to characterize the genetic information, further cause the apparatus to compare the genetic information to a candidate gene set.

23. The apparatus of claim 20, wherein the metabolic event is associated with one or more of cardiovascular disease, obesity, or diabetes.

24. The apparatus of claim 20, wherein the processor-executable instructions that, when executed by the one or more processors, cause the apparatus to characterize the genetic information, further cause the apparatus to generate a macrophage polarization index (MPI) score, and wherein the processor-executable instructions that, when executed by the one or more processors, cause the apparatus to apply the predictive model to the characterized genetic information to estimate the probability of the metabolic event, further cause the apparatus to input the generated MPI score to the predictive model to estimate the probability of the metabolic event.

25. The apparatus of claim 20, wherein the processor-executable instructions that, when executed by the one or more processors, cause the apparatus to characterize the genetic information, further cause the apparatus to generate a macrophage-derived foam cell index (MDFI) score, and wherein the processor-executable instructions that, when executed by the one or more processors, cause the apparatus to apply the predictive model to the characterized genetic information to estimate the probability of the metabolic event, further cause the apparatus to input the generated MDFI score to the predictive model to estimate the probability of the metabolic event.
